SEQUENCE LISTING (105 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 3 - protein, 19 aa, Artificial Sequence
  source: /note=“Description of Artificial Sequence: Synthetic peptide”
DGNEEMGGITQTPYKVSIS

SEQ ID NO: 4 - protein, 19 aa, Artificial Sequence
  source: /note=“Description of Artificial Sequence: Synthetic peptide”
DGNEEMGSITQTPYQVSIS

SEQ ID NO: 5 - protein, 256 aa, Artificial Sequence
  source: /note=“Description of Artificial Sequence: Synthetic polypeptide”
QDGNEEMGGITQTPYKVSISGTTVILTCDIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 256 aa, Artificial Sequence
  source: /note=“Description of Artificial Sequence: Synthetic polypeptide”
QDGNEEMGSITQTPYQVSISGTTVILTCDIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 261 aa, Artificial Sequence
  source: /note=“Description of Artificial Sequence: Synthetic polypeptide”
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVDIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 8 - protein, 261 aa, Artificial Sequence; identical to SEQ ID NO: 7
  source: /note=“Description of Artificial Sequence: Synthetic polypeptide”

SEQ ID NO: 9 - protein, 352 aa, Artificial Sequence
  source: /note=“Description of Artificial Sequence: Synthetic polypeptide”
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGGGGSGGGGSGGGGSASPAAFKIPIEELEDRVFVNCNTSITWVEGTVGTLLSDITRLDLGKRILDPRGIYRCNGTDIYKDKESTVQVHYRMDIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 10 - protein, 347 aa, Artificial Sequence
  source: /note=“Description of Artificial Sequence: Synthetic polypeptide”
QDGNEEMGSITQTPYQVSISGTTVILTCSQHLGSEAQWQHNGKNKEDSGDRLFLPEFSEMEQSGYYVCYPRGSNPEDASHHLYLKARVIDYKGGGGSGGGGSGGGGSASPAAFKIPVEELEDRVFVKCNTSVTWVEGTVGTLLTNNTRLDLGKRILDPRGIYRCNGTDIYKDKESAVQVHYRMDIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 11 - protein, 347 aa, Artificial Sequence; identical to SEQ ID NO: 10
  source: /note=“Description of Artificial Sequence: Synthetic polypeptide”

SEQ ID NO: 12 - skipped

SEQ ID NO: 13 - protein, 123 aa, Artificial Sequence
  source: /note=“Description of Artificial Sequence: Synthetic polypeptide”
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGYSAEFAHRSGLDVWGQGTLVTVSS

SEQ ID NO: 14 - protein, 123 aa, Artificial Sequence
  source: /note=“Description of Artificial Sequence: Synthetic polypeptide”
EVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 15 - protein, 123 aa, Artificial Sequence
  source: /note=“Description of Artificial Sequence: Synthetic polypeptide”
EVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 16 - protein, 112 aa, Artificial Sequence
  source: /note=“Description of Artificial Sequence: Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWDHHRSGAVFGGGTKLTVLGQ

SEQ ID NO: 17 - protein, 112 aa, Artificial Sequence
  source: /note=“Description of Artificial Sequence: Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 18 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNIRPSGVPDRFSGSKSGTSASLAISGLRSEDEADYYCAAWDRHSHGAVFGGGTKLTVLGQ

SEQ ID NO: 19 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNKRPSGVPDRFSGSKSGTSASLAISGLRSEDEADYYCAAWDRHSHGAVFGGGTKLTVLGQ

SEQ ID NO: 20 - protein, 112 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNKKRPSGVPDRFSGSKSGTSASLAISGLRSEDEADYYCAAWDRHSHGAVFGGGTKLTVLGQ

SEQ ID NO: 21 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNKQRPSGVPDRFSGSKSGTSASLAISGLRSEDEADYYCAAWDRHSHGAVFGGGTKLTVLGQ

SEQ ID NO: 22 - protein, 112 aa, Artificial Sequence
  source: /note=“Description of Artificial Sequence: Synthetic polypeptide”
QSVLTQPPSASGTPGQRVTISCSGSSSNIGQTYVYWYQQLPGTAPKLLIYRNSQRPSGVPDRFSGSKSGTSASLAISGLRSEDEADYYCAAWDRHSHGAVFGGGTKLTVLGQ

SEQ ID NO: 23 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNHIRPSGVPDRFSGSKSGTSASLAISGLRSEDEADYYCAAWDRHSHGAVFGGGTKLTVLGQ

SEQ ID NO: 24 - protein, 112 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 25 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNQRPSGVPDRFSGSKSGTSASLAISGLRSEDEADYYCAAWDRHSHGAVFGGGTKLTVLGQ

SEQ ID NO: 26 - protein, 112 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNKQRPSGVPDRFSGSKSGTSASLAISGLRSEDEADYYCAAWDRHSHGAVFGGGTKLTVLGQ

SEQ ID NO: 27 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNTQRPSGVPDRFSGSKSGTSASLAISGLRSEDEADYYCAAWDRHSHGAVFGGGTKLTVLGQ

SEQ ID NO: 28 - protein, 112 aa, Artificial Sequence
  source: /note=“Description of Artificial Sequence: Synthetic polypeptide”
QSVLTQPPSASGTPGQRVTISCSGSSSNIGKNYVYWYQQLPGTAPKLLIYRNYQRPSGVPDRFSGSKSGTSASLAISGLRSEDEADYYCAAWDRHSHGAVFGGGTKLTVLGQ

SEQ ID NO: 29 - protein, 112 aa, Artificial Sequence
  source: /note=“Description of Artificial Sequence: Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAGWDRRLHGAVFGGGTKLTVLGQ

SEQ ID NO: 30 - protein, 112 aa, Artificial Sequence
  source: /note=“Description of Artificial Sequence: Synthetic polypeptide”
QSVLTQPPSASGTPGQRVTISCSGSSSNIGQTYVYWYQQLPGTAPKLLIYRNTQRPSGVPDRFSGSKSGTSASLAISGLRSEDEADYYCAAWDRHSHGAVFGGGTKLTVLGQ

SEQ ID NO: 31 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNKRPSGVPDRFSGSKSGTSASLAISGLRSEDEADYYCAGWDRRLHGAVFGGGTKLTVLGQ

SEQ ID NO: 32 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNHIRPSGVPDRFSGSKSGTSASLAISGLRSEDEADYYCAGWDRRLHGAVFGGGTKLTVLGQ

SEQ ID NO: 33 - protein, 112 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNNQRPSGVPDRFSGSKSGTSASLAISGLRSEDEADYYCAGWDRRLHGAVFGGGTKLTVLGQ

SEQ ID NO: 34 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNQRPSGVPDRFSGSKSGTSASLAISGLRSEDEADYYCAGWDRRLHGAVFGGGTKLTVLGQ

SEQ ID NO: 35 - protein, 112 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNKQRPSGVPDRFSGSKSGTSASLAISGLRSEDEADYYCAGWDRRLHGAVFGGGTKLTVLGQ

SEQ ID NO: 36 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNTQRPSGVPDRFSGSKSGTSASLAISGLRSEDEADYYCAGWDRRLHGAVFGGGTKLTVLGQ

SEQ ID NO: 37 - protein, 112 aa, Artificial Sequence
  source: /note=“Description of Artificial Sequence: Synthetic polypeptide”
QSVLTQPPSASGTPGQRVTISCSGSSSNIGQTYVYWYQQLPGTAPKLLIYRNTQRPSGVPDRFSGSKSGTSASLAISGLRSEDEADYYCAGWDRRLHGAVFGGGTKLTVLGQ

SEQ ID NO: 38 - protein, 112 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNIRPSGVPDRFSGSKSGTSASLAISGLRSEDEADYYCAGWDRRLHGAVFGGGTKLTVLGQ

SEQ ID NO: 39 - protein, 14 aa, Artificial Sequence
  source: /note=“Description of Artificial Sequence: Synthetic peptide”
GYSAEFAHRSGLDV

SEQ ID NO: 40 - protein, 10 aa, Artificial Sequence
  source: /note=“Description of Artificial Sequence: Synthetic peptide”
GFTFSSYWMS

SEQ ID NO: 41 - protein, 10 aa, Artificial Sequence
  source: /note=“Description of Artificial Sequence: Synthetic peptide”
GFTFKSYYMS

SEQ ID NO: 42 - protein, 10 aa, Artificial Sequence
  source: /note=“Description of Artificial Sequence: Synthetic peptide”
GFTFRSHYMT

SEQ ID NO: 43 - protein, 17 aa, Artificial Sequence
  source: /note=“Description of Artificial Sequence: Synthetic peptide”
NIKQDGSEKYYVDSVKG

SEQ ID NO: 44 - protein, 17 aa, Artificial Sequence
  source: /note=“Description of Artificial Sequence: Synthetic peptide”
NIDYQSQHAYYAESVKG

SEQ ID NO: 45 - protein, 17 aa, Artificial Sequence
  source: /note=“Description of Artificial Sequence: Synthetic peptide”
NIDYEGTRTYYAESVKG

SEQ ID NO: 46 - protein, 13 aa, Artificial Sequence
  source: /note=“Description of Artificial Sequence: Synthetic peptide”
SGSSSNIGSNYVY

SEQ ID NO: 47 - protein, 13 aa, Artificial Sequence
  source: /note=“Description of Artificial Sequence: Synthetic peptide”
SGSSSNIGINYVY

SEQ ID NO: 48 - protein, 13 aa, Artificial Sequence
  source: /note=“Description of Artificial Sequence: Synthetic peptide”
SGSSSNIGANYVY

SEQ ID NO: 49 - protein, 13 aa, Artificial Sequence
  source: /note=“Description of Artificial Sequence: Synthetic peptide”
SGSSSNIGQTYVY

SEQ ID NO: 50 - protein, 13 aa, Artificial Sequence
  source: /note=“Description of Artificial Sequence: Synthetic peptide”
SGSSSNIGKNYVY

SEQ ID NO: 51 - protein, 7 aa, Artificial Sequence
  source: /note=“Description of Artificial Sequence: Synthetic peptide”
RNNQRPS

SEQ ID NO: 52 - protein, 7 aa, Artificial Sequence
  source: /note=“Description of Artificial Sequence: Synthetic peptide”
RNNIRPS

SEQ ID NO: 53 - protein, 7 aa, Artificial Sequence
  source: /note=“Description of Artificial Sequence: Synthetic peptide”
RNNKRPS

SEQ ID NO: 54 - protein, 7 aa, Artificial Sequence
  source: /note=“Description of Artificial Sequence: Synthetic peptide”
RNKKRPS

SEQ ID NO: 55 - protein, 7 aa, Artificial Sequence
  source: /note=“Description of Artificial Sequence: Synthetic peptide”
RNKQRPS

SEQ ID NO: 56 - protein, 7 aa, Artificial Sequence
  source: /note=“Description of Artificial Sequence: Synthetic peptide”
RNSQRPS

SEQ ID NO: 57 - protein, 7 aa, Artificial Sequence
  source: /note=“Description of Artificial Sequence: Synthetic peptide”
RNHIRPS

SEQ ID NO: 58 - protein, 7 aa, Artificial Sequence
  source: /note=“Description of Artificial Sequence: Synthetic peptide”
RNTQRPS

SEQ ID NO: 59 - protein, 7 aa, Artificial Sequence
  source: /note=“Description of Artificial Sequence: Synthetic peptide”
RNYQRPS

SEQ ID NO: 60 - protein, 11 aa, Artificial Sequence
  source: /note=“Description of Artificial Sequence: Synthetic peptide”
AAWDHHRSGAV

SEQ ID NO: 61 - protein, 11 aa, Artificial Sequence
  source: /note=“Description of Artificial Sequence: Synthetic peptide”
AAWDRHSHGAV

SEQ ID NO: 62 - protein, 11 aa, Artificial Sequence
  source: /note=“Description of Artificial Sequence: Synthetic peptide”
AGWDRRLHGAV

SEQ ID NO: 63 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGSNYVYWYQQLPGTAPKLLIYRNNQRPSGVPDRFSGSKSGTSASLAISGLRSEDEADYYCAAWDHHRSGAVFGGGTKLTVLGQ

SEQ ID NO: 64 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQ

SEQ ID NO: 65 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNIRPSGVPDRFSGSKSGTSASLAISGLRSEDEADYYCAAWDRHSHGAVFGGGTKLTVLGQ

SEQ ID NO: 66 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNKRPSGVPDRFSGSKSGTSASLAISGLRSEDEADYYCAAWDRHSHGAVFGGGTKLTVLGQ

SEQ ID NO: 67 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNKKRPSGVPDRFSGSKSGTSASLAISGLRSEDEADYYCAAWDRHSHGAVFGGGTKLTVLGQ

SEQ ID NO: 68 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNKQRPSGVPDRFSGSKSGTSASLAISGLRSEDEADYYCAAWDRHSHGAVFGGGTKLTVLGQ

SEQ ID NO: 69 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGQTYVYWYQQLPGTAPKLLIYRNSQRPSGVPDRFSGSKSGTSASLAISGLRSEDEADYYCAAWDRHSHGAVFGGGTKLTVLGQ

SEQ ID NO: 70 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNHIRPSGVPDRFSGSKSGTSASLAISGLRSEDEADYYCAAWDRHSHGAVFGGGTKLTVLGQ

SEQ ID NO: 71 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQ

SEQ ID NO: 72 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNQRPSGVPDRFSGSKSGTSASLAISGLRSEDEADYYCAAWDRHSHGAVFGGGTKLTVLGQ

SEQ ID NO: 73 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNKQRPSGVPDRFSGSKSGTSASLAISGLRSEDEADYYCAAWDRHSHGAVFGGGTKLTVLGQ

SEQ ID NO: 74 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNTQRPSGVPDRFSGSKSGTSASLAISGLRSEDEADYYCAAWDRHSHGAVFGGGTKLTVLGQ

SEQ ID NO: 75 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGKNYVYWYQQLPGTAPKLLIYRNYQRPSGVPDRFSGSKSGTSASLAISGLRSEDEADYYCAAWDRHSHGAVFGGGTKLTVLGQ

SEQ ID NO: 76 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGSNYVYWYQQLPGTAPKLLIYRNNQRPSGVPDRFSGSKSGTSASLAISGLRSEDEADYYCAGWDRRLHGAVFGGGTKLTVLGQ

SEQ ID NO: 77 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGQTYVYWYQQLPGTAPKLLIYRNTQRPSGVPDRFSGSKSGTSASLAISGLRSEDEADYYCAAWDRHSHGAVFGGGTKLTVLGQ

SEQ ID NO: 78 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNKRPSGVPDRFSGSKSGTSASLAISGLRSEDEADYYCAGWDRRLHGAVFGGGTKLTVLGQ

SEQ ID NO: 79 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNHIRPSGVPDRFSGSKSGTSASLAISGLRSEDEADYYCAGWDRRLHGAVFGGGTKLTVLGQ

SEQ ID NO: 80 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNNQRPSGVPDRFSGSKSGTSASLAISGLRSEDEADYYCAGWDRRLHGAVFGGGTKLTVLGQ

SEQ ID NO: 81 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNQRPSGVPDRFSGSKSGTSASLAISGLRSEDEADYYCAGWDRRLHGAVFGGGTKLTVLGQ

SEQ ID NO: 82 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGANYVYWYQQLPGTAPKLLIYRNKQRPSGVPDRFSGSKSGTSASLAISGLRSEDEADYYCAGWDRRLHGAVFGGGTKLTVLGQ

SEQ ID NO: 83 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNTQRPSGVPDRFSGSKSGTSASLAISGLRSEDEADYYCAGWDRRLHGAVFGGGTKLTVLGQ

SEQ ID NO: 84 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGQTYVYWYQQLPGTAPKLLIYRNTQRPSGVPDRFSGSKSGTSASLAISGLRSEDEADYYCAGWDRRLHGAVFGGGTKLTVLGQ

SEQ ID NO: 85 - protein, 340 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGQSVLTQPPSASGTPGQRVTISCSGSSSNIGINYVYWYQQLPGTAPKLLIYRNNIRPSGVPDRFSGSKSGTSASLAISGLRSEDEADYYCAGWDRRLHGAVFGGGTKLTVLGQ

SEQ ID NO: 86 - protein, 214 aa, Artificial Sequence
  source: /note=“Description of Artificial Sequence: Synthetic polypeptide”
DIQMTQSPSSLSASVGDRVTITCRASQDVNTAVAWFQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 351 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGYSAEFAHRSGLDVWGQGTLVTVSS

SEQ ID NO: 88 - protein, 351 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSS

SEQ ID NO: 89 - protein, 351 aa, Artificial Sequence
  source: /note=“Description of Artificial Sequence: Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGQPSGEVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSS

SEQ ID NO: 90 - protein, 10 aa, Artificial Sequence
  source: /note=“Description of Artificial Sequence: Synthetic peptide”
  VARIANT (5)..(5): /replace=“Lys” or “Arg”
  VARIANT (7)..(7): /replace=“His”
  VARIANT (8)..(8): /replace=“Tyr”
  VARIANT (10)..(10): /replace=“Thr”
  SITE (1)..(10): /note=“Variant residues given in the sequence have no preference with respect to those in the annotations for variant positions”
GFTFSSYWMS

SEQ ID NO: 91 - protein, 17 aa, Artificial Sequence
  source: /note=“Description of Artificial Sequence: Synthetic peptide”
  VARIANT (3)..(3): /replace=“Asp”
  VARIANT (4)..(4): /replace=“Tyr”
  VARIANT (5)..(5): /replace=“Gln” or “Glu”
  VARIANT (6)..(6): /replace=“Gly”
  VARIANT (7)..(7): /replace=“Gln” or “Thr”
  VARIANT (8)..(8): /replace=“His” or “Arg”
  VARIANT (9)..(9): /replace=“Ala” or “Thr”
  VARIANT (12)..(12): /replace=“Ala”
  VARIANT (13)..(13): /replace=“Glu”
  SITE (1)..(17): /note=“Variant residues given in the sequence have no preference with respect to those in the annotations for variant positions”
NIKQDSSEKYYVDSVKG

SEQ ID NO: 92 - protein, 13 aa, Artificial Sequence
  source: /note=“Description of Artificial Sequence: Synthetic peptide”
  VARIANT (9)..(9): /replace=“Ile” or “Ala” or “Lys” or “Gln”
  VARIANT (10)..(10): /replace=“Thr”
  SITE (1)..(13): /note=“Variant residues given in the sequence have no preference with respect to those in the annotations for variant positions”
SGSSSNIGSNYVY

SEQ ID NO: 93 - protein, 7 aa, Artificial Sequence
  source: /note=“Description of Artificial Sequence: Synthetic peptide”
  VARIANT (3)..(3): /replace=“Lys” or “Ser” or “His” or “Thr” or “Tyr”
  VARIANT (4)..(4): /replace=“Ile” or “Lys”
  SITE (1)..(7): /note=“Variant residues given in the sequence have no preference with respect to those in the annotations for variant positions”
RNNQRPS

SEQ ID NO: 94 - protein, 11 aa, Artificial Sequence
  source: /note=“Description of Artificial Sequence: Synthetic peptide”
  VARIANT (2)..(2): /replace=“Gly”
  VARIANT (5)..(5): /replace=“Arg”
  VARIANT (6)..(6): /replace=“Arg”
  VARIANT (7)..(7): /replace=“Ser” or “Leu”
  VARIANT (8)..(8): /replace=“His”
  SITE (1)..(11): /note=“Variant residues given in the sequence have no preference with respect to those in the annotations for variant positions”
AAWDHHRSGAV

SEQ ID NO: 95 - protein, 453 aa, Artificial Sequence
  source: /note=“Description of Artificial Sequence: Synthetic polypeptide”
EVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 216 aa, Artificial Sequence
  source: /note=“Description of Artificial Sequence: Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWDRHSHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - protein, 226 aa, Artificial Sequence
  source: /note=“Description of Artificial Sequence: Synthetic polypeptide”
EVQLVESGGGLVQPGGSLRLSCAASGFTFKSYYMSWVRQAPGKGLEWVANIDYQSQHAYYAESVKGRFTISRDNAKNSLYLQMNSLRAEDTAVYYCARGYSAEFAHRSGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 98 - protein, 453 aa, Artificial Sequence
  source: /note=“Description of Artificial Sequence: Synthetic polypeptide”
EVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 226 aa, Artificial Sequence
  source: /note=“Description of Artificial Sequence: Synthetic polypeptide”
EVQLVESGGGLVQPGGSLRLSCAASGFTFRSHYMTWVRQAPGKGLEWVANIDYEGTRTYYAESVKGRFTISRDNAKNSLYLQMNSLRAEDTAVYYCARGYSAEFAHRSGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 100 - protein, 216 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNIRPSGVPDRFSGSKSGTSASLAISGLRSEDEADYYCAAWDRHSHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - protein, 216 aa, Artificial Sequence
  source: /note=“Description of Artificial Sequence: Synthetic polypeptide”
QSVLTQPPSASGTPGQRVTISCSGSSSNIGANYVYWYQQLPGTAPKLLIYRNNQRPSGVPDRFSGSKSGTSASLAISGLRSEDEADYYCAAWDRHSHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 216 aa, Artificial Sequence
  source: /note=“Description of Artificial Sequence: Synthetic polypeptide”
QSVLTQPPSASGTPGQRVTISCSGSSSNIGINYVYWYQQLPGTAPKLLIYRNNQRPSGVPDRFSGSKSGTSASLAISGLRSEDEADYYCAAWDRHSHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 216 aa, Artificial Sequence
  source: /note=“Description of Artificial Sequence: Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAGWDRRLHGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 104 - protein, 252 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVVIDYKDDDDKIEGRMDKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRLVNSRGLNDIFEAQKIEWHE

SEQ ID NO: 105 - protein, 5 aa, Artificial Sequence
  source: /note=“Description of Artificial Sequence: Synthetic peptide”
GQPSG

(19) United States
(12) Patent Application Publication
(10) Pub. No.: US 2023/0357398 A1
(43) Pub. Date: Nov. 9, 2023

(54) NOVEL HUMAN ANTIBODIES BINDING TO HUMAN CD3 EPSILON

(71) Applicant: MORPHOSYS AG, Planegg (DE)

(72) Inventors: Andreas BÜLTMANN, Planegg (DE); Steffen RUNZ, Heidelberg (DE); Tschimegma BATAA, Munich (DE); Markus MOOSMEIER, Munich (DE); Nicole HAUBST, Munich (DE); Jürgen KLATTIG, Peißenberg (DE); Yvonne STARK, Munich (DE)

(21) Appl. No.: 18/246,052

(22) PCT Filed: Sep. 22, 2021
(86) PCT No.: PCT/EP2021/076052

§ 371 Date: Mar. 21, 2023

(30) Foreign Application Priority Data
Sep. 24, 2020 (EP) 20197975.4

(51) Int. Cl. C07K 16/28; A61P 35/00

(52) U.S. Cl. CPC C07K 16/2809; A61P 35/00; C07K 2317/565; C07K 2317/33; C07K 2317/622; C07K 2317/55; C07K 2317/31

(57) ABSTRACT

The present disclosure provides novel fully human antibodies and antibody fragments specific for human CD3 epsilon.

### FIELD OF THE INVENTION

The present disclosure relates to a fully human antibody as well as improved variants thereof which bind to human CD3 epsilon. The present disclosure also provides multispecific antibody formats comprising such human CD3 specific antibodies and antibody fragments. The disclosure also relates to nucleic acids, vectors and host cells capable of expressing such antibodies, pharmaceutical compositions comprising said antibodies and uses of said antibodies and pharmaceutical compositions for the treatment of specific diseases.

### BACKGROUND

CD3 is a homodimeric or heterodimeric antigen expressed on T cells in association with the T cell receptor complex (TCR) and is required for T cell activation. CD3 is formed by the dimeric association of two of four different chains: epsilon, zeta, delta and gamma. The CD3 dimeric arrangements include gamma/epsilon, delta/epsilon and zeta/zeta.

Antibodies binding to CD3 have been shown to cluster CD3 on T cells, thereby causing T cell activation in a manner similar to the engagement of the T-Cell receptor (TCR) by peptide-loaded MHC molecules. CD3 specific antibodies have been proposed for therapies involving the activation of T cells. Accordingly, bi-, or multispecific antibody formats that co-engage CD3 and one or more cancer associated antigens (CAA) have been developed to redirect T-cells to attack and lyse cancer cells. While the CD3 targeting approach has shown considerable promise, the therapeutic use of such multispecific antibody formats described in the art share some common downsides. First, the absence of specific tumor antigens. Indeed, cancer associated antigens which are usually targeted are often shared by tumors and non-malignant cells. Second, a major problem of multispecific antibody formats relying on the use of CD3 specific antibody fragments (e.g. single-chain variable fragment (scFv)) is the inherent ability of these fragments to activate T cells, irrespective of binding to the target antigen (e.g. the cancer associated antigen). Indeed, many side effects observed in context of CD3 based antibody therapy appear to be associated with errant T cell function (such as the associated production of cytokines which may lead to a toxic cytokine release syndrome).

A novel approach for a multispecific antibody format which overcomes the aforementioned shortcomings for antibody based therapeutics that co-engage CD3 and cancer associated antigens is described in WO2013/104804 [JULIUS-MAXIMILIANS-UNIVERSITAT WURZBURG] and refers to “DUAL ANTIGEN-INDUCED BIPARTITE FUNCTIONAL COMPLEMENTATION”. This approach or multispecific antibody format is referred herein as “CyCAT®” (Cytotoxic cell activation at tumour).

As part of the aforementioned approach, two polypeptides are designed, each composed of an targeting moiety (e.g. a single-chain variable fragment (scFv) or an antibody Fab fragment) fused to either the variable light chain domain (VL) or the complementary variable heavy chain (VH) domain of a T cell-activating anti-CD3 antibody. Such dissected or splitted Fv domain is referred herein as “Split Effector Domain” whereas the re-assembled Fv domain reflects the “Effector Domain”.

The unpaired VH or VL domain (e.g. the split CD3 VH domain and the split CD3 VL domain) is not able to bind to CD3 alone. However, once the aforementioned two polypeptides bind to their target antigen on the surface of a cell via their targeting moiety, the complementary VL and VH domain of the CD3 specific antibody come in close proximity and interact with each other to reconstitute the original CD3 antibody (e.g. the CD3 specific antibody Fv domain). The thus on-target cell formed trispecific heterodimeric molecule engages and stimulates T cells for cancer cell destruction such as a conventional trivalent bi- or trispecifc antibody formats.

One of the critical aspects in the aforementioned approach relies in the identification of a suitable CD3 specific antibody. Such antibody does not only has to address the requirements for a conventional bispecific antibody format in terms of species cross-reactivity, affinity, potency, safety (e.g. therapeutic window) and immunogenicity but also the specific requirements of the CyCAT format.

As described above, in the CyCAT approach the employed CD3 specific Fv domain must be splitable or dissectible. This feature implies several specific requirements for the CD3 antibody.

First, the VH and VL domain of the splitted Fv domain must be producible in the CyCAT format (e.g. as unpaired CyCAT polypeptides) with acceptable purity, acceptable yield as well as acceptable monomer content. This is in particular challenging as unpaired VH or VL domains tend to form strong aggregates after production. This aggregation tendency is i.a. dependent on the particular antibody sequence and appears to be more critical for polypeptides carrying the unpaired VH domain. Indeed, the inventors of the presently claimed new CD3 specific antibodies discovered that even only slight variations in the CDR sequences of antibody variants derived from one parental antibody reveal strong differences in producibility in the CyCAT format.

Secondly, the CD3 specific antibody (e.g. the Fv domain) must retain its functional activity in terms of activating T cells and/or redirecting T cell killing to cancer cells once the unpaired CD3 specific VH and VL domain present in the set of CyCAT polypeptides are paired.

Thirdly, the unpaired CD3 specific VH or VL domain or the respective unpaired CyCAT polypeptides comprising the same should not bind to CD3 alone.

Fourthly, the unpaired CD3 specific VH or VL domain or the respective unpaired CyCAT polypeptides comprising the same should not activate T-cells and/or mediate redirected T-cell killing alone.

And finally, the unpaired CD3 specific VH or VL domain or the respective unpaired CyCAT polypeptides comprising the same should not pair before the two polypeptides bind to their respective cancer associated antigens on a target cell. Such unwanted pre-pairing (or hetero-association) may occur in patient serum after separate administration of the two polypeptides (e.g. as part of two pharmaceutical compositions) or when both polypeptides are part of one pharmaceutical composition. Unwanted pre-pairing is thought to be i.a. concentration dependent as well as dependent of the affinity of the splitted VH and VL domains to each other.

A plethora of antibodies binding to CD3 are described in the art. However, the majority of these antibodies with intended therapeutic use are derived (e.g. humanized) from a small set of rodent anti-CD3 antibodies discovered in the early 1980s and 1990s. Of these, SP34 (Yoshino N. et al., Exp. Anim 49:97-110, 2000; Conrad M L. et al., Cytometry 71A:925-33, 2007) is probably the most used antibody for humanization approaches because it is one of the few CD3 specific antibodies with cross-reactivity to cynomolgus monkey CD3 (cynomolgus CD3 or cyno CD3) and as such can be used both for preclinical evaluation of safety, activity and/or pharmacokinetic profile in primates and—in the identical form—as drugs in humans. SP34 recognizes an N-terminal 1-27 amino acid residue polypeptide fragment of the extracellular domain of human CD3 epsilon. However, the inventors of the present disclosure have found that at least three prior-art antibodies of which two are derived from SP34 were not suited to be used in the discussed CyCAT format based on their unfavorable production properties and/or functional activity.

Interestingly, de novo in vitro generation of fully human antibodies binding to CD3 recognizing the N-terminal extracellular region of human CD3 epsilon and thus being cross-reactive to cynomolgus CD3 appears challenging and has been at least to the knowledge of the present inventors not reported so far.

Accordingly, the present disclosure provides novel antibodies and antibody fragments specific for CD3 epsilon which are superior to the CD3 specific antibodies reported in the art.

In particular, the antibodies or antibody fragments specific for CD3 epsilon according to the present disclosure are fully human antibodies which also cross-react with cynomolgus CD3 with favorable affinity and combine desirable producibility, functionality and safety properties never observed before.

These features makes the antibodies and antibody fragments of the present disclosure highly desirable for therapeutic use in multispecific antibody formants, in particular in the CyCAT format as described herein.

### SUMMARY OF THE DISCLOSURE

Isolated human antibodies or antibody fragments as well as antibody variable domains specific for human CD3 epsilon and their respective CDRs according to the present disclosure as well as CyCAT polypeptides comprising the same are summarized in Tables 2-10 and Tables 17-18 of the present disclosure.

Preferred improved variable heavy chains (VH) and their HCDRs 1-3 according to the present disclosure are summarized in Table 4. Preferred improved variable light chains (VL) and their LCDRSs 1-3 according to the present disclosure are summarized in Table 5.

Preferred isolated human antibodies or antibody fragments specific for human CD3 epsilon according to the present disclosure are summarized in Table 10. Preferred combinations of improved variable heavy chains (VH) and improved variable light chains (VL) specific for human CD3 epsilon are summarized in Table 10. Most preferred isolated human antibodies or antibody fragments specific for human CD3 epsilon according to the present disclosure are summarized in Table 17. Most preferred combinations of improved variable heavy chains (VH) and improved variable light chains (VL) specific for human CD3 epsilon are summarized in Table 17.

In an embodiment, the present disclosure provides an isolated human antibody or antibody fragment specific for human CD3 epsilon comprising 6 CDRs disclosed in Table 2 and Table 3 of the present disclosure. In an embodiment, the present disclosure provides an isolated human antibody or antibody fragment specific for human CD3 epsilon comprising the 6 CDRs of an antibody disclosed in Table 2 or Table 3 of the present disclosure.

In an embodiment, the present disclosure provides an isolated human antibody or antibody fragment specific for human CD3 epsilon comprising the 3 HCDRs of an variable heavy chain (VH) disclosed in Table 4 and the 3 LCDRs of an antibody light chain (VL) disclosed in Table 5 of the present disclosure.

In an embodiment, the present disclosure provides an isolated human antibody or antibody fragment specific for human CD3 epsilon comprising the HCDRs of any one of the antibodies or variable domains disclosed in Table 4 and the LCDRs of any one of the antibodies or variable domains disclosed in Table 5 of the present disclosure.

In an embodiment, the present disclosure provides an isolated human antibody or antibody fragment specific for human CD3 epsilon comprising a combination of a variable heavy chain (VH) and a variable light chain (VL) as disclosed in Table 2, or Table 3 and Tables 8-10 of the present disclosure.

In an embodiment, the present disclosure provides an isolated human antibody or antibody fragment specific for human CD3 epsilon comprising a variable heavy chain (VH) and a variable light chain (VL) of any one of the antibodies disclosed in Table 2, 3 and Tables 8-10 of the present disclosure. In an embodiment, the present disclosure provides an isolated human antibody or antibody fragment specific for human CD3 epsilon comprising a variable heavy chain (VH) disclosed in Table 4 and a variable light chain (VL) disclosed in Table 5 of the present disclosure. In an embodiment, the present disclosure provides an isolated human antibody or antibody fragment specific for human CD3 epsilon comprising a combination of a variable heavy chain (VH) disclosed in Table 4 and a variable light chain (VL) disclosed in Table 5 of the present disclosure. In an embodiment, the present disclosure provides an isolated human antibody or antibody fragment specific for human CD3 epsilon disclosed in Table 10 of the present disclosure. In an embodiment, the present disclosure provides an isolated human antibody or antibody fragment specific for human CD3 epsilon disclosed in Table 17 of the present disclosure.

In an embodiment, the present disclosure provides an isolated human antibody or antibody fragment specific for human CD3 epsilon comprising a combination of a variable heavy chain (VH) disclosed and a variable light chain (VL) disclosed in Table 10 of the present disclosure.

In an embodiment, the present disclosure provides an isolated human antibody or antibody fragment specific for human CD3 epsilon, wherein said antibody or antibody fragment comprises:
a) an HCDR1 region comprising the amino acid sequence of GFTFX₁SX₂X₃MX₄, wherein X₁ is S, K or R; X₂ is Y or H; X₃ is W or Y; and X₄ is S or T (SEQ ID NO: 90);
b) an HCDR2 region comprising the amino acid sequence of NIX₁X₂X₃X₄X₅X₆X₇YYX₈X₉SVKG, wherein X₁ is K or D; X₂ is Q or Y; X₃ is D, Q or E; X₄ is S or G; X₅ is S, Q or T; X₆ is E, H or R; X₇ is K, A or T; X₈ is V or A; and X₉ is D or E (SEQ ID NO: 91);
c) an HCDR3 region comprising the amino acid sequence of GYSAEFAHRSGLDV (SEQ ID NO: 39);
d) a LCDR1 region comprising the amino acid sequence of SGSSSNIGXX₂YVY, wherein X₁ is S, I, A, K or Q; and X₂ is N or T (SEQ ID NO: 92);
e) a LCDR2 region comprising the amino acid sequence of RNX₁X₂RPS, wherein X₁ is N, K, S, H, T or Y; and X₂ is Q, I or K (SEQ ID NO: 93); and
f) a LCDR3 region comprising the amino acid sequence of AX₁WDX₂X₃X₄X₅GAV, wherein X₁ is A or G; X₂ is H or R; X₃ is H or R; X₄ is R, S or L; and X₅ is S or H (SEQ ID NO: 94).

In an embodiment, the present disclosure provides an isolated human antibody or antibody fragment specific for human CD3 epsilon, wherein said antibody or antibody fragment comprises:
a) an HCDR1 region comprising the amino acid sequence of GFTFSSYWMS (SEQ ID NO: 40), GFTFKSYYMS (SEQ ID NO: 41) or GFTFRSHYMT (SEQ ID NO: 42); b) an HCDR2 region comprising the amino acid sequence of NIKQDGSEKYYVDSVKG (SEQ ID NO: 43), NIDYQSQHAYYAESVKG (SEQ ID NO: 44) or NIDYEGTRTYYAESVKG (SEQ ID NO: 45);
c) an HCDR3 region comprising the amino acid sequence of GYSAEFAHRSGLDV (SEQ ID NO: 39);
d) a LCDR1 region comprising the amino acid sequence of SGSSSNIGSNYVY (SEQ ID NO: 46), SGSSSNIGINYVY (SEQ ID NO: 47), SGSSSNIGANYVY (SEQ ID NO: 48), SGSSSNIGQTYVY (SEQ ID NO: 49), or SGSSSNIGKNYVY (SEQ ID NO: 50);
e) a LCDR2 region comprising the amino acid sequence of RNNQRPS (SEQ ID NO: 51), RNNIRPS (SEQ ID NO: 52), RNNKRPS (SEQ ID NO: 53), RNKKRPS (SEQ ID NO: 54), RNKQRPS (SEQ ID NO: 55), RNSQRPS (SEQ ID NO: 56), RNHIRPS (SEQ ID NO: 57), RNTQRPS (SEQ ID NO: 58), or RNYQRPS (SEQ ID NO: 59); and
f) a LCDR3 region comprising the amino acid sequence of AAWDHHRSGAV (SEQ ID NO: 60), AAWDRHSHGAV (SEQ ID NO: 61) or AGWDRRLHGAV (SEQ ID NO: 62).

In an embodiment, the present disclosure provides an isolated human antibody or antibody fragment specific for human CD3 epsilon, wherein said antibody or antibody fragment comprises a variable heavy chain (VH) comprising the amino acid sequence of SEQ ID NO: 13, SEQ ID NO: 14, or SEQ ID NO: 15.

In an embodiment, the present disclosure provides an isolated human antibody or antibody fragment specific for human CD3 epsilon, wherein said antibody or antibody fragment comprises a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure provides an isolated human antibody or antibody fragment specific for human CD3 epsilon, wherein said antibody or antibody fragment comprises
a) a variable heavy chain (VH) comprising the amino acid sequence of SEQ ID NO: 13, SEQ ID NO: 14, or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure provides an isolated human antibody or antibody fragment specific for human CD3 epsilon, wherein said antibody or antibody fragment comprises a variable heavy chain (VH) and a variable light chain (VH) selected from the group consisting of:
a) the VH comprising the amino acid sequence of SEQ ID NO: 13 and the VL comprising the amino acid sequence of SEQ ID NO: 16,
b) the VH comprising the amino acid sequence of SEQ ID NO: 14 and the VL comprising the amino acid sequence of SEQ ID NO: 16,
c) the VH comprising the amino acid sequence of SEQ ID NO: 13 and the VL comprising the amino acid sequence of SEQ ID NO: 17,
d) the VH comprising the amino acid sequence of SEQ ID NO: 14 and the VL comprising the amino acid sequence of SEQ ID NO: 17,
e) the VH comprising the amino acid sequence of SEQ ID NO: 14 and the VL comprising the amino acid sequence of SEQ ID NO: 18,
f) the VH comprising the amino acid sequence of SEQ ID NO: 14 and the VL comprising the amino acid sequence of SEQ ID NO: 19,
g) the VH comprising the amino acid sequence of SEQ ID NO: 14 and the VL comprising the amino acid sequence of SEQ ID NO: 20,
h) the VH comprising the amino acid sequence of SEQ ID NO: 14 and the VL comprising the amino acid sequence of SEQ ID NO: 21,
i) the VH comprising the amino acid sequence of SEQ ID NO: 14 and the VL comprising the amino acid sequence of SEQ ID NO: 22,
j) the VH comprising the amino acid sequence of SEQ ID NO: 14 and the VL comprising the amino acid sequence of SEQ ID NO: 23,
k) the VH comprising the amino acid sequence of SEQ ID NO: 14 and the VL comprising the amino acid sequence of SEQ ID NO: 24,
l) the VH comprising the amino acid sequence of SEQ ID NO: 14 and the VL comprising the amino acid sequence of SEQ ID NO: 25,
m) the VH comprising the amino acid sequence of SEQ ID NO: 14 and the VL comprising the amino acid sequence of SEQ ID NO: 26,
n) the VH comprising the amino acid sequence of SEQ ID NO: 14 and the VL comprising the amino acid sequence of SEQ ID NO: 27,
o) the VH comprising the amino acid sequence of SEQ ID NO: 14 and the VL comprising the amino acid sequence of SEQ ID NO: 28,
p) the VH comprising the amino acid sequence of SEQ ID NO: 14 and the VL comprising the amino acid sequence of SEQ ID NO: 29,
q) the VH comprising the amino acid sequence of SEQ ID NO: 14 and the VL comprising the amino acid sequence of SEQ ID NO: 30,
r) the VH comprising the amino acid sequence of SEQ ID NO: 14 and the VL comprising the amino acid sequence of SEQ ID NO: 31,
s) the VH comprising the amino acid sequence of SEQ ID NO: 14 and the VL comprising the amino acid sequence of SEQ ID NO: 32,
t) the VH comprising the amino acid sequence of SEQ ID NO: 14 and the VL comprising the amino acid sequence of SEQ ID NO: 33,
u) the VH comprising the amino acid sequence of SEQ ID NO: 14 and the VL comprising the amino acid sequence of SEQ ID NO: 34,
v) the VH comprising the amino acid sequence of SEQ ID NO: 14 and the VL comprising the amino acid sequence of SEQ ID NO: 35,
w) the VH comprising the amino acid sequence of SEQ ID NO: 14 and the VL comprising the amino acid sequence of SEQ ID NO: 36,
x) the VH comprising the amino acid sequence of SEQ ID NO: 14 and the VL comprising the amino acid sequence of SEQ ID NO: 37,
y) the VH comprising the amino acid sequence of SEQ ID NO: 14 and the VL comprising the amino acid sequence of SEQ ID NO: 38,
z) the VH comprising the amino acid sequence of SEQ ID NO: 15 and the VL comprising the amino acid sequence of SEQ ID NO: 17,
aa) the VH comprising the amino acid sequence of SEQ ID NO: 15 and the VL comprising the amino acid sequence of SEQ ID NO: 18,
bb) the VH comprising the amino acid sequence of SEQ ID NO: 15 and the VL comprising the amino acid sequence of SEQ ID NO: 19,
cc) the VH comprising the amino acid sequence of SEQ ID NO: 15 and the VL comprising the amino acid sequence of SEQ ID NO: 20,
dd) the VH comprising the amino acid sequence of SEQ ID NO: 15 and the VL comprising the amino acid sequence of SEQ ID NO: 21,
ee) the VH comprising the amino acid sequence of SEQ ID NO: 15 and the VL comprising an amino acid sequence of SEQ ID NO: 22,
ff) the VH comprising the amino acid sequence of SEQ ID NO: 15 and the VL comprising the amino acid sequence of SEQ ID NO: 23,
gg) the VH comprising the amino acid sequence of SEQ ID NO: 15 and the VL comprising the amino acid sequence of SEQ ID NO: 24,
hh) the VH comprising the amino acid sequence of SEQ ID NO: 15 and the VL comprising the amino acid sequence of SEQ ID NO: 25,
ii) the VH comprising the amino acid sequence of SEQ ID NO: 15 and the VL comprising the amino acid sequence of SEQ ID NO: 26,
jj) the VH comprising the amino acid sequence of SEQ ID NO: 15 and the VL comprising the amino acid sequence of SEQ ID NO: 27,
kk) the VH comprising the amino acid sequence of SEQ ID NO: 15 and the VL comprising the amino acid sequence of SEQ ID NO: 28,
ll) the VH comprising the amino acid sequence of SEQ ID NO: 15 and the VL comprising the amino acid sequence of SEQ ID NO: 29,
mm) the VH comprising the amino acid sequence of SEQ ID NO: 15 and the VL comprising the amino acid sequence of SEQ ID NO: 30,
nn) the VH comprising the amino acid sequence of SEQ ID NO: 15 and the VL comprising the amino acid sequence of SEQ ID NO: 31,
oo) the VH comprising the amino acid sequence of SEQ ID NO: 15 and the VL comprising the amino acid sequence of SEQ ID NO: 32,
pp) the VH comprising the amino acid sequence of SEQ ID NO: 15 and the VL comprising the amino acid sequence of SEQ ID NO: 33,
qq) the VH comprising the amino acid sequence of SEQ ID NO: 15 and the VL comprising the amino acid sequence of SEQ ID NO: 34,
rr) the VH comprising the amino acid sequence of SEQ ID NO: 15 and the VL comprising the amino acid sequence of SEQ ID NO: 35,
ss) the VH comprising the amino acid sequence of SEQ ID NO: 15 and the VL comprising the amino acid sequence of SEQ ID NO: 36,
tt) the VH comprising the amino acid sequence of SEQ ID NO: 15 and the VL comprising the amino acid sequence of SEQ ID NO: 37, and
uu) the VH comprising the amino acid sequence of SEQ ID NO: 15 and the VL comprising the amino acid sequence of SEQ ID NO: 38.

In an embodiment, the isolated human antibody or antibody fragment specific for human CD3 epsilon according to the present disclosure cross-reactively binds to cynomolgus CD3 epsilon. In an embodiment, the isolated human antibody or antibody fragment specific for human CD3 epsilon according to the present disclosure is cross-reactive to cynomolgus CD3 epsilon.

In an embodiment, the isolated human antibody or antibody fragment specific for human CD3 epsilon according to the present disclosure is a recombinant antibody or antibody fragment. In an embodiment, the isolated human antibody or antibody fragment specific for human CD3 epsilon according to the present disclosure is a monoclonal antibody or antibody fragment.

In an embodiment, the isolated human antibody or antibody fragment specific for human CD3 epsilon according to the present disclosure is an antibody fragment selected from the group consisting of Fab, Fv, and scFv. In an embodiment, the isolated human antibody or antibody fragment specific for human CD3 epsilon according to the present disclosure is a Fab fragment. In an embodiment, the isolated human antibody or antibody fragment specific for human CD3 epsilon according to the present disclosure is a single chain Fv (scFv) fragment. In an embodiment, the isolated antibody or antibody fragment specific for human CD3 epsilon according to the present disclosure is a Fv domain. In an embodiment, the isolated human antibody or antibody fragment specific for human CD3 epsilon according to the present disclosure is an IgG. In an embodiment, the isolated human antibody or antibody fragment specific for human CD3 epsilon according to the present disclosure is an full-length IgG. In an embodiment, said isolated human antibody or antibody fragment specific for human CD3 epsilon according to the present disclosure is an full-length IgG of an isotype selected from the group consisting of IgG1, IgG2, IgG3, and IgG4.

In an embodiment, said isolated human antibody or antibody fragment specific for human CD3 epsilon according to the present disclosure is an IgG comprising an Fc region that has reduced effector function relative to that of a wild type Fc-receptor. “Effector function” as used in this context refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

In an embodiment, said isolated human antibody or antibody fragment specific for human CD3 epsilon, comprises a Fc region, wherein in at least 5 amino acids in the positions corresponding to positions L234, L235, D237, N330, P331 in a human IgG1 heavy chain, are mutated to A, E, A, S, and S, respectively. In an embodiment, said isolated human antibody or antibody fragment specific for human CD3 epsilon, comprises a Fc region, wherein in at least 5 amino acids in the positions corresponding to positions L234, L235, G237, N330, P331 in a human IgG1 heavy chain, are mutated to A, E, A, S, and S, respectively.

In an embodiment, the present disclosure provides a multispecific antibody comprising the isolated human antibody or antibody fragment specific for human CD3 epsilon according to the present disclosure. In an embodiment of the present disclosure, said multispecific antibody also specifically binds to a cancer associated antigen.

In an embodiment, the present disclosure provides a multispecific antibody comprising the isolated human antibody or antibody fragment specific for human CD3 epsilon according to the present disclosure and a second antibody or antibody fragment which binds to a different antigen than the antibody or antibody fragment specific for human CD3 epsilon according to the present disclosure. In an embodiment, said second antibody fragment binds to a cell surface target antigen. In an embodiment, said cell surface target antigen is a tumor antigen. In an embodiment, said second antibody fragment binds to a cancer associated antigen on a cancer cell. In an embodiment, the present disclosure provides a multispecific antibody comprising a first antibody fragment of the human antibody or antibody fragment specific for human CD3 epsilon according to the present disclosure and a second antibody fragment which binds to a different antigen than said first antibody fragment. In an embodiment, said multispecific antibody comprises a third antibody fragment which binds to a different antigen than said first and second antibody fragment. In an embodiment, said multispecific antibody comprises a third antibody fragment which binds to the same antigen than said second antibody fragment. In an embodiment, said second and third antibody fragment binds to a cell surface target antigen. In an embodiment, said cell surface target antigen is a tumor antigen. In an embodiment, said second and third antibody fragment binds to a cancer associated antigen on a cancer cell.

In an embodiment, the present disclosure provides a nucleic acid composition comprising a nucleic acid sequence or a plurality of nucleic acid sequences encoding the human antibody or antibody fragment specific for human CD3 epsilon according to the present disclosure or the multispecific antibody according to the present disclosure. In an embodiment, the present disclosure provides a vector composition comprising a vector or a plurality of vectors comprising the nucleic acid sequence or plurality of nucleic acid sequences according to the present disclosure. In an embodiment, the present disclosure provides a host cell comprising the vector composition according to the present disclosure. In an embodiment, said host cell is mammalian cell. In an embodiment, said host cell is prokaryotic cell. In another embodiment, the disclosure provides a method of producing an isolated human antibody or antibody fragment specific for human CD3 epsilon according to the present disclosure.

In an embodiment, the present disclosure pertains to an isolated human antibody or antibody fragment specific for human CD3 epsilon according to the present disclosure or the multispecific antibody according to the present disclosure for use as a medicament. In further embodiments, the present disclosure provides an isolated human antibody or antibody fragment specific for CD3 epsilon according to the present disclosure for use in the treatment of a subject in need thereof.

In an embodiment, the present disclosure also provides methods for treating patients suffering from a disease, such as an proliferative disease, by administering to said patient an effective amount of an isolated human antibody or antibody fragment specific for human CD3 epsilon according to the present disclosure. In an embodiment, the present disclosure provides a pharmaceutical composition comprising the human antibody or antibody fragment specific for CD3 epsilon according to the present disclosure or the multispecific antibody according to the present disclosure and a pharmaceutically acceptable carrier or excipient.

In an embodiment, the present disclosure provides a method for re-directing cytotoxic activity of a T cell to a cancer cell comprising contacting said cancer cell in the presence of a T cell with the multispecific antibody according to the present disclosure.

There is utility in the claimed antibodies or antibody fragments. Furthermore, there is utility in the claimed method to generate such antibodies or antibody fragments. Utilization of the claimed antibodies or antibody fragments is to target T cells expressing CD3, and for stimulating T cell activation, e.g. under circumstances where T cell mediated killing is beneficial or desirable. In particular the claimed antibodies or antibody fragments are for therapeutic use, such as the treatment of cancer.

### FIGURE LEGENDS

FIG. 1: Cell binding of mammalian produced CD3 specific MAB-1 IgG. FIG. 1A shows binding as a function of IgG concentration as determined on human derived PBMCs expressing human CD3 by flow cytometry. As negative control, an IgG against hen egg lysozyme was included. FIG. 1B depicts the same as FIG. 1A with the difference that binding on cynomolgus monkey derived PBMCs expressing cynomolgus CD3 is shown.

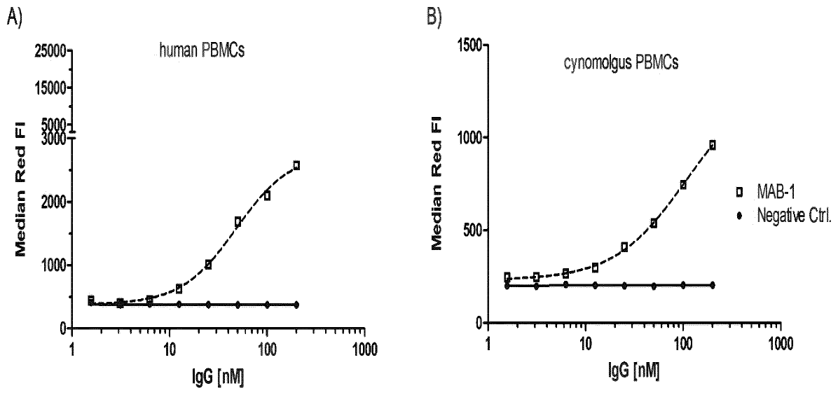

FIG. 2: Schematic principle of the CyCAT format: A set of polypeptides is designed, each composed of an targeting moiety (e.g. antibody Fab fragment) fused to either the variable light chain domain (VL) or the complementary variable heavy chain (VH) domain of a T cell activating anti-CD3 antibody. The unpaired VH or VL domain are not able to bind to CD3. Once the aforementioned two polypeptides bind to their antigen on the surface of a cell via their targeting moiety, the complementary VL and VH domains come in close proximity and interact with each other to reconstitute the original CD3 antibody fragment (e.g. the CD3 specific Fv domain). The thus on-target cell formed trispecific heterodimeric molecule engages and stimulates T cells for tumor cell destruction such as a conventional trivalent bi- or trispecifc antibody format.

### DETAILED DESCRIPTION OF THE DISCLOSURE

### Definitions

The term “CD3” refers to an antigen which is expressed on T cells as part of the multi-molecular T cell receptor (TCR) and which consists of a homodimer or heterodimer formed from the association of two of four receptor chains: CD3-epsilon, CD3-delta, CD3-zeta, and CD3-gamma.

Human CD3 epsilon including signal sequence (underlined) has the amino acid sequence of UniProt P07766:

(SEQ ID NO: 1)
MQSGTHWRVLGLCLLSVGVWGQDGNEEMGGITQTPYKVSISG
TTVILTCPQYPGSEILWQHNDKNIGGDEDDKNIGSDEDHLSL
KEFSELEQSGYYVCYPRGSKPEDANFYLYLRARVCENCMEMD
VMSVATIVIVDICITGGLLLLVYYWSKNRKAKAKPVTRGAGA
GGRQRGQNKERPPPVPNPDYEPIRKGQRDLYSGLNQRRI

Cynomolgus CD3 epsilon including signal sequence (underlined) has the amino acid sequence of UniProt Q95L15:

(SEQ ID NO: 2)
MQSGTRWRVLGLCLLSIGVWGQDGNEEMGSITQTPYQVSISGT
TVILTCSQHLGSEAQWQHNGKNKEDSGDRLFLPEFSEMEQSGY
YVCYPRGSNPEDASHHLYLKARVCENCMEMDVMAVATIVIVDI
CITLGLLLLVYYWSKNRKAKAKPVTRGAGAGGRQRGQNKERPP
PVPNPDYEPIRKGQQDLYSGLNQRRI

The terms “antigen” or “target antigen” as used herein refers to any molecule of interest that can be bound by one of the binding sites present in an antigen binding molecule according to the present disclosure. Typically, an antigen is a peptide, a protein or any other proteinaceous molecule. Alternatively, an antigen may be any other organic or inorganic molecule, such as carbohydrate, fatty acid, lipid, dye or flourophor.

The term “antigen binding molecule” as used herein, refers in its broadest sense to a proteinaceous molecule that specifically binds to at least one antigen. An antigen binding molecule may be composed of one or more polypeptides. Examples of antigen binding molecules are immunoglobulins and derivatives and/or fragments thereof. Antigen binding molecules according to the present disclosure may be based on a regular immunoglobulin (e.g. IgG), in particular of half IgG molecules. The antigen binding molecule as disclosed herein are composed of at least a targeting moiety (such as an antibody Fab fragment) and an additional VH or VL domain of an antibody Fv domain wherein neither the VH or VL domain is able to bind to its antigen alone. Accordingly, an antigen binding molecule according to the preset disclosure incorporates a half Fv domain (½ Fv domain) or a half binding site and a full binding site and thus can be also denoted as a 1+½ or 1½ antigen binding molecule.

The term “antibody” as used herein refers to a protein comprising at least two heavy (H) chains and two light (L) chains inter-connected by disulfide bonds which interacts with an antigen. Each heavy chain is comprised of a heavy chain variable region or domain (abbreviated herein as VH) and a heavy chain constant region. The heavy chain constant region is comprised of three domains, CH1, CH2 and CH3. Each light chain is comprised of a light chain variable region or domain (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment”, as used herein, refers to one or more portions of an antibody that retain the ability to specifically interact with (e.g., by binding, steric hindrance, stabilizing spatial distribution) an antigen. Examples of antibody fragments include, but are not limited to, a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment (or Fv domain) consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment (or Fv domain), VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

A “human antibody” or “human antibody fragment”, as used herein, includes antibodies and antibody fragments having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86). Thereby said human antibody can be obtained from technology platforms which comprise antibodies derived from human germline genes either generated by PCR-amplification of VHA/L repertoire isolated from B-cells or are generated synthetically. Technology platforms include library based approaches comprising human immunoglobulin genes displayed on phage, ribosome or yeast. Respective display technologies are standard in the scientific community. Furthermore immunization of a transgenic mouse carrying human immunoglobulin repertoire is another approach to generate human antibodies against an antigen of interest. Antibodies or fragments thereof selected from an antibody library based on the MorphoSys HuCAL® concept (Knappik et al., (2000) J Mol Biol 296:57-86) as well as the from the MorphoSys Ylanthia® library (Tiller et al. mAbs 5:3, 1-26; May/June (2013) and U.S. Pat. No. 8,728,981) are considered as fully human.

A “humanized antibody” or “humanized antibody fragment” is defined herein as an antibody or antibody fragment which has constant antibody regions derived from sequences of human origin and the variable antibody regions or parts thereof or only the CDRs are derived from another species. For example a humanized antibody can be CDR-grafted, wherein the CDRs of the variable domain are from a non-human origin, while one or more frameworks of the variable domain are of human origin and the constant domain (if any) is of human origin.

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and AI-Lazikani et al., (1997) J. Mol. Biol. 273:927-948.

The term “isolated” refers to a compound, which can be e.g. an antibody or antibody fragment, that is substantially free of other antibodies or antibody fragments having different antigenic specificities. Thus, in some aspects, antibodies provided are isolated antibodies which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody”, as used herein, includes all antibodies that are prepared, expressed, created or segregated by means not existing in nature. For example antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody may be a monoclonal antibody.

As used herein, the term “monoclonal antibody” refers to an antibody that is derived from a single clone, including any eukaryotic, prokaryotic, or phage clone, and not the method by which it is produced. Monoclonal antibodies as disclosed herein may be made by the hybridoma method as described in Kohler et al.; Nature, 256:495 (1975) or may be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Other exemplary methods of producing other monoclonal antibodies are provided in the Examples herein.

As used herein, an antibody “binds specifically to”, “specifically binds to”, is “specific to/for” or “specifically recognizes” an antigen if such antibody is able to discriminate between such antigen and one or more reference antigen(s), since binding specificity is not an absolute, but a relative property. For example, a standard ELISA assay can be carried out. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

As used herein, “an antibody that binds to CD3” or an “anti-CD3 antibody” or “antibody specific for CD3” includes antibodies and antibody fragments that specifically recognize one or more CD3 receptor chain (or subunits) (e.g., epsilon, delta, gamma or zeta), as well as antibodies and antibody fragments that specifically recognize a dimeric complex of two CD3 receptor chains (e.g., gamma/epsilon, delta/epsilon, and zeta/zeta CD3 dimers). The antibodies and antibody fragments of the present disclosure may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane. Use of the term “antibody that binds to CD3” or an “anti-CD3 antibody” or a “CD3 specific antibody” herein is intended to include both, monospecific antibodies as well as bispecific or multispecific antibodies.

“Cell surface-expressed CD3” includes CD3 proteins contained within the context of a functional T cell receptor in the membrane of a cell. The expression “cell surface-expressed CD3” also includes CD3 protein expressed as part of a homodimer or heterodimer on the surface of a cell (e.g., gamma/epsilon, delta/epsilon, and zeta/zeta CD3 receptor chain dimers). The expression, “cell surface-expressed CD3” also includes a CD3 receptor chain (e.g., CD3-epsilon, CD3-delta or CD3-gamma or CD3-zeta) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

The term “cross-reactively binds” or the term “cross-reactive” are used herein interchangeably and refers to an antibody which has the ability to specifically bind to more than one antigen. For example, in the present disclosure the antibody cross-reactively binds to cynomolgus CD3 or cynomolgus CD3 epsilon.

As used herein, the term “affinity” refers to the strength of interaction between the polypeptide and its target at a single site. Within each site, the binding region of the polypeptide interacts through weak non-covalent forces with its target at numerous sites; the more interactions, the stronger the affinity. The term “K_{D}”, as used herein, refers to the dissociation constant, which is obtained from the ratio of K_{d} to Kₐ (i.e. K_{d}/Kₐ) and is expressed as a molar concentration (M). K_{D} values for antigen binding moieties like e.g. monoclonal antibodies can be determined using methods well established in the art. Methods for determining the K_{D} of an antigen binding moiety like e.g. a monoclonal antibody are SET (soluble equilibrium titration) or surface plasmon resonance using a biosensor system such as a Biacore® system. In the present disclosure an antibody specific to the human CD3 epsilon polypeptide typically has a dissociation rate constant (K_{D}) (k_{off}/kₒₙ) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻¹M, less than 10⁻⁸M, less than 5×10⁻⁹M, less than 10⁻⁹M, less than 5×10⁻¹⁰ M, less than 10⁻¹⁰M, less than 5×10⁻¹¹M, less than 10⁻¹¹M, less than 5×10¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁸M or lower.

Compositions of the present disclosure may be used for therapeutic or prophylactic applications. The present disclosure, therefore, includes a pharmaceutical composition containing an antibody (or functional antibody fragment) as disclosed herein and a pharmaceutically acceptable carrier or excipient therefor. In a related aspect, the present disclosure provides a method for treating cancer. Such method contains the steps of administering to a subject in need thereof an effective amount of the pharmaceutical composition that contains an antibody (or functional antibody fragment) as described herein.

The present disclosure provides therapeutic methods comprising the administration of a therapeutically effective amount of a human antibody or antibody fragment specific for CD3 as disclosed herein to a subject in need of such treatment. A “therapeutically effective amount” or “effective amount”, as used herein, refers to the amount of an CD3 specific antibody necessary to elicit the desired biological response. In accordance with the disclosure, the therapeutic effective amount is the amount of a CD3 specific antibody or antibody fragment necessary to treat and/or prevent a disease.

The terms “cell proliferative disease” and “proliferative disease” refer to diseases that are associated with some degree of abnormal cell proliferation. In one embodiment, the cell proliferative disease is cancer. In one embodiment, the cell proliferative disease is a tumor.

The terms “cancer” and “cancerous” refer to or describe the physiological condition in mammals that is typically characterized by unregulated cell growth.

As used herein, “delaying progression” of a disease or disease means to defer, hinder, slow, retard, stabilize, and/or postpone development of the disease or disease (e.g., a cell proliferative disease, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

The term “IC₅₀”, as used herein, refers to the concentration of an inhibitor (e.g. an antibody or antibody fragment) that inhibits a response in an assay half way between the maximal response and the baseline. It represents the antibody concentration that reduces a given response by 50%.

The terms “inhibition” or “inhibit” or “reduction” or “reduce” or “neutralization” or “neutralize” refer to a decrease or cessation of any phenotypic characteristic (such as binding, a biological activity or function) or to the decrease or cessation in the incidence, degree, or likelihood of that characteristic. The “inhibition”, “reduction” or “neutralization” needs not to be complete as long as it is detectable using an appropriate assay. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

As used herein, “treatment”, “treat” or “treating” and the like refers to clinical intervention in an attempt to alter the natural course of a disease in the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies or antibody fragments according to the preset disclosure are used to delay development of a disease or to slow the progression of a disease.

The term “multispecific” means that an antibody is able to specifically bind to two or more different antigens. Typically, a multispecific antigen-binding molecule comprises of two or more antigen-binding sites, each of which is specific for a different antigen or epitope.

The term “bispecific” means that an antibody is able to specifically bind to two different antigens. Typically, a bispecific antigen-binding molecule comprises two antigen-binding sites, each of which is specific for a different antigen or epitope.

As used herein, the terms “first” and “second” with respect to a Fab and/or Fv region or the like are used for distinguishing when there is more than one of each type of component. Use of these terms is not intended to confer a specific order or orientation unless explicitly so stated.

As used herein, “amino acid residues” or “amino acid” will be indicated either by their full name or according to the standard three-letter or one-letter amino acid code. “Natural occurring amino acids” means the following amino acids:

TABLE 1
Natural occurring amino acids

| Amino acid | Three letter code | One letter code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “polypeptide” as used herein refer to a polymer of amino acid residues and does not refer to a specific length of a product. The term applies to naturally occurring amino acid polymers and non-naturally occurring amino acid polymers. Unless otherwise indicated, a particular amino acid sequence of a polypeptide also implicitly encompasses conservatively modified variants thereof (e.g. by replacing an amino acid residue with another amino acid residue having similar structural and/or chemical properties). A polypeptide may be derived from a natural biological source or produced by recombinant technology, but is not necessarily translated from a designated nucleic acid sequence. It may be generated in any manner, including chemical synthesis. A polypeptide may include one or more disulfide bonds or be chemically modified.

The term “domain”, as used herein, refers to a linear molecular chain of amino acids that includes the amino acid sequence of an entire polypeptide or a portion of a polypeptide

A “functional domain”, as used herein in the context of a CyCAT molecule according to the present disclosure, is a domain that is capable of fulfilling a certain function, such as specific binding to a certain binding partner or antigen, specific activation of a certain receptor, mediation of toxic effects, or fluorescence upon excitation with light of an appropriate wavelength.

The term “a fragment of a domain” or ““a fragment of a functional domain”, as used herein in the context of a CyCAT molecule according to the present disclosure, refers to a linear molecular chain of amino acids that corresponds to a part of a domain, but not the entire domain or functional domain.

The term “moiety”, as used herein in the context of a CyCAT molecule according to the present disclosure, refers to a linear molecular chain of amino acids that includes the amino acid sequence of an entire polypeptide or a portion of a polypeptide. A “targeting moiety” is used to refer to a moiety that specifically binds to an antigen. Non-limiting examples of targeting moieties are antibodies, antibody fragments, a natural ligand of an antigen, a mutein of such ligand or a mimetic if such ligand,

“Not associated” in the context of a CyCAT molecule according to present disclosure means not functionally associated with respect of the function of a functional domain, i.e. not allowing a first fragment of said functional domain and a second fragment of said functional domain to form said functional domain; or with respect of the function of the Fv domain, i.e. not allowing the VH and VL to form a functional Fv domain.

As used herein, “non-covalent association” refers to molecular interactions that do not involve an interatomic bond. Noncovalent interactions involve, for example, ionic bonds, hydrogen bonds, hydrophobic interactions, and van der Waals forces.

As used herein, “covalent bond” refers to an interatomic bond characterized by sharing of electrons.

### EMBODIMENTS

In an embodiment, the present disclosure provides an isolated human antibody or antibody fragment specific for human CD3 epsilon, wherein said antibody or antibody fragment comprises:
a) an HCDR1 region comprising the amino acid sequence of GFTFX₁SX₂X₃MX₄, wherein X₁ is S, K or R; X₂ is Y or H; X₃ is W or Y; and X₄ is S or T (SEQ ID NO: 90);
b) an HCDR2 region comprising the amino acid sequence of NIX₁X₂X₃X₄X₅X₆X₇YYX₈X₉SVKG, wherein X₁ is K or D; X₂ is Q or Y; X₃ is D, Q or E; X₄ is S or G; X₅ is S, Q or T; X₆ is E, H or R; Xy is K, A or T; X₈ is V or A; and X₉ is D or E (SEQ ID NO: 91);
c) an HCDR3 region comprising the amino acid sequence of GYSAEFAHRSGLDV (SEQ ID NO: 39);
d) a LCDR1 region comprising the amino acid sequence of SGSSSNIGX₁X₂YVY, wherein X₁ is S, I, A, K or Q; and X₂ is N or T (SEQ ID NO: 92);
e) a LCDR2 region comprising the amino acid sequence of RNX₁X₂RPS, wherein X₁ is N, K, S, H, T or Y; and X₂ is Q, I or K (SEQ ID NO: 93); and
f) a LCDR3 region comprising the amino acid sequence of AX₁WDX₂X₃X₄X₅GAV, wherein X₁ is A or G; X₂ is H or R; X₃ is H or R; X₄ is R, S or L; and X₅ is S or H (SEQ ID NO: 94).

In an embodiment, the present disclosure provides an isolated human antibody or antibody fragment specific for human CD3 epsilon, wherein said antibody or antibody fragment comprises:
a) an HCDR1 region comprising the amino acid sequence of GFTFSSYWMS (SEQ ID NO: 40), GFTFKSYYMS (SEQ ID NO: 41) or GFTFRSHYMT (SEQ ID NO: 42);
b) an HCDR2 region comprising the amino acid sequence of NIKQDGSEKYYVDSVKG (SEQ ID NO: 43), NIDYQSQHAYYAESVKG (SEQ ID NO: 44) or NIDYEGTRTYYAESVKG (SEQ ID NO: 45);
c) an HCDR3 region comprising the amino acid sequence of GYSAEFAHRSGLDV (SEQ ID NO: 39);
d) a LCDR1 region comprising the amino acid sequence of SGSSSNIGSNYVY (SEQ ID NO: 46), SGSSSNIGINYVY (SEQ ID NO: 47) SGSSSNIGANYVY (SEQ ID NO: 48), SGSSSNIGQTYVY (SEQ ID NO: 49), or SGSSSNIGKNYVY (SEQ ID NO: 50);
e) a LCDR2 region comprising the amino acid sequence of RNNQRPS (SEQ ID NO: 51), RNNIRPS (SEQ ID NO: 52), RNNKRPS (SEQ ID NO: 53), RNKKRPS (SEQ ID NO: 54), RNKQRPS (SEQ ID NO: 55), RNSQRPS (SEQ ID NO: 56), RNHIRPS (SEQ ID NO: 57), RNTQRPS (SEQ ID NO: 58), or RNYQRPS (SEQ ID NO: 59); and
f) a LCDR3 region comprising the amino acid sequence of AAWDHHRSGAV (SEQ ID NO: 60), AAWDRHSHGAV (SEQ ID NO: 61) orAGWDRRLHGAV (SEQ ID NO: 62).

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 40;
b) an HCDR2 region comprising the amino acid sequence of SEQ ID NO: 43;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 60.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 60.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 40;
b) an HCDR2 region comprising the amino acid sequence of SEQ ID NO: 43;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 52; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 53; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 54; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 55; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 56; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 57; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 51; and
f) a LCDR3 region comprising the amino acid sequence of SEQ ID NO: 61

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 55; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 58; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 50;
e) a LCDR2 region comprising the amino acid sequence of SEQ ID NO: 59; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 58; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 53; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 57; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 55; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 58; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 58; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 41;
b) an HCDR2 region comprising the amino acid sequence of SEQ ID NO: 44;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 52; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 52; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 53; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 54; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 55; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 56; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 57; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 51; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 55; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 58; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 50;
e) a LCDR2 region comprising the amino acid sequence of SEQ ID NO: 59; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 46;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 58; and
f) a LCDR3 region comprising the amino acid sequence of SEQ ID NO: 61.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 53; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 57; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 51; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 48;
e) a LCDR2 region comprising the amino acid sequence of SEQ ID NO: 55; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 58; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 49;
e) a LCDR2 region comprising the amino acid sequence of SEQ ID NO: 58; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises:
a) an HCDR1 region comprising the amino acid sequence of SEQ ID NO: 42;
b) an HCDR2 region comprising the amino acid sequence of SEQ ID NO: 45;
c) an HCDR3 region comprising the amino acid sequence of SEQ ID NO: 39;
d) a LCDR1 region comprising the amino acid sequence of SEQ ID NO: 47;
e) a LCDR2 region comprising the amino acid sequence of SEQ ID NO: 52; and
f) a LCDR3 region comprising the amino acid sequence of SEQ ID NO: 62.

In an embodiment, the present disclosure provides an isolated human antibody or antibody fragment specific for human CD3 epsilon, wherein said antibody or antibody fragment comprises a variable heavy chain (VH) comprising the amino acid sequence of SEQ ID NO: 13, SEQ ID NO: 14 or SEQ ID NO: 15.

In an embodiment, the present disclosure provides an isolated human antibody or antibody fragment specific for human CD3 epsilon, wherein said antibody or antibody fragment comprises a variable heavy chain (VH) comprising the amino acid sequence of SEQ ID NO: 14 or SEQ ID NO: 15.

In an embodiment, the present disclosure provides an isolated human antibody or antibody fragment specific for human CD3 epsilon, wherein said antibody or antibody fragment comprises a variable heavy chain (VH) comprising the amino acid sequence of SEQ ID NO: 14.

In an embodiment, the present disclosure provides an isolated human antibody or antibody fragment specific for human CD3 epsilon, wherein said antibody or antibody fragment comprises a variable heavy chain (VH) comprising the amino acid sequence of SEQ ID NO: 15.

In an embodiment, the present disclosure provides an isolated human antibody or antibody fragment specific for human CD3 epsilon, wherein said antibody or antibody fragment comprises a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure provides an isolated human antibody or antibody fragment specific for human CD3 epsilon, wherein said antibody or antibody fragment comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure provides an isolated human antibody or antibody fragment specific for human CD3 epsilon, wherein said antibody or antibody fragment comprises
a) a variable heavy chain (VH) comprising the amino acid sequence of SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure provides an isolated human antibody or antibody fragment specific for human CD3 epsilon, wherein said antibody or antibody fragment comprises
a) a variable heavy chain (VH) comprising the amino acid sequence of SEQ ID NO: 14 or SEQ ID NO: 15; and
b) a variable light chain (VL) comprising an amino acid sequence selected from the group consisting of SEQ ID NO: 17, SEQ ID NO: 18, SEQ ID NO: 24, SEQ ID NO: 25, and SEQ ID NO: 29.

In an embodiment, the present disclosure provides an isolated human antibody or antibody fragment specific for human CD3 epsilon, wherein said antibody or antibody fragment comprises a variable heavy chain (VH) and a variable light chain (VH) selected from the group consisting of:
a) the VH comprising the amino acid sequence of SEQ ID NO: 13 and the VL comprising the amino acid sequence of SEQ ID NO: 16,
b) the VH comprising the amino acid sequence of SEQ ID NO: 14 and the VL comprising the amino acid sequence of SEQ ID NO: 16,
c) the VH comprising the amino acid sequence of SEQ ID NO: 13 and the VL comprising the amino acid sequence of SEQ ID NO: 17,
d) the VH comprising the amino acid sequence of SEQ ID NO: 14 and the VL comprising the amino acid sequence of SEQ ID NO: 17,
e) the VH comprising the amino acid sequence of SEQ ID NO: 14 and the VL comprising the amino acid sequence of SEQ ID NO: 18,
f) the VH comprising the amino acid sequence of SEQ ID NO: 14 and the VL comprising the amino acid sequence of SEQ ID NO: 19,
g) the VH comprising the amino acid sequence of SEQ ID NO: 14 and the VL comprising the amino acid sequence of SEQ ID NO: 20,
h) the VH comprising the amino acid sequence of SEQ ID NO: 14 and the VL comprising the amino acid sequence of SEQ ID NO: 21,
i) the VH comprising the amino acid sequence of SEQ ID NO: 14 and the VL comprising the amino acid sequence of SEQ ID NO: 22,
j) the VH comprising the amino acid sequence of SEQ ID NO: 14 and the VL comprising the amino acid sequence of SEQ ID NO: 23,
k) the VH comprising the amino acid sequence of SEQ ID NO: 14 and the VL comprising the amino acid sequence of SEQ ID NO: 24,
l) the VH comprising the amino acid sequence of SEQ ID NO: 14 and the VL comprising the amino acid sequence of SEQ ID NO: 25,
m) the VH comprising the amino acid sequence of SEQ ID NO: 14 and the VL comprising the amino acid sequence of SEQ ID NO: 26,
n) the VH comprising the amino acid sequence of SEQ ID NO: 14 and the VL comprising the amino acid sequence of SEQ ID NO: 27,
o) the VH comprising the amino acid sequence of SEQ ID NO: 14 and the VL comprising the amino acid sequence of SEQ ID NO: 28,
p) the VH comprising the amino acid sequence of SEQ ID NO: 14 and the VL comprising the amino acid sequence of SEQ ID NO: 29,
q) the VH comprising the amino acid sequence of SEQ ID NO: 14 and the VL comprising the amino acid sequence of SEQ ID NO: 30,
r) the VH comprising the amino acid sequence of SEQ ID NO: 14 and the VL comprising the amino acid sequence of SEQ ID NO: 31,
s) the VH comprising the amino acid sequence of SEQ ID NO: 14 and the VL comprising the amino acid sequence of SEQ ID NO: 32,
t) the VH comprising the amino acid sequence of SEQ ID NO: 14 and the VL comprising the amino acid sequence of SEQ ID NO: 33,
u) the VH comprising the amino acid sequence of SEQ ID NO: 14 and the VL comprising the amino acid sequence of SEQ ID NO: 34,
v) the VH comprising the amino acid sequence of SEQ ID NO: 14 and the VL comprising the amino acid sequence of SEQ ID NO: 35,
w) the VH comprising the amino acid sequence of SEQ ID NO: 14 and the VL comprising the amino acid sequence of SEQ ID NO: 36,
x) the VH comprising the amino acid sequence of SEQ ID NO: 14 and the VL comprising the amino acid sequence of SEQ ID NO: 37,
y) the VH comprising the amino acid sequence of SEQ ID NO: 14 and the VL comprising the amino acid sequence of SEQ ID NO: 38,
z) the VH comprising the amino acid sequence of SEQ ID NO: 15 and the VL comprising the amino acid sequence of SEQ ID NO: 17,
aa) the VH comprising the amino acid sequence of SEQ ID NO: 15 and the VL comprising the amino acid sequence of SEQ ID NO: 18,
bb) the VH comprising the amino acid sequence of SEQ ID NO: 15 and the VL comprising the amino acid sequence of SEQ ID NO: 19,
cc) the VH comprising the amino acid sequence of SEQ ID NO: 15 and the VL comprising the amino acid sequence of SEQ ID NO: 20,
dd) the VH comprising the amino acid sequence of SEQ ID NO: 15 and the VL comprising the amino acid sequence of SEQ ID NO: 21,
ee) the VH comprising the amino acid sequence of SEQ ID NO: 15 and the VL comprising the amino acid sequence of SEQ ID NO: 22,
ff) the VH comprising the amino acid sequence of SEQ ID NO: 15 and the VL comprising the amino acid sequence of SEQ ID NO: 23,
gg) the VH comprising the amino acid sequence of SEQ ID NO: 15 and the VL comprising the amino acid sequence of SEQ ID NO: 24,
hh) the VH comprising the amino acid sequence of SEQ ID NO: 15 and the VL comprising the amino acid sequence of SEQ ID NO: 25,
ii) the VH comprising the amino acid sequence of SEQ ID NO: 15 and the VL comprising the amino acid sequence of SEQ ID NO: 26,
jj) the VH comprising the amino acid sequence of SEQ ID NO: 15 and the VL comprising the amino acid sequence of SEQ ID NO: 27,
kk) the VH comprising the amino acid sequence of SEQ ID NO: 15 and the VL comprising the amino acid sequence of SEQ ID NO: 28,
ll) the VH comprising the amino acid sequence of SEQ ID NO: 15 and the VL comprising the amino acid sequence of SEQ ID NO: 29,
mm) the VH comprising the amino acid sequence of SEQ ID NO: 15 and the VL comprising the amino acid sequence of SEQ ID NO: 30,
nn) the VH comprising the amino acid sequence of SEQ ID NO: 15 and the VL comprising the amino acid sequence of SEQ ID NO: 31,
oo) the VH comprising the amino acid sequence of SEQ ID NO: 15 and the VL comprising the amino acid sequence of SEQ ID NO: 32,
pp) the VH comprising the amino acid sequence of SEQ ID NO: 15 and the VL comprising the amino acid sequence of SEQ ID NO: 33,
qq) the VH comprising the amino acid sequence of SEQ ID NO: 15 and the VL comprising the amino acid sequence of SEQ ID NO: 34,
rr) the VH comprising the amino acid sequence of SEQ ID NO: 15 and the VL comprising the amino acid sequence of SEQ ID NO: 35,
ss) the VH comprising the amino acid sequence of SEQ ID NO: 15 and the VL comprising the amino acid sequence of SEQ ID NO: 36,
tt) the VH comprising the amino acid sequence of SEQ ID NO: 15 and the VL comprising the amino acid sequence of SEQ ID NO: 37, and
uu) the VH comprising the amino acid sequence of SEQ ID NO: 15 and the VL comprising the amino acid sequence of SEQ ID NO: 38.

In an embodiment, the present disclosure provides an isolated human antibody or antibody fragment specific for human CD3 epsilon, wherein said antibody or antibody fragment comprises a variable heavy chain (VH) and a variable light chain (VH) selected from the group consisting of:
a) the VH comprising the amino acid sequence of SEQ ID NO: 14 and the VL comprising the amino acid sequence of SEQ ID NO: 17,
b) the VH comprising the amino acid sequence of SEQ ID NO: 15 and the VL comprising the amino acid sequence of SEQ ID NO: 17,
c) the VH comprising the amino acid sequence of SEQ ID NO: 15 and the VL comprising the amino acid sequence of SEQ ID NO: 18,
d) the VH comprising the amino acid sequence of SEQ ID NO: 15 and the VL comprising the amino acid sequence of SEQ ID NO: 19,
e) the VH comprising the amino acid sequence of SEQ ID NO: 15 and the VL comprising the amino acid sequence of SEQ ID NO: 23,
f) the VH comprising the amino acid sequence of SEQ ID NO: 15 and the VL comprising the amino acid sequence of SEQ ID NO: 24,
g) the VH comprising the amino acid sequence of SEQ ID NO: 15 and the VL comprising the amino acid sequence of SEQ ID NO: 25,
h) the VH comprising the amino acid sequence of SEQ ID NO: 15 and the VL comprising the amino acid sequence of SEQ ID NO: 26,
i) the VH comprising the amino acid sequence of SEQ ID NO: 15 and the VL comprising the amino acid sequence of SEQ ID NO: 27,
j) the VH comprising the amino acid sequence of SEQ ID NO: 15 and the VL comprising the amino acid sequence of SEQ ID NO: 29,
k) the VH comprising the amino acid sequence of SEQ ID NO: 14 and the VL comprising the amino acid sequence of SEQ ID NO: 18,
l) the VH comprising the amino acid sequence of SEQ ID NO: 14 and the VL comprising the amino acid sequence of SEQ ID NO: 27, and
m) the VH comprising the amino acid sequence of SEQ ID NO: 14 and the VL comprising the amino acid sequence of SEQ ID NO: 29.

In an embodiment, the present disclosure provides an isolated human antibody or antibody fragment specific for human CD3 epsilon, wherein said antibody or antibody fragment comprises a variable heavy chain (VH) and a variable light chain (VH) selected from the group consisting of:
a) the VH comprising the amino acid sequence of SEQ ID NO: 14 and the VL comprising the amino acid sequence of SEQ ID NO: 17,
b) the VH comprising the amino acid sequence of SEQ ID NO: 15 and the VL comprising the amino acid sequence of SEQ ID NO: 17,
c) the VH comprising the amino acid sequence of SEQ ID NO: 15 and the VL comprising the amino acid sequence of SEQ ID NO: 18,
d) the VH comprising the amino acid sequence of SEQ ID NO: 15 and the VL comprising the amino acid sequence of SEQ ID NO: 24,
e) the VH comprising the amino acid sequence of SEQ ID NO: 15 and the VL comprising the amino acid sequence of SEQ ID NO: 25, abd
f) the VH comprising the amino acid sequence of SEQ ID NO: 14 and the VL comprising the amino acid sequence of SEQ ID NO: 29.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 13 and the variable light chain (VL) comprising the amino acid sequence of SEQ ID NO: 16.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 16.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 13 and the variable light chain (VL) comprising the amino acid sequence of SEQ ID NO: 17.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 17.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 18.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 19.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 20.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 21.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 22.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 23.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 24.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 25.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 26.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 27.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 28.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 29.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 30.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 31.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 32.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 33.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 34.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 35.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 36.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 37.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 14 and the variable light chain (VL) comprising the amino acid sequence of SEQ ID NO: 38.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 17.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 18.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 19.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 20.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 21.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 22.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 23.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 24.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 25.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 26.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 27.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 28.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 29.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 30.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 31.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 32.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 33.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 34.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 35.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 36.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 37.

In an embodiment, the present disclosure provides an isolated human antibody or antibody fragment specific for human CD3 epsilon, wherein said antibody or antibody fragment comprises the variable heavy chain (VH) comprising an amino acid sequence of SEQ ID NO: 15 and the variable light chain (VL) comprising the amino acid sequence of SEQ ID NO: 38.

Specificity

In an embodiment, the present disclosure provides an isolated human antibody or and antibody fragment specific for CD3 according to the present disclosure. In an embodiment, the said isolated human antibody or and antibody fragment specific for CD3 is specific for human CD3. In an embodiment, the said isolated human antibody or and antibody fragment specific for CD3 is specific for cynomolgus CD3. In an embodiment, the said isolated human antibody or antibody fragment specific for CD3 is specific for human and cynomolgus CD3.

In an embodiment, the isolated human antibody or and antibody fragment specific for CD3 according to the present disclosure is specific for human CD3 epsilon. In an embodiment, the isolated human antibody or and antibody fragment according to the present disclosure specific for CD3 according to the present disclosure is specific for cynomolgus CD3 epsilon. In an embodiment, the isolated human antibody or and antibody fragment specific for CD3 according to the present disclosure is specific for human and cynomolgus CD3 epsilon. In an embodiment, the isolated human antibody or and antibody fragment according to the present disclosure is specific for human and cynomolgus CD3 epsilon.

In an embodiment, the present disclosure provides an isolated human antibody or antibody fragment specific for human CD3, wherein said antibody or antibody fragment cross-reactively binds to cynomolgus CD3. In an embodiment, the present disclosure provides an isolated human antibody or and antibody fragment specific for human CD3 epsilon, wherein said antibody or antibody fragment cross-reactively binds to cynomolgus CD3 epsilon.

In an embodiment, the present disclosure provides an isolated human antibody or antibody fragment specific for CD3, wherein said antibody or antibody fragment specifically binds to human CD3 epsilon In an embodiment, said isolated human antibody or antibody fragment specific for CD3, specifically binds to human and cynomolgus CD3 epsilon.

In an embodiment, the present disclosure provides an isolated antibody or antibody fragment specific for human CD3 epsilon, wherein said human CD3 epsilon comprises the amino acid sequence SEQ ID NO: 1.

In an embodiment, the present disclosure provides an isolated human antibody or antibody fragment specific for cynomolgus CD3 epsilon, whereon said cynomolgus CD3 epsilon comprises the amino acid sequence SEQ ID NO: 2.

In an embodiment, the isolated human antibody or antibody fragment specific for CD3 epsilon according to the present disclosure specifically binds to a human CD3 epsilon polypeptide comprising the amino acid sequence of SEQ ID NO: 1. In an embodiment, the isolated human antibody or antibody fragment specific for CD3 epsilon according to the present disclosure specifically binds to a human CD3 epsilon polypeptide comprising the amino acid sequence of SEQ ID NO: 3. In an embodiment, the isolated human antibody or antibody fragment specific for CD3 epsilon according to the present disclosure specifically binds to a human CD3 epsilon polypeptide comprising the amino acid sequence of SEQ ID NO: 5. In an embodiment, the isolated human antibody or antibody fragment specific for CD3 epsilon according to the present disclosure specifically binds to a human CD3 epsilon polypeptide comprising the amino acid sequence of SEQ ID NO: 7 or SEQ ID NO: 8. In an embodiment, the isolated human antibody or antibody fragment specific for CD3 epsilon according to the present disclosure specifically binds to a human CD3 epsilon polypeptide comprising any of the amino acid sequence selected from the group consisting of SEQ ID NO: 1, 3, 5, 7, and 8.

In an embodiment, the isolated human antibody or antibody fragment specific for CD3 epsilon according to the present disclosure specifically binds to the extracellular region human CD3 epsilon. In an embodiment, the isolated human antibody or antibody fragment specific for CD3 epsilon according to the present disclosure specifically binds to the extracellular region human and cynomolgus CD3 epsilon.

In an embodiment, the isolated human antibody or antibody fragment specific for CD3 epsilon according to the present disclosure specifically binds to a cynomolgus CD3 epsilon polypeptide comprising the amino acid sequence of SEQ ID NO: 2. In an embodiment, the isolated human antibody or antibody fragment specific for CD3 epsilon according to the present disclosure specifically binds to a cynomolgus CD3 epsilon polypeptide comprising the amino acid sequence of SEQ ID NO: 4. In an embodiment, the isolated human antibody or antibody fragment specific for CD3 epsilon according to the present disclosure specifically binds to a cynomolgus CD3 epsilon polypeptide comprising the amino acid sequence of SEQ ID NO: 6. In an embodiment, the isolated human antibody or antibody fragment specific for CD3 epsilon according to the present disclosure specifically binds to a cynomolgus CD3 epsilon polypeptide comprising the amino acid sequence of SEQ ID NO: 104. In an embodiment, the isolated human antibody or antibody fragment specific for CD3 epsilon according to the present disclosure specifically binds to a cynomolgus CD3 epsilon polypeptide comprising any of the amino acid sequence selected from the group consisting of SEQ ID NO: 2, 4, 6, and 104.

In an embodiment, the present disclosure pertains to an isolated human antibody or antibody fragment specific for a polypeptide encoded by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 104 or SEQ ID NO: 8.

In an embodiment, the present disclosure pertains to an isolated human antibody or antibody fragment specific for a polypeptide encoded by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 104 and SEQ ID NO: 8.

In an embodiment, the present disclosure pertains to an isolated human antibody or antibody fragment according to the present disclosure which specifically binds to a polypeptide encoded by SEQ ID NO: 1, 3, 5, 7, or 8 and to a polypeptide encoded by SEQ ID NO: 2, 4, 6, or 104.

In an embodiment, the isolated human antibody or antibody or antibody fragment specific for human CD3 epsilon according to the present disclosure is cross-reactive to cynomolgus CD3 epsilon. In an embodiment, the isolated human antibody or antibody or antibody fragment specific for human CD3 epsilon according to the present disclosure cross-reactively binds to cynomolgus CD3 epsilon.

In an embodiment, the present disclosure pertains to an isolated human antibody or antibody fragment specific for human CD3 epsilon according to the present disclosure, wherein said antibody or antibody fragment has a monovalent affinity for a human CD3 epsilon peptide comprising SEQ ID NO: 7 or SEQ ID NO: 8 with a K_{D} of 10 nM or less, such as 8 nM or less, 7 nM or less, 6 nM or less, 5 nM or less, 4 nM or less, 3 nM or less, 2 nM or less, 1 nM or less, 0.1 nM or less, 0.2 nM, or 0.1 nM less.

In an embodiment, the present disclosure pertains to an isolated human antibody or antibody fragment specific for human CD3 epsilon according to the present disclosure, wherein said antibody or antibody fragment has a monovalent affinity for a human CD3 epsilon peptide comprising SEQ ID NO: 7 or SEQ ID NO: 8 with a K_{D} between 0.1 nM and 10 nM.

In an embodiment, the present disclosure pertains to an isolated human antibody or antibody fragment specific for human CD3 epsilon according to the present disclosure, wherein said antibody or antibody fragment has a monovalent affinity for a cynomolgus CD3 epsilon peptide comprising SEQ ID NO: 104 with a K_{D} of 10 nM or less, such as 8 nM or less, 7 nM or less, 6 nM or less, 5 nM or less, 4 nM or less, 3 nM or less, 2 nM or less, 1 nM or less, 0.1 nM or less, 0.2 nM, or 0.1 nM less.

In an embodiment, the present disclosure pertains to an isolated human antibody or antibody fragment specific for human CD3 epsilon according to the present disclosure, wherein said antibody or antibody fragment has a monovalent affinity for a cynomolgus CD3 epsilon peptide comprising SEQ ID NO: 104 with a K_{D} between 0.1 nM and 10 nM.

In an embodiment, the present disclosure pertains to an isolated human antibody or antibody fragment specific for human CD3 epsilon according to the present disclosure, wherein said antibody or antibody fragment has a monovalent affinity for a human CD3 epsilon peptide comprising SEQ ID NO: 7 or SEQ ID NO: 8 and for a cynomolgus CD3 epsilon peptide comprising SEQ ID NO: 104 with a K_{D} between 0.1 nM and 10 nM.

In an embodiment, the present disclosure pertains to an isolated human antibody or antibody fragment specific for human CD3 epsilon according to the present disclosure, wherein said antibody or antibody fragment has a monovalent affinity for a human CD3 epsilon peptide comprising SEQ ID NO: 8 with a K_{D} between 0.1 nM and 5 nM and for a cynomolgus CD3 epsilon peptide comprising SEQ ID NO: 104 with a K_{D} between 0.1 nM and 1 nM.

In certain embodiments, said monovalent affinity is determined in an antibody Fv or Fab format. In an embodiment, said monovalent affinity is determined as described herein in Example 10 or Example 13.

In alternative embodiments, the present disclosure pertains to an isolated human antibody or antibody fragment specific for human CD3 epsilon according to the present disclosure, wherein said antibody or antibody fragment specifically binds to human and/or cynomolgus derived PBMCs with an EC₅₀ concentration between 1 nM and 10 nM as determined in a FACS assay as described herein in Example 14. In an embodiment, said EC₅₀ concentration is determined in the Fab format. In an embodiment, said antibody fragment is a Fab.

In an embodiment, said isolated human antibody or antibody fragment specific for human CD3 epsilon comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

Method of Treatment

In an embodiment, the present disclosure provides an isolated human antibody or antibody fragment specific for human CD3 epsilon according to the present disclosure for use as a medicament.

In an embodiment, the present disclosure provides an isolated human antibody or antibody fragment specific for human CD3 epsilon according to the present disclosure for use in stimulating T cell activation. In an embodiment, the present disclosure provides an isolated human antibody or antibody fragment specific for human CD3 epsilon according to the present disclosure for use in enhancing immune function in a subject having a cell proliferative disease. In an embodiment, the present disclosure provides a method of treating or delaying the progression of a cell proliferative disease in a subject in need thereof, the method comprising administering to said subject an effective amount of an isolated human antibody or antibody fragment specific for human CD3 epsilon according the present disclosure. In an embodiment, the isolated human antibody or antibody fragment specific for human CD3 epsilon according the present disclosure can be for use in treating or delaying progression of a cell proliferative disease in a subject in need thereof. In an embodiment, said cell proliferative disease is a cancer.

In an embodiment, the cancer is selected from the group consisting of esophageal cancer, stomach cancer, small intestine cancer, large intestine cancer, colorectal cancer, breast cancer, non-small cell lung cancer, non-Hodgkin's lymphoma (NHL), B cell lymphoma, B cell leukemia, multiple myeloma, renal cancer, prostate cancer, liver cancer, head and neck cancer, melanoma, ovarian cancer, mesothelioma, glioblastoma, germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

In an embodiment, the isolated human antibody or antibody fragment specific for human CD3 epsilon according to the present disclosure can be for use as a medicament. In an embodiment, the present disclosure refers to the isolated human antibody or antibody fragment specific for human CD3 epsilon according to the present disclosure for use in medicine. In an embodiment, the present disclosure refers to the isolated human antibody or antibody fragment specific for human CD3 epsilon according to the present disclosure for use in the preparation of a medicament. In an embodiment, the present disclosure provides the use of the isolated human antibody or antibody fragment specific for human CD3 epsilon according to the present disclosure for the manufacture of a medicament.

In an embodiment, the present disclosure provides the use of the isolated human antibody or antibody fragment specific for human CD3 epsilon according to the present disclosure for the manufacture of a medicament for treating or delaying progression of a cell proliferative disease. In an embodiment, the present disclosure provides the use of the isolated human antibody or antibody fragment specific for human CD3 epsilon according to the present disclosure for the manufacture of a medicament for enhancing immune function in a subject having a cell proliferative disease.

In an embodiment, the present disclosure provides a method of treating a subject in need thereof with the isolated human antibody or antibody fragment specific for human CD3 epsilon according to the present disclosure.

In embodiment, the isolated human antibody or antibody fragment specific for human CD3 epsilon according to the present disclosure or a pharmaceutical composition comprising said antibody or antibody fragment is administered subcutaneously, intravenously, intramuscularly, topically, orally, transdermally, intraperitoneally, intraorbitally, by implantation, by inhalation, intrathecally, intraventricularly, or intranasally. In an embodiment, the isolated human antibody or antibody fragment specific for human CD3 epsilon according to the present disclosure or pharmaceutical composition according to the present disclosure is administered intravenously.

In an embodiment, said isolated human antibody or antibody fragment specific for human CD3 epsilon comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

Safety

In certain embodiments, the present disclosure provides an isolated human antibody or antibody fragment specific for human CD3 epsilon according to the present disclosure that does not activate T cells. In certain embodiments, the present disclosure provides an isolated human antibody or antibody fragment specific for human CD3 epsilon according to the present disclosure that does not induce upregulation of CD69 expression on CD4 positive and/or CD8 positive T cells.

In an embodiment, said isolated human antibody or antibody fragment specific for human CD3 epsilon comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

Composition

In an embodiment, the present disclosure provides a pharmaceutical composition comprising the isolated human antibody or antibody fragment specific for human CD3 epsilon according to the present disclosure and a pharmaceutically acceptable carrier. Such carriers are well known in the art, and the skilled artisan will find a formulation and a route of administration best suited to treat a subject with the antibodies or antibody fragments according to the present disclosure.

In an embodiment, the present disclosure refers to the use of said pharmaceutical compositions comprising the isolated human antibody or antibody fragment specific for human CD3 epsilon according to the present disclosure in the preparation of a medicament for the treatment of a disease. In an embodiment the present disclosure refers to the use of said pharmaceutical composition for the treatment of a disease.

In an embodiment, the present disclosure provides a method of treating a cell proliferative disease in a subject, said method comprising administering a pharmaceutical composition comprising a therapeutically effective amount of the isolated human antibody or antibody fragment specific for human CD3 epsilon according to the present disclosure.

In an embodiment, the present disclosure provides a pharmaceutical composition comprising a combination of the isolated human antibody or antibody fragment specific for human CD3 epsilon according to the present disclosure and a second therapeutic agent. In an embodiment, said second therapeutic agent is any agent that is advantageously combined with said human antibody or antibody fragment specific for human CD3 epsilon according to the present disclosure.

The present disclosure provides therapeutic methods for stimulating T cell activation using the isolated human antibody or antibody fragment specific for human CD3 epsilon according to the present disclosure, wherein the therapeutic methods comprise administering a therapeutically effective amount of an pharmaceutical composition comprising said isolated human antibody or antibody fragment specific for CD3 epsilon according to the present disclosure to a subject in need thereof.

The present disclosure also provides therapeutic methods for redirecting T cell killing to a cancerous cell using the isolated human antibody or antibody fragment specific for human CD3 epsilon according to the present disclosure, wherein the therapeutic methods comprise administering a therapeutically effective amount of a pharmaceutical composition comprising said antibody or antibody fragment according to the present disclosure to a subject in need thereof.

In an embodiment, said isolated human antibody or antibody fragment specific for human CD3 epsilon comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

Multispecific Antibodies

The human CD3 epsilon specific human antibody or antibody fragment according to the present disclosure is preferentially to be used in multispecific antibody formats to target T cells expressing CD3 and to stimulate T cell activation, e.g., under circumstances where T cell mediated killing of specific cell types such as tumor cells is beneficial or desirable.

The isolated human antibody or antibody fragment specific for human CD3 epsilon according to the present disclosure can be linked to or co-expressed with another functional molecule, e.g., another peptide or protein. For example, an antibody or antibody fragment can be functionally linked (e.g., by chemical coupling, genetic fusion, noncovalent association or otherwise) to one or more other molecular entities, such as another antibody or antibody fragment to produce a bispecific or multispecific antibody with an e.g. second or third binding specificity. Bispecific or multispecific antibodies, capable of binding to two or more antigens are of great interest for therapeutic applications, as they allow for the simultaneous binding and inactivation of two or more target antigens, and as such represent an alternative approach to conventional combination therapies.

Accordingly, in certain embodiments, the present disclosure provides an isolated human antibody or antibody fragments specific for human CD3 epsilon, wherein said antibody or antibody fragment is a monospecific, bispecific or multispecific antibody or antibody fragment.

A multispecific antibody may contain antibody fragments being specific for different epitopes on the same target antigen or may contain antibody fragments specific for more than one target antigen. In the context of bispecific or multispecific antibodies according to the present disclosure, the cell surface target antigen can be a cancer-associated antigen (CAA). Non-limiting examples of cancer-associated antigen include, e.g., an antigen that is expressed on the surface of a tumor or cancerous cell.

Exemplary multispecific antibody formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab₂ bispecific formats (see, e.g., Klein et al. 2012, mAbs 4:6, 1-11). Preferably, the isolated human antibody or antibody fragment specific for human CD3 epsilon according to the present disclosure is used in a format as described in WO 2020/115115. Most preferably, the isolated human antibody or antibody fragment specific for human CD3 epsilon according to the present disclosure is used in the CyCAT format as described herein or in WO 2013/104804.

In an embodiment, the present disclosure provides a bispecific or multispecific antibody comprising a first antibody fragment specific for human CD3 epsilon and a second antibody fragment which binds a different antigen than said first antibody fragment, wherein said first antibody fragment specific for CD3 comprises any one of the antibodies or antibody fragments specific for human CD3 epsilon according the present disclosure. In an embodiment, said first antibody fragment specific for CD3 comprises any one of the antibodies or antibody fragments specific for human CD3 epsilon listed in Table 10.

In an embodiment, the present disclosure provides a multispecific antibody comprising a first antibody fragment specific for human CD3 epsilon and a second antibody fragment which binds a different antigen than said first antibody fragment, wherein said first antibody fragment specific for human CD3 epsilon comprises any one of the human antibodies or antibody fragments specific for human CD3 epsilon according the present disclosure. In an embodiment, said first antibody fragment specific for CD3 comprises any one of the antibodies or antibody fragments specific for human CD3 epsilon listed in Table 10.

In an embodiment, said second antibody fragment specifically binds to a cell surface antigen. In an embodiment, said first antibody fragment binds to CD3 present on an immune effector cell. In an embodiment, the cell surface target antigen is a cancer-associated antigen. In an embodiment, said immune effector cell is a T cell. In an embodiment, said T cell is a cytotoxic T lymphocytes.

In an embodiment, the present disclosure provides a multispecific antibody comprising the human antibody or antibody fragment specific for human CD3 epsilon according to the present disclosure, wherein the multispecific antibody mediates redirected T cell killing of target antigen expressing cells. In certain embodiments, said target cell killing can be determined by the method as described herein in Example 11. In an embodiments, the multispecific antibody according to the present disclosure specifically binds a) CD3 expressed on a T cell and b) a second antigen present on a target cell other than the T-cell. In certain embodiments, said multispecific antibody activates the T cell following binding to (a) and (b). In some embodiments, the activated T cell is capable of exerting a cytotoxic effect and/or an apoptotic effect on a target cell.

In an embodiment, the present disclosure provides a multispecific antibody as disclosed herein, comprising the human antibody or antibody fragment specific for human CD3 epsilon according to the present disclosure, wherein said multispecific antibody does not induce upregulation of CD69 expression on CD4 positive and CD8 positive T cells in the absence of a cell surface antigen expressing cell. In embodiments, the present disclosure provides a multispecific antibody comprising the human antibody or antibody fragment specific for human CD3 epsilon according to the present disclosure, wherein said multispecific antibody induces human T cell proliferation in the presence of a cell surface target antigen expressing cell. In embodiments, the present disclosure provides a multispecific antibody as disclosed herein comprising the human antibody or antibody fragment specific for human CD3 epsilon according to the present disclosure, wherein said multispecific antibody does not activate T cells in the absence of cell surface antigen expressing cells.

In an embodiment, said human antibody or antibody fragment specific for human CD3 epsilon comprises
a) a variable heavy chain (VH) comprising the amino acid sequence of SEQ ID NO: 13, SEQ ID NO: 14 or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

CyCAT®

The isolated human antibody or antibody fragment specific for human CD3 epsilon according to the present disclosure may be used in the CyCAT approach as described herein or as described in WO 2013/104804 (which is incorporated herein in its entirety).

As parts of the aforementioned approach, two polypeptides are designed, each composed of an antibody fragment (e.g. a single-chain variable fragment (scFv) or an antibody Fab fragment) fused to either the variable light chain domain (VL) or the complementary variable heavy chain (VH) domain of a T cell activating anti-CD3 antibody. The unpaired VH or VL domain alone are not able to bind to CD3 and thus are not functional. However, once the two CyCAT polypeptides bind to their respective antigen on the surface of a cell, the complementary VL and VH domains come in close proximity and interact with each other to reconstitute the original CD3 antibody fragment (e.g. Fv domain) and become functional. The thus on-target cell formed trispecific heterodimer engages and stimulates T-cells for tumor cell destruction such as a conventional multispecific antibodies.

Accordingly, the present disclosure provides the CyCAT format comprising the human antibody or antibody fragment specific for human CD3 epsilon according to the present disclosure.

Thus, in an embodiment, the present disclosure provides a set of polypeptides comprising:
a) a first polypeptide comprising
   (i) a first targeting moiety, wherein said first targeting moiety specifically binds to a first antigen, and
   (ii) a first fragment of a functional domain,
   wherein neither said first fragment of the functional domain by itself nor said first polypeptide by itself is functional with respect to the function of said functional domain, and
b) a second polypeptide comprising
   (i) a second targeting moiety,
   wherein said second targeting moiety specifically binds to a second antigen, and
   (ii) a second fragment of said functional domain,
   wherein neither said second fragment of the functional domain by itself nor said second polypeptide by itself is functional with respect to the function of said functional domain,
   wherein said first polypeptide and said second polypeptide are not associated with each in the absence of a cell that has both, said first and second antigen at its cell surface,
   wherein, upon dimerization of said first fragment of said first polypeptide with said second fragment of said second polypeptide, the resulting dimer is functional with respect to the function of said functional domain,
   and wherein said first fragment of said functional domain comprises the VL of the human antibody or antibody fragment specific for human CD3 epsilon according to the present disclosure and said second fragment of said functional domain comprises the VH of the same human antibody or antibody fragment specific for human CD3 epsilon according to the present disclosure;
   or wherein said first fragment of said functional domain comprises the VH of the human antibody or antibody fragment specific for human CD3 epsilon according to the present disclosure and said second fragment of said functional domain comprises the VL of the same human antibody or antibody fragment specific for human CD3 epsilon according to the present disclosure,
   and wherein said functional domain is or comprises a Fv domain.

In an embodiment, said functional domain is a Fv domain.

In an embodiment, the cell carrying both, the first and second antigen at its cell surface induces dimerization of the first fragment of said functional domain of said first polypeptide with the second fragment of said functional domain of said second polypeptide, whereas a cell which does not carry both, the first and second antigen at its cell surface does not induce dimerization of the first fragment of said functional domain of said first polypeptide with the second fragment of said functional domain of said second polypeptide.

In an alternative embodiment, the present disclosure provides a set of polypeptides comprising:
a) a first polypeptide comprising
   (i) a first targeting moiety, wherein said first targeting moiety specifically binds to a first antigen, and
   (ii) a VH or VL of a Fv domain,
   wherein neither said VH or VL of said Fv domain by itself nor said first polypeptide by itself is functional with respect to the function of said Fv domain, and
b) a second polypeptide comprising
   (i) a second targeting moiety, wherein said second targeting moiety specifically binds to a second antigen, and
   (ii) the complementary VH or VL of said Fv domain,
   wherein neither said complementary VH or VL of said Fv domain by itself nor said second polypeptide by itself is functional with respect to the function of said Fv domain,
   wherein said first polypeptide and said second polypeptide are not associated with each in the absence of a cell that has said first antigen and said second antigen at its cell surface,
   wherein, upon dimerization of said VH of said first polypeptide with said VL of said second polypeptide, the resulting dimer is functional with respect to the function of said Fv domain, or
   wherein, upon dimerization of said VL of said first polypeptide with said VH of said second polypeptide, the resulting dimer is functional with respect to the function of said Fv domain,
   and wherein said VL comprises a VL of an human antibody or antibody fragment specific for human CD3 epsilon according to the present disclosure and said VH domain comprises the VH domain of the same human antibody or antibody fragment specific for human CD3 epsilon according to the present disclosure.

In an embodiment, the cell carrying both, the first and second antigen at its cell surface induces dimerization of the VH of said first polypeptide with the VL of said second polypeptide, whereas a cell which does not carry both, the first and second antigen at its cell surface does not induce dimerization of the VH of said first polypeptide with the VL of said second polypeptide or

wherein, the cell carrying both, the first and second antigen at its cell surface induces dimerization of the VL of said first polypeptide with the VH of said second polypeptide, whereas a cell which does not carry both, the first and second antigen at its cell surface does not induce dimerization of the VL of said first polypeptide with the VH of said second polypeptide.

In an embodiment, said Fv comprises an VH and VL selected from the group consisting of:
a) the VH comprising an amino acid sequence of SEQ ID NO: 13 and the VL comprising an amino acid sequence of SEQ ID NO: 16,
b) the VH comprising an amino acid sequence of SEQ ID NO: 14 and the VL comprising an amino acid sequence of SEQ ID NO: 16,
c) the VH comprising an amino acid sequence of SEQ ID NO: 13 and the VL comprising an amino acid sequence of SEQ ID NO: 17,
d) the VH comprising an amino acid sequence of SEQ ID NO: 14 and the VL comprising an amino acid sequence of SEQ ID NO: 17,
e) the VH comprising an amino acid sequence of SEQ ID NO: 14 and the VL comprising an amino acid sequence of SEQ ID NO: 18,
f) the VH comprising an amino acid sequence of SEQ ID NO: 14 and the VL comprising an amino acid sequence of SEQ ID NO: 19,
g) the VH comprising an amino acid sequence of SEQ ID NO: 14 and the VL comprising an amino acid sequence of SEQ ID NO: 20,
h) the VH comprising an amino acid sequence of SEQ ID NO: 14 and the VL comprising an amino acid sequence of SEQ ID NO: 21,
i) the VH comprising an amino acid sequence of SEQ ID NO: 14 and the VL comprising an amino acid sequence of SEQ ID NO: 22,
j) the VH comprising an amino acid sequence of SEQ ID NO: 14 and the VL comprising an amino acid sequence of SEQ ID NO: 23,
k) the VH comprising an amino acid sequence of SEQ ID NO: 14 and the VL comprising an amino acid sequence of SEQ ID NO: 24,
l) the VH comprising an amino acid sequence of SEQ ID NO: 14 and the VL comprising an amino acid sequence of SEQ ID NO: 25,
m) the VH comprising an amino acid sequence of SEQ ID NO: 14 and the VL comprising an amino acid sequence of SEQ ID NO: 26,
n) the VH comprising an amino acid sequence of SEQ ID NO: 14 and the VL comprising an amino acid sequence of SEQ ID NO: 27,
o) the VH comprising an amino acid sequence of SEQ ID NO: 14 and the VL comprising an amino acid sequence of SEQ ID NO: 28,
p) the VH comprising an amino acid sequence of SEQ ID NO: 14 and the VL comprising an amino acid sequence of SEQ ID NO: 29,
q) the VH comprising an amino acid sequence of SEQ ID NO: 14 and the VL comprising an amino acid sequence of SEQ ID NO: 30,
r) the VH comprising an amino acid sequence of SEQ ID NO: 14 and the VL comprising an amino acid sequence of SEQ ID NO: 31,
s) the VH comprising an amino acid sequence of SEQ ID NO: 14 and the VL comprising an amino acid sequence of SEQ ID NO: 32,
t) the VH comprising an amino acid sequence of SEQ ID NO: 14 and the VL comprising an amino acid sequence of SEQ ID NO: 33,
u) the VH comprising an amino acid sequence of SEQ ID NO: 14 and the VL comprising an amino acid sequence of SEQ ID NO: 34,
v) the VH comprising an amino acid sequence of SEQ ID NO: 14 and the VL comprising an amino acid sequence of SEQ ID NO: 35,
w) the VH comprising an amino acid sequence of SEQ ID NO: 14 and the VL comprising an amino acid sequence of SEQ ID NO: 36,
x) the VH comprising an amino acid sequence of SEQ ID NO: 14 and the VL comprising an amino acid sequence of SEQ ID NO: 37,
y) the VH comprising an amino acid sequence of SEQ ID NO: 14 and the VL comprising an amino acid sequence of SEQ ID NO: 38,
z) the VH comprising an amino acid sequence of SEQ ID NO: 15 and the VL comprising an amino acid sequence of SEQ ID NO: 17,
aa) the VH comprising an amino acid sequence of SEQ ID NO: 15 and the VL comprising an amino acid sequence of SEQ ID NO: 18,
bb) the VH comprising an amino acid sequence of SEQ ID NO: 15 and the VL comprising an amino acid sequence of SEQ ID NO: 19,
cc) the VH comprising an amino acid sequence of SEQ ID NO: 15 and the VL comprising an amino acid sequence of SEQ ID NO: 20,
dd) the VH comprising an amino acid sequence of SEQ ID NO: 15 and the VL comprising an amino acid sequence of SEQ ID NO: 21,
ee) the VH comprising an amino acid sequence of SEQ ID NO: 15 and the VL comprising an amino acid sequence of SEQ ID NO: 22,
ff) the VH comprising an amino acid sequence of SEQ ID NO: 15 and the VL comprising an amino acid sequence of SEQ ID NO: 23,
gg) the VH comprising an amino acid sequence of SEQ ID NO: 15 and the VL comprising an amino acid sequence of SEQ ID NO: 24,
hh) the VH comprising an amino acid sequence of SEQ ID NO: 15 and the VL comprising an amino acid sequence of SEQ ID NO: 25,
ii) the VH comprising an amino acid sequence of SEQ ID NO: 15 and the VL comprising an amino acid sequence of SEQ ID NO: 26,
jj) the VH comprising an amino acid sequence of SEQ ID NO: 15 and the VL comprising an amino acid sequence of SEQ ID NO: 27,
kk) the VH comprising an amino acid sequence of SEQ ID NO: 15 and the VL comprising an amino acid sequence of SEQ ID NO: 28,
ll) the VH comprising an amino acid sequence of SEQ ID NO: 15 and the VL comprising an amino acid sequence of SEQ ID NO: 29,
mm) the VH comprising an amino acid sequence of SEQ ID NO: 15 and the VL comprising an amino acid sequence of SEQ ID NO: 30,
nn) the VH comprising an amino acid sequence of SEQ ID NO: 15 and the VL comprising an amino acid sequence of SEQ ID NO: 31,
oo) the VH comprising an amino acid sequence of SEQ ID NO: 15 and the VL comprising an amino acid sequence of SEQ ID NO: 32,
pp) the VH comprising an amino acid sequence of SEQ ID NO: 15 and the VL comprising an amino acid sequence of SEQ ID NO: 33,
qq) the VH comprising an amino acid sequence of SEQ ID NO: 15 and the VL comprising an amino acid sequence of SEQ ID NO: 34,
rr) the VH comprising an amino acid sequence of SEQ ID NO: 15 and the VL comprising an amino acid sequence of SEQ ID NO: 35,
ss) the VH comprising an amino acid sequence of SEQ ID NO: 15 and the VL comprising an amino acid sequence of SEQ ID NO: 36,
tt) the VH comprising an amino acid sequence of SEQ ID NO: 15 and the VL comprising an amino acid sequence of SEQ ID NO: 37, and
uu) the VH comprising an amino acid sequence of SEQ ID NO: 15 and the VL comprising an amino acid sequence of SEQ ID NO: 38.

In an embodiment, said VH is selected from the group consisting of SEQ ID NO 13, SEQ ID NO 14 and SEQ ID NO 15. In an embodiment, said VL is selected from the group consisting of SEQ ID NO 16 to SEQ ID NO: SEQ ID NO: 38. In an embodiment, said VH is selected from the group consisting of SEQ ID NO 13, SEQ ID NO 14 and SEQ ID NO 15 and said VL is selected from the group consisting of SEQ ID NO 16 to SEQ ID NO: 38.

In an embodiment, said VH is selected from the group consisting of SEQ ID NO: 14 or SEQ ID NO: 15 and said VL is selected from the group consisting of SEQ ID NO: 17, SEQ ID NO: 18, SEQ ID NO: 24, SEQ ID NO: 25, and SEQ ID NO: 29.

In an embodiment, said first antigen and/or said second antigen is an antigen expressed on the surface of cells of a tumor or on the surface of progenitor/precursor cells of a tumor. In an embodiment, said first antigen and/or said second antigen is an cancer associated antigen expressed on the surface of cells of a tumor or on the surface of progenitor/precursor cells of a tumor.

In an embodiment, said first antigen and said second antigen is the same antigen. In an embodiment, said first antigen and said antigen are different antigen. In an embodiment, the combination of the first antigen and the second antigen is only found on cancerous cells, and not on cells that are not cancerous. In an embodiment, the combination of the first antigen and the second antigen is specific for cancerous cells of a certain type of cancer.

In an embodiment, said first targeting moiety and/or said second targeting moiety comprises a scFv, a Fab, a F(ab′)₂ fragment of an antibody or a VHH antibody.

In an embodiment, the set of polypeptides according to the present disclosure are for use in the treatment of a patient who is suffering from a cancer or for use in diagnosis in a patient who is suffering from a cancer.

In an embodiment, the present disclosure provides a pharmaceutical composition comprising the set of polypeptides according to the present disclosure, said pharmaceutical composition further comprises a pharmaceutically acceptable carrier. In an embodiment, the present disclosure provides a two pharmaceutical composition each of them comprising one of the two polypeptides of the set of polypeptides according to the present disclosure, said pharmaceutical compositions further comprises a pharmaceutically acceptable carrier.

In an embodiment, the present disclosure a kit comprising the set of polypeptides according to the present disclosure. The set of polypeptides according to the present disclosure may be used for the prevention and treatment of diseases, which are mediated by biological pathways in which a target antigen of interest is involved.

In an embodiment, the present disclosure provides a method for inducing lysis of a cancer cell, comprising contacting said cancer target cell expressing said first and second antigen in the presence of a cytotoxic T cell with set of polypeptides according to the present disclosure.

In an embodiment, the present disclosure provides a method for inhibition of proliferation of a cancer cell comprising contacting said cancer cell expressing said first and second antigen in the presence of a cytotoxic T cell with an with set of polypeptides according to the present disclosure.

In an embodiment, the present disclosure provides a method for killing a cancer cell comprising contacting said cancer cell expressing said first and second antigen, in the presence of a cytotoxic T cell with a set of polypeptides according to the present disclosure.

In an embodiment, the present disclosure provides a method for inducing a cellular response in a cytotoxic T cell, comprising contacting said cytotoxic T cell in the presence of a cancer cell expressing said first and second antigen, with a set of polypeptides according to the present disclosure. In an embodiment, said cellular response is selected from the group consisting of: proliferation, differentiation, cytokine secretion, cytotoxic effector molecule release, cytotoxic activity, and expression of activation markers.

In an embodiment, the present disclosure provides a method for inducing T cell proliferation in the presence of a cancer cell, comprising contacting said cancer cell expressing said first and second antigen, in the presence of a T-cell with set of polypeptides according to the present disclosure. In an embodiment, the present disclosure provides a method for stimulating a primary T cell response in the presence of cancer cells, comprising contacting said cancer cells expressing said first and second antigen, in the presence of said T cell with set of polypeptides according to the present disclosure. In an embodiment, the present disclosure provides a method for re-directing cytotoxic activity of a T cell to a cancer cell, comprising contacting said cancer cell expressing said first and second antigen, in the presence of said T cell with a set of polypeptides according to the present disclosure

In an embodiment, the present disclosure provides the use of a set of polypeptides according to the present disclosure for the treatment of cancer, comprising cancer cells expressing said first and second cancer associated antigen, in a subject, comprising:
(a) selecting a subject who is afflicted with a cancer,
(b) collecting one or more biological samples from the subject,
(c) identifying the said first and second cancer associated antigen expressing cancer cells in the one or more samples; and
(d) administering to the subject an effective amount of a set of polypeptides according to the present disclosure.

Further provided herein are combinations or sets of a first antigen binding molecule and a second antigen binding molecule, wherein each of the two antigen binding molecules is composed of a targeting moiety with specificity for a target antigen, fused to either an unpaired VL or VH domain of an antibody Fv domain specific for human CD3 epsilon and wherein the two antigen binding molecules are not associated by a covalent bond.

In an embodiment, the present disclosure pertains to an antigen binding molecule consisting from its N-terminus to its C-terminus of
a) a targeting moiety specific for a first antigen,
b) a peptide linker, and
c) either the VH domain of an antibody Fv domain specific for human CD3 epsilon selected from the group consisting of SEQ ID NO 13, SEQ ID NO 14 and SEQ ID NO 15 or the VL domain of the antibody Fv domain specific for human CD3 epsilon selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38,
   wherein the targeting moiety is fused to the N-terminus of either the VH or the VL domain of the antibody Fv domain specific for human CD3 epsilon via the peptide linker.

In an embodiment, the targeting moiety is fused via the peptide linker to the N-terminus of the VH domain of the antibody Fv domain specific for human CD3 epsilon. In an embodiment, the targeting moiety is fused via the peptide linker to the N-terminus of the VL domain of the antibody Fv domain specific for human CD3 epsilon.

In an embodiment, the targeting moiety is an antibody or antibody fragment. In an embodiment, the targeting moiety is selected from the group consisting of a Fab, scFab, Fab′, scFv, dsFv, and VHH. In an embodiment, the targeting moiety is a Fab.

In an embodiment of the present disclosure, the antigen binding molecule according to the present disclosure consists of two polypeptides, wherein
a) the first polypeptide consists of the light chain of a Fab, and
b) the second polypeptide consists from its N-terminus to its C-terminus
   I. the heavy chain of a Fab
   II. the peptide linker and
   III. either the VH domain of the antibody Fv domain specific for human CD3 epsilon selected from the group consisting of SEQ ID NO: 13, SEQ ID NO: 14 and SEQ ID NO 15 or the VL domain of the antibody Fv domain specific for human CD3 epsilon selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In an embodiment, the present disclosure pertains to a set of antigen binding molecules consisting of
a) a first antigen binding molecule consisting from its N-terminus to its C-terminus of
   i. a first targeting moiety specific for a first antigen,
   ii. a first peptide linker and
   iii. either the VH domain of an antibody Fv domain specific for human CD3 epsilon selected from the group consisting of SEQ ID NO: 13, SEQ ID NO: 14 and SEQ ID NO 15 or the VL domain of the antibody Fv domain specific for human CD3 epsilon, selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38,
   wherein the first targeting moiety is fused to the N-terminus of either the VH or VL domain of the antibody Fv domain specific for human CD3 epsilon of the first antigen binding molecule via the first peptide linker, and
b) a second antigen binding molecule consisting from its N-terminus to its C-terminus of
   I. a second targeting moiety specific for a second antigen,
   II. a second peptide linker, and
   III. either the VL domain of the antibody Fv domain specific for human CD3 epsilon selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38. if the first antigen binding molecule comprises the VH domain of an antibody Fv domain specific for human CD3 epsilon or the VH domain of the antibody Fv domain specific for human CD3 epsilon selected from the group consisting of SEQ ID NO: 13, SEQ ID NO: 14 and SEQ ID NO 15, if the first antigen binding molecule comprises the VL domain of the antibody Fv domain specific for human CD3 epsilon,
   wherein the second targeting moiety is fused to the N-terminus of either the VL or VH domain of the antibody Fv domain specific for human CD3 epsilon of the second antigen binding molecule via the second peptide linker.

In an embodiment, the first targeting moiety is fused via the first peptide linker to the N-terminus of the VH domain of the antibody Fv domain specific for human CD3 epsilon of the first antigen binding molecule. In an embodiment, the first targeting moiety is fused via the first peptide linker to the N-terminus of the VL domain of the antibody Fv domain specific for human CD3 epsilon of the first antigen binding molecule.

In an embodiment, the second targeting moiety is fused via the second peptide linker to the N-terminus of the VH domain of the antibody Fv domain specific for human CD3 epsilon of the second antigen binding molecule. In an embodiment, the second targeting moiety is fused via the second peptide linker to the N-terminus of the VL domain of the antibody Fv domain specific for human CD3 epsilon of the second antigen binding molecule.

In an embodiment, the targeting moiety is an antibody or antibody fragment. In an embodiment, the targeting moiety is selected from the group consisting of Fab, scFab, Fab′, scFv, dsFv, and VHH. In an embodiment, the targeting moiety is a Fab.

In an embodiment, the peptide linker used in an antigen binding molecules according to the present disclosure has a length of 5 to 20 amino acids residues. In an embodiment, the peptide linker used in antigen binding molecules according to the present disclosure has a length of 5 amino acids residues. In an embodiment, the peptide linker comprises the amino acid sequence of: GQPSG (SEQ ID NO: 105). In an embodiment, the peptide linker consists of the amino acid sequence of: GQPSG (SEQ ID NO: 105).

In an embodiment of the present disclosure, in the set of antigen binding molecules according to the present disclosure, the first antigen binding molecule and the second antigen binding molecule are not linked by a covalent bond. In an embodiment, the first antigen binding molecule and the second antigen binding molecule according to the present disclosure are capable of forming a heteromeric molecule. In an embodiment, the amount of the heteromeric molecule formed under conditions in which cells expressing the first antigen and the second antigen are present is higher than that under conditions in which the cells are not present or in which cells expressing either the first or the second antigen are present.

In an embodiment of the present disclosure, the VH and VL domain of the antibody Fv domain specific for human CD3 epsilon are capable of non-covalently associating thereby forming the antibody Fv domain specific for human CD3 epsilon. In an embodiment, the non-covalent association of said VH and VL domain specific human CD3 epsilon results in the formation of the antibody Fv domain specific for human CD3 epsilon. In an embodiment, the non-covalent association of the VH and VL domain specific human CD3 epsilon dimerizes the first and second antigen binding molecule. In an embodiment, said non-covalently association dimerizes the first and second antigen binding molecule resulting in the formation of a trispecific heteromeric antibody. In an embodiment, said formation of a trispecific heteromeric antibody occurs on-cell. In an embodiment, said formation of a trispecific heteromeric antibody occurs in vivo. In an embodiment, said formation of a trispecific heteromeric antibody molecule occurs in vitro. In an embodiment of the present disclosure, the either VH or VL domain of the antibody Fv domain specific for human CD3 epsilon of the first antigen binding molecule and the complementary VH or VL domain specific for human CD3 epsilon of the second antigen binding molecule are capable of non-covalently associating thereby forming an antibody Fv domain specific for human CD3 epsilon. In an embodiment of the present disclosure, the non-covalent association of the VH and VL domain specific human CD3 epsilon preferentially occurs when cells expressing the first antigen and the second antigen are present. In an embodiment, said non-covalent association of the VH and VL domain specific human CD3 epsilon preferentially occurs when cells expressing the first antigen and the second antigen are present and when both antigen binding molecules are bound to their antigen on the cell.

In an embodiment, said on-cell formed trispecific heterodimeric antibody has monovalent binding for the first antigen, monovalent binding to the second antigen and monovalent binding to human CD3 epsilon. In an embodiment of the present disclosure, the on-cell formed trispecific heterodimeric antibody is capable of engaging and stimulating cytotoxic T-cells for destruction of the target cell.

In an embodiment of the present disclosure, the first antigen and the second antigen are present on the same cell. In an embodiment, said cell is a tumor cell. In an embodiment, the first antigen and the second antigen are tumor associated antigens.

A set of antigen binding molecules according to the present disclosure may be used for the prevention and treatment of diseases, which are mediated by biological pathways in which target antigens of interest are involved. This may be preferably achieved by recruiting cytotoxic immune cells, such as T-cells, to cells expressing the target antigens, preferably the CAAs. In an embodiment, the present disclosure provides a method for inducing lysis of a target cell, such as a tumor cell, comprising contacting said cell in the presence of a cytotoxic T-cell with a set of antigen binding molecule according to the present disclosure.

In an embodiment, the present disclosure provides a method for inhibition of signaling in a target cell, such as a tumor cell, comprising contacting said cell in the presence of a cytotoxic T-cell with a set of antigen binding molecule according to the present disclosure. In an embodiment, the present disclosure provides a method for inhibition of proliferation of a target cell, such as a tumor cell, comprising contacting said cell in the presence of a cytotoxic T-cell with a set of antigen binding molecules according to the present disclosure. In an embodiment, the present disclosure provides a method for inducing a cellular response in cytotoxic T-cells, comprising contacting said cytotoxic T-cell in the presence of a target cell, such as a tumor cell, with a set of antigen binding molecules according to the present disclosure. In an embodiment, said cellular response is selected from the group consisting of: proliferation, differentiation, cytokine secretion, cytotoxic effector molecule release, cytotoxic activity, and expression of activation markers.

In an embodiment, the present disclosure provides a method for inducing human T-cell proliferation in the presence of a target cell, such as a tumor cell, comprising contacting said cell in the presence of a T-cell with a set of antigen binding molecules according to the present disclosure. In an embodiment, the present disclosure provides a method for stimulating a primary T-cell response in the presence of a target cell, such as a tumor cell, comprising contacting said cell in the presence of said T-cell with a set of antigen binding molecules according to the present disclosure. In an embodiment, the present disclosure provides a method for re-directing cytotoxic activity of a T-cell to a target cell, such as a tumor cell, comprising contacting said cancer cells in the presence of said T-cell with a set of antigen binding molecule according to the present disclosure.

In an embodiment, the present disclosure provides the use of a set of antigen binding molecules according to the present disclosure for the treatment of cancer that is positive for at least two cancer associated antigen (CAA) in a subject, comprising:
(a) selecting a subject who is afflicted with a cancer,
(b) collecting one or more biological samples from the subject,
(c) identifying the at least two tumor associated antigens expressing cancer cells in the one or more samples, and
(d) administering to the subject an effective amount of a set of antigen binding molecule according to the present disclosure.

In an embodiment, said cancer cell expresses a first and second CAA. In a preferred embodiment, said first and second CAA are different. In an embodiment, in the set of antigen binding molecules according to the present disclosure, the first antigen binding molecule binds to a first CAA and the second antigen binding molecule binds to a second CAA.

Sequences

TABLE 2
Antibody variable domains and CDR sequences
of the parental CD3 specific antibody
MAB-1 (CDRs are provided in combined
Kabat + Chothia annotation)

| | VH/ | SEQ | |
|---|---|---|---|
| | VL/ | ID | |
| Antibody | CDR | NO: | [aa] |
| MAB-1 | VH | 13 | EVQLVESGGGLVQPGGSLRLSCA |
| | | | ASGFTFSSYWMSWVRQAPGKGLE |
| | | | WVANIKQDGSEKYYVDSVKGRFT |
| | | | ISRDNAKNSLYLQMNSLRAEDTA |
| | | | VYYCARGYSAEFAHRSGLDVWGQ |
| | | | GTLVTVSS |
| (parental) | VL | 16 | QSVLTQPPSASGTPGQRVTISCS |
| | | | GSSSNIGSNYVYWYQQLPGTAPK |
| | | | LLIYRNNQRPSGVPDRFSGSKSG |
| | | | TSASLAISGLRSEDEADYYCAAW |
| | | | DHHRSGAVFGGGTKLTVLGQ |
| | HCDR1 | 40 | GFTFSSYWMS |
| | HCDR2 | 43 | NIKQDGSEKYYVDSVKG |
| | HCDR3 | 39 | GYSAEFAHRSGLDV |
| | LCDR1 | 46 | SGSSSNIGSNYVY |
| | LCDR2 | 51 | RNNQRPS |
| | LCDR3 | 60 | AAWDHHRSGAV |

TABLE 3
Affinity matured variants with improved
variable domains of MAB-1 (CDRs are
provided in combined Kabat + Chothia
annotation)

| | VH/ | SEQ | |
|---|---|---|---|
| | VL/ | ID | |
| Antibody | CDR | NO: | [aa] |
| MAB-1_25 | VH | 14 | EVQLVESGGGLVQPGGSLRLSCAAS |
| | | | GFTFKSYYMSWVRQAPGKGLEWVAN |
| | | | IDYQSQHAYYAESVKGRFTISRDNA |
| | | | KNSLYLQMNSLRAEDTAVYYCARGY |
| | | | SAEFAHRSGLDVWGQGTLVTVSS |
| | VL | 16 | QSVLTQPPSASGTPGQRVTISCSGS |
| | | | SSNIGSNYVYWYQQLPGTAPKLLIY |
| | | | RNNQRPSGVPDRFSGSKSGTSASLA |
| | | | ISGLRSEDEADYYCAAWDHHRSGAV |
| | | | FGGGTKLTVLGQ |
| | HCDR1 | 41 | GFTFKSYYMS |
| | HCDR2 | 44 | NIDYQSQHAYYAESVKG |
| | HCDR3 | 39 | GYSAEFAHRSGLDV |
| | LCDR1 | 46 | SGSSSNIGSNYVY |
| | LCDR2 | 51 | RNNQRPS |
| | LCDR3 | 60 | AAWDHHRSGAV |
| MAB-1_2 | VH | 13 | EVQLVESGGGLVQPGGSLRLSCAASGF |
| | | | KTFSSYWMSWVRQAPGGLEWVANIKQD |
| | | | GSEKYYVDSVKGRFTISRDNAKNSLYL |
| | | | QMNSLRAEDTAVYYCARGYSAEFAHRS |
| | | | GLDVWGQGTLVTVSS |
| | VL | 17 | QSVLTQPPSASGTPGQRVTISCSGSSS |
| | | | NIGSNYVYWYQQLPGTAPKLLIYRNNQ |
| | | | RPSGVPDRFSGSKSGTSASLAISGLRS |
| | | | EDEADYYCAAWDRHSHGAVFGGGTKLT |
| | | | VLGQ |
| | HCDR1 | 40 | GFTFSSYWMS |
| | HCDR2 | 43 | NIKQDGSEKYYVDSVKG |
| | HCDR3 | 39 | GYSAEFAHRSGLDV |
| | LCDR1 | 46 | SGSSSNIGSNYVY |
| | LCDR2 | 51 | RNNQRPS |
| | LCDR3 | 61 | AAWDRHSHGAV |

TABLE 4
Improved VH domains and CDR sequences
of MAB-1 (CDRs are provided in
combined Kabat + Chothia annotation)

| | | SEQ | |
|---|---|---|---|
| | VH/ | ID | |
| Antibody | CDR | NO: | [aa] |
| MAB-1_ | VH | 13 | EVQLVESGGGLVQPGGSLRLS |
| 1_VH | | | CAASGFTFSSYWMSWVRQAPG |
| (parental) | | | KGLEWVANIKQDGSEKYYVDS |
| | | | VKGRFTISRDNAKNSLYLQMN |
| | | | SLRAEDTAVYYCARGYSAEFA |
| | | | HRSGLDVWGQGTLVTVSS |
| | HCDR1 | 40 | GFTFSSYWMS |
| | HCDR2 | 43 | NIKQDGSEKYYVDSVKG |
| | HCDR3 | 39 | GYSAEFAHRSGLDV |
| MAB-1_ | VH | 14 | EVQLVESGGGLVQPGGSLRLSC |
| 25_VH | | | AASGFTFKSYYMSWVRQAPGKG |
| | | | NLEWVANIDYQSQHAYYAESVKG |
| | | | RFTISRDNAKNSLYLQMSLRAED |
| | | | TAVYYCARGYSAEFAHRSGLDVW |
| | | | GQGTLVTVSS |
| | HCDR1 | 41 | GFTFKSYYMS |
| | HCDR2 | 44 | NIDYQSQHAYYAESVKG |
| | HCDR3 | 39 | GYSAEFAHRSGLDV |
| MAB-1_ | VH | 15 | EVQLVESGGGLVQPGGSLRLSCA |
| 24_VH | | | ASGFTFRSHYMTWVRQAPGKGLE |
| | | | WVANIDYEGTRTYYAESVKGRFT |
| | | | ISRDNAKNSLYLQMNSLRAEDTA |
| | | | VYYCARGYSAEFAHRSGLDVWGQ |
| | | | GTLVTVSS |
| | HCDR1 | 42 | GFTFRSHYMT |
| | HCDR2 | 45 | NIDYEGTRTYYAESVKG |
| | HCDR3 | 39 | GYSAEFAHRSGLDV |

TABLE 5
Improved VL domains and CDR sequences
of MAB-1 (CDRs are provided in
combined Kabat + Chothia annotation)

| | | SEQ | |
|---|---|---|---|
| | VL/ | ID | |
| Antibody | CDR | NO: | [aa] |
| MAB-1_ | VL | 16 | QSVLTQPPSASGTPGQRVTI |
| 1_VL | | | SCSGSSSNIGSNYVYWYQQL |
| parental | | | PGTAPKLLIYRNNQRPSGVP |
| | | | DRFSGSKSGTSASLAISGLR |
| | | | SEDEADYYCAAWDHHRSGAV |
| | | | FGGGTKLTVLGQ |
| | LCDR1 | 46 | SGSSSNIGSNYVY |
| | LCDR2 | 51 | RNNQRPS |
| | LCDR3 | 60 | AAWDHHRSGAV |
| MAB-1_ | VL | 17 | QSVLTQPPSASGTPGQRVTI |
| 2_VL | | | SCSGSSSNIGSNYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | LCDR1 | 46 | SGSSSNIGSNYVY |
| | LCDR2 | 51 | RNNQRPS |
| | LCDR3 | 61 | AAWDRHSHGAV |
| MAB-1_ | VL | 18 | QSVLTQPPSASGTPGQRVTI |
| 3_VL | | | SCSGSSSNIGINYVYWYQQL |
| | | | PGTAPKLLIYRNNIRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 52 | RNNIRPS |
| | LCDR3 | 61 | AAWDRHSHGAV |
| MAB-1_ | VL | 19 | QSVLTQPPSASGTPGQRVTI |
| 4_VL | | | SCSGSSSNIGINYVYWYQQL |
| | | | PGTAPKLLIYRNNKRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 53 | RNNKRPS |
| | LCDR3 | 61 | AAWDRHSHGAV |
| MAB-1_ | VL | 20 | QSVLTQPPSASGTPGQRVTI |
| 5_VL | | | SCSGSSSNIGANYVYWYQQL |
| | | | PGTAPKLLIYRNKKRPSGVP |
| | | | DRFSGSKSGTSASLAISGLR |
| | | | SEDEADYYCAAWDRHSHGAV |
| | | | FGGGTKLTVLGQ |
| | LCDR1 | 48 | SGSSSNIGANYVY |
| | LCDR2 | 54 | RNKKRPS |
| | LCDR3 | 61 | AAWDRHSHGAV |
| MAB-1_ | VL | 21 | QSVLTQPPSASGTPGQRVTI |
| 6_VL | | | SCSGSSSNIGINYVYWYQQL |
| | | | PGTAPKLLIYRNKQRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 55 | RNKQRPS |
| | LCDR3 | 61 | AAWDRHSHGAV |
| MAB-1_ | VL | 22 | QSVLTQPPSASGTPGQRVTI |
| 7_VL | | | SCSGSSSNIGQTYVYWYQQL |
| | | | PGTAPKLLIYRNSQRPSGVP |
| | | | DRFSGSKSGTSASLAISGLR |
| | | | SEDEADYYCAAWDRHSHGAV |
| | | | FGGGTKLTVLGQ |
| | LCDR1 | 49 | SGSSSNIGQTYVY |
| | LCDR2 | 56 | RNSQRPS |
| | LCDR3 | 61 | AAWDRHSHGAV |
| MAB-1_ | VL | 23 | QSVLTQPPSASGTPGQRVTI |
| 8_VL | | | SCSGSSSNIGINYVYWYQQL |
| | | | PGTAPKLLIYRNHIRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 57 | RNHIRPS |
| | LCDR3 | 61 | AAWDRHSHGAV |
| MAB-1_ | VL | 24 | QSVLTQPPSASGTPGQRVTI |
| 9_VL | | | SCSGSSSNIGANYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | LCDR1 | 48 | SGSSSNIGANYVY |
| | LCDR2 | 51 | RNNQRPS |
| | LCDR3 | 61 | AAWDRHSHGAV |
| MAB-1_ | VL | 25 | QSVLTQPPSASGTPGQRVTI |
| 10_VL | | | SCSGSSSNIGINYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 51 | RNNQRPS |
| | LCDR3 | 61 | AAWDRHSHGAV |
| MAB-1_ | VL | 26 | QSVLTQPPSASGTPGQRVTI |
| 11_VL | | | SCSGSSSNIGANYVYWYQQL |
| | | | PGTAPKLLIYRNKQRPSGVP |
| | | | DRFSGSKSGTSASLAISGLR |
| | | | SEDEADYYCAAWDRHSHGAV |
| | | | FGGGTKLTVLGQ |
| | LCDR1 | 48 | SGSSSNIGANYVY |
| | LCDR2 | 55 | RNKQRPS |
| | LCDR3 | 61 | AAWDRHSHGAV |
| MAB-1_ | VL | 27 | QSVLTQPPSASGTPGQRVTI |
| 12_VL | | | SCSGSSSNIGINYVYWYQQL |
| | | | PGTAPKLLIYRNTQRPSGVP |
| | | | DRFSGSKSGTSASLAISGLR |
| | | | SEDEADYYCAAWDRHSHGAV |
| | | | FGGGTKLTVLGQ |
| | LCDR1 | 47 | SGSSSNIGINYVY |
| | LCDR2 | 58 | RNTQRPS |
| | LCDR3 | 61 | AAWDRHSHGAV |
| MAB-1_ | VL | 28 | QSVLTQPPSASGTPGQRVTI |
| 13_VL | | | SCSGSSSNIGKNYVYWYQQL |
| | | | PGTAPKLLIYRNYQRPSGVP |
| | | | DRFSGSKSGTSASLAISGLR |
| | | | SEDEADYYCAAWDRHSHGAV |
| | | | FGGGTKLTVLGQ |
| | LCDR1 | 50 | SGSSSNIGKNYVY |
| | LCDR2 | 59 | RNYQRPS |
| | LCDR3 | 61 | AAWDRHSHGAV |
| MAB-1_ | VL | 29 | QSVLTQPPSASGTPGQRVTI |
| 14_VL | | | SCSGSSSNIGSNYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | LCDR1 | 46 | SGSSSNIGSNYVY |
| | LCDR2 | 51 | RNNQRPS |
| | LCDR3 | 62 | AGWDRRLHGAV |
| MAB-1_ | VL | 30 | QSVLTQPPSASGTPGQRVTI |
| 15_VL | | | SCSGSSSNIGQTYVYWYQQL |
| | | | PGTAPKLLIYRNTQRPSGVP |
| | | | DRFSGSKSGTSASLAISGLR |
| | | | SEDEADYYCAAWDRHSHGAV |
| | | | FGGGTKLTVLGQ |
| | LCDR1 | 49 | SGSSSNIGQTYVY |
| | LCDR2 | 58 | RNTQRPS |
| | LCDR3 | 61 | AAWDRHSHGAV |
| MAB-1_ | VL | 31 | QSVLTQPPSASGTPGQRVTI |
| 16_VL | | | SCSGSSSNIGINYVYWYQQL |
| | | | PGTAPKLLIYRNNKRPSGVP |
| | | | DRFSGSKSGTSASLAISGLR |
| | | | SEDEADYYCAGWDRRLHGAV |
| | | | FGGGTKLTVLGQ |
| | LCDR1 | 47 | SGSSSNIGINYVY |
| | LCDR2 | 53 | RNNKRPS |
| | LCDR3 | 62 | AGWDRRLHGAV |
| MAB-1_ | VL | 32 | QSVLTQPPSASGTPGQRVTI |
| 17_VL | | | SCSGSSSNIGINYVYWYQQL |
| | | | PGTAPKLLIYRNHIRPSGVP |
| | | | DRFSGSKSGTSASLAISGLR |
| | | | SEDEADYYCAGWDRRLHGAV |
| | | | FGGGTKLTVLGQ |
| | LCDR1 | 47 | SGSSSNIGINYVY |
| | LCDR2 | 57 | RNHIRPS |
| | LCDR3 | 62 | AGWDRRLHGAV |
| MAB-1_ | VL | 33 | QSVLTQPPSASGTPGQRVTI |
| 18_VL | | | SCSGSSSNIGANYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | LCDR1 | 48 | SGSSSNIGANYVY |
| | LCDR2 | 51 | RNNQRPS |
| | LCDR3 | 62 | AGWDRRLHGAV |
| MAB-1_ | VL | 34 | QSVLTQPPSASGTPGQRVTI |
| 19_VL | | | SCSGSSSNIGINYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | LCDR1 | 47 | SGSSSNIGINYVY |
| | LCDR2 | 51 | RNNQRPS |
| | LCDR3 | 62 | AGWDRRLHGAV |
| MAB-1_ | VL | 35 | QSVLTQPPSASGTPGQRVTI |
| 20_VL | | | SCSGSSSNIGANYVYWYQQL |
| | | | PGTAPKLLIYRNKQRPSGVP |
| | | | DRFSGSKSGTSASLAISGLR |
| | | | SEDEADYYCAGWDRRLHGAV |
| | | | FGGGTKLTVLGQ |
| | LCDR1 | 48 | SGSSSNIGANYVY |
| | LCDR2 | 55 | RNKQRPS |
| | LCDR3 | 62 | AGWDRRLHGAV |
| MAB-1_ | VL | 36 | QSVLTQPPSASGTPGQRVTI |
| 21_VL | | | SCSGSSSNIGINYVYWYQQL |
| | | | PGTAPKLLIYRNTQRPSGVP |
| | | | DRFSGSKSGTSASLAISGLR |
| | | | SEDEADYYCAGWDRRLHGAV |
| | | | FGGGTKLTVLGQ |
| | LCDR1 | 47 | SGSSSNIGINYVY |
| | LCDR2 | 58 | RNTQRPS |
| | LCDR3 | 62 | AGWDRRLHGAV |
| MAB-1_ | VL | 37 | QSVLTQPPSASGTPGQRVTI |
| 22_VL | | | SCSGSSSNIGQTYVYWYQQL |
| | | | PGTAPKLLIYRNTQRPSGVP |
| | | | DRFSGSKSGTSASLAISGLR |
| | | | SEDEADYYCAGWDRRLHGAV |
| | | | FGGGTKLTVLGQ |
| | LCDR1 | 49 | SGSSSNIGQTYVY |
| | LCDR2 | 58 | RNTQRPS |
| | LCDR3 | 62 | AGWDRRLHGAV |
| MAB-1_ | VL | 38 | QSVLTQPPSASGTPGQRVTI |
| 23_VL | | | SCSGSSSNIGINYVYWYQQL |
| | | | PGTAPKLLIYRNNIRPSGVP |
| | | | DRFSGSKSGTSASLAISGLR |
| | | | SEDEADYYCAGWDRRLHGAV |
| | | | FGGGTKLTVLGQ |
| | LCDR1 | 47 | SGSSSNIGINYVY |
| | LCDR2 | 52 | RNNIRPS |
| | LCDR3 | 62 | AGWDRRLHGAV |

TABLE 6
Sequences of CyCAT polypeptides carrying the improved
VL domains of MAB-1 (according Table 5)

| CyCAT polypeptide | SEQ | |
|---|---|---|
| [FabTrastuzumab-HC]- | ID | |
| anti-CD3-VL] | NO: | [aa] |
| CyCAT_MAB-1_1_VL | 63 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGSNYVYWYQQLPGTAPKLLIYRNNQRPSGVPDRFSGSK |
| | | SGTSASLAISGLRSEDEADYYCAAWDHHRSGAVFGGGTKLTV |
| | | LGQ |
| CyCAT_MAB-1_2_VL | 64 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGSNYVYWYQQLPGTAPKLLIYRNNQRPSGVPDRFSGSK |
| | | SGTSASLAISGLRSEDEADYYCAAWDRHSHGAVFGGGTKLTV |
| | | LGQ |
| CyCAT_MAB-1_3_VL | 65 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNIRPSGVPDRFSGSKS |
| | | GTSASLAISGLRSEDEADYYCAAWDRHSHGAVFGGGTKLTVL |
| | | GQ |
| CyCAT_MAB-1_4_VL | 66 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNKRPSGVPDRFSGSKS |
| | | GTSASLAISGLRSEDEADYYCAAWDRHSHGAVFGGGTKLTVL |
| | | GQ |
| CyCAT_MAB-1_5_VL | 67 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGANYVYWYQQLPGTAPKLLIYRNKKRPSGVPDRFSGSK |
| | | SGTSASLAISGLRSEDEADYYCAAWDRHSHGAVFGGGTKLTV |
| | | LGQ |
| CyCAT_MAB-1_6_VL | 68 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNKQRPSGVPDRFSGSKS |
| | | GTSASLAISGLRSEDEADYYCAAWDRHSHGAVFGGGTKLTVL |
| | | GQ |
| CyCAT_MAB-1_7_VL | 69 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGQTYVYWYQQLPGTAPKLLIYRNSQRPSGVPDRFSGSK |
| | | SGTSASLAISGLRSEDEADYYCAAWDRHSHGAVFGGGTKLTV |
| | | LGQ |
| CyCAT_MAB-1_8_VL | 70 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNHIRPSGVPDRFSGSKS |
| | | GTSASLAISGLRSEDEADYYCAAWDRHSHGAVFGGGTKLTVL |
| | | GQ |
| CyCAT_MAB-1_9_VL | 71 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGANYVYWYQQLPGTAPKLLIYRNNQRPSGVPDRFSGSK |
| | | SGTSASLAISGLRSEDEADYYCAAWDRHSHGAVFGGGTKLTV |
| | | LGQ |
| CyCAT_MAB-1_10_VL | 72 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNQRPSGVPDRFSGSKS |
| | | GTSASLAISGLRSEDEADYYCAAWDRHSHGAVFGGGTKLTVL |
| | | GQ |
| CyCAT_MAB-1_11_VL | 73 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGANYVYWYQQLPGTAPKLLIYRNKQRPSGVPDRFSGSK |
| | | SGTSASLAISGLRSEDEADYYCAAWDRHSHGAVFGGGTKLTV |
| | | LGQ |
| CyCAT_MAB-1_12_VL | 74 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNTQRPSGVPDRFSGSKS |
| | | GTSASLAISGLRSEDEADYYCAAWDRHSHGAVFGGGTKLTVL |
| | | GQ |
| CyCAT_MAB-1_13_VL | 75 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGKNYVYWYQQLPGTAPKLLIYRNYQRPSGVPDRFSGSK |
| | | SGTSASLAISGLRSEDEADYYCAAWDRHSHGAVFGGGTKLTV |
| | | LGQ |
| CyCAT_MAB-1_14_VL | 76 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGSNYVYWYQQLPGTAPKLLIYRNNQRPSGVPDRFSGSK |
| | | SGTSASLAISGLRSEDEADYYCAGWDRRLHGAVFGGGTKLTV |
| | | LGQ |
| CyCAT_MAB-1_15_VL | 77 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGQTYVYWYQQLPGTAPKLLIYRNTQRPSGVPDRFSGSK |
| | | SGTSASLAISGLRSEDEADYYCAAWDRHSHGAVFGGGTKLTV |
| | | LGQ |
| CyCAT_MAB-1_16_VL | 78 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNKRPSGVPDRFSGSKS |
| | | GTSASLAISGLRSEDEADYYCAGWDRRLHGAVFGGGTKLTVL |
| | | GQ |
| CyCAT_MAB-1_17_VL | 79 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNHIRPSGVPDRFSGSKS |
| | | GTSASLAISGLRSEDEADYYCAGWDRRLHGAVFGGGTKLTVL |
| | | GQ |
| CyCAT_MAB-1_18_VL | 80 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGANYVYWYQQLPGTAPKLLIYRNNQRPSGVPDRFSGSK |
| | | SGTSASLAISGLRSEDEADYYCAGWDRRLHGAVFGGGTKLTV |
| | | LGQ |
| CyCAT_MAB-1_19_VL | 81 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNQRPSGVPDRFSGSKS |
| | | GTSASLAISGLRSEDEADYYCAGWDRRLHGAVFGGGTKLTVL |
| | | GQ |
| CyCAT_MAB-1_20_VL | 82 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGANYVYWYQQLPGTAPKLLIYRNKQRPSGVPDRFSGSK |
| | | SGTSASLAISGLRSEDEADYYCAGWDRRLHGAVFGGGTKLTV |
| | | LGQ |
| CyCAT_MAB-1_21_VL | 83 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNTQRPSGVPDRFSGSKS |
| | | GTSASLAISGLRSEDEADYYCAGWDRRLHGAVFGGGTKLTVL |
| | | GQ |
| CyCAT_MAB-1_22_VL | 84 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGQTYVYWYQQLPGTAPKLLIYRNTQRPSGVPDRFSGSK |
| | | SGTSASLAISGLRSEDEADYYCAGWDRRLHGAVFGGGTKLTV |
| | | LGQ |
| CyCAT_MAB-1_23_VL | 85 | QVQLVESGGGLVQPGGSLRLSCAASGFNIKDTYIHWVRQAPG |
| | | KGLEWVARIYPTNGYTRYADSVKGRFTISADTSKNTAYLQMNS |
| | | LRAEDTAVYYCSRWGGDGFYAMDYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKKVEPKSCGQPSGQSVLTQPPSASGTPGQRVTISCSGS |
| | | SSNIGINYVYWYQQLPGTAPKLLIYRNNIRPSGVPDRFSGSKS |
| | | GTSASLAISGLRSEDEADYYCAGWDRRLHGAVFGGGTKLTVL |
| | | GQ |
| Fab-Light Chain (VL- | 86 | DIQMTQSPSSLSASVGDRVTITCRASQDVNTAVAWFQQKPGK |
| CL) of Trastuzumab | | APKLLIYSASFLYSGVPSRFSGSRSGTDFTLTISSLQPEDFATY |
| | | YCQQHYTTPPTFGQGTKVEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRGE |
| | | C |

TABLE 7
Sequences of CyCAT polypeptides carrying improved VH domains of MAB-1
(according Table 4)

| CyCAT polypeptide | SEQ | |
|---|---|---|
| [Fab-Trastuzumab- | ID | |
| HC]-anti-CD3-VL] | NO: | [aa] |
| CyCAT_MAB-1_1_VH | 87 | QVQLVESGGGLVQPGGSLRLSCAASGFNIKDTYIHWVRQ |
| | | APGKGLEWVARIYPTNGYTRYADSVKGRFTISADTSKNT |
| | | AYLQMNSLRAEDTAVYYCSRWGGDGFYAMDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCGQPSGEVQL |
| | | VESGGGLVQPGGSLRLSCAASGFTFSSYWMSWVRQAP |
| | | GKGLEWVANIKQDGSEKYYVDSVKGRFTISRDNAKNSLY |
| | | LQMNSLRAEDTAVYYCARGYSAEFAHRSGLDVWGQGTL |
| | | VTVSS |
| CyCAT_MAB-1_24_VH | 88 | QVQLVESGGGLVQPGGSLRLSCAASGFNIKDTYIHWVRQ |
| | | APGKGLEWVARIYPTNGYTRYADSVKGRFTISADTSKNT |
| | | AYLQMNSLRAEDTAVYYCSRWGGDGFYAMDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCGQPSGEVQL |
| | | VESGGGLVQPGGSLRLSCAASGFTFRSHYMTWVRQAPG |
| | | KGLEWVANIDYEGTRTYYAESVKGRFTISRDNAKNSLYLQ |
| | | MNSLRAEDTAVYYCARGYSAEFAHRSGLDVWGQGTLVT |
| | | VSS |
| CyCAT_MAB-1_25_VH | 89 | QVQLVESGGGLVQPGGSLRLSCAASGFNIKDTYIHWVRQ |
| | | APGKGLEWVARIYPTNGYTRYADSVKGRFTISADTSKNT |
| | | AYLQMNSLRAEDTAVYYCSRWGGDGFYAMDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCGQPSGEVQL |
| | | VESGGGLVQPGGSLRLSCAASGFTFKSYYMSWVRQAPG |
| | | KGLEWVANIDYQSQHAYYAESVKGRFTISRDNAKNSLYL |
| | | QMNSLRAEDTAVYYCARGYSAEFAHRSGLDVWGQGTLV |
| | | TVSS |
| Fab-Light Chain (VL- | 86 | DIQMTQSPSSLSASVGDRVTITCRASQDVNTAVAWFQQK |
| CL) of Trastuzumab | | PGKAPKLLIYSASFLYSGVPSRFSGSRSGTDFTLTISSLQP |
| | | EDFATYYCQQHYTTPPTFGQGTKVEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |

TABLE 8
Combinations of improved CD3 specific VH and VL domains incorporated into CyCAT
polypeptides (Fab light chain of Trastuzumab is not shown for CyCAT polypeptides)

| | Paired CyCAT polypeptides | | | | |
|---|---|---|---|---|---|
| | [Fab_Trastuzumab_HC]- | | | | |
| | Linker-[CD3-VL] × | | New anti-CD3 antibody | | |
| | [Fab_Trastuzumab_HC]- | SEQ ID | CD3 VL | SEQ ID | |
| CyCAT | Linker-[CD3-VH] | NO: | CD3 VH | NO: | anti-CD3 |
| CYCAT-7 | CyCAT_MAB-1_2_VL | 64 | MAB-1_2_VL | 17 | MAB-7 |
| | CyCAT_MAB-1_25_VH | 89 | MAB-1_25_VH | 14 | |
| CYCAT-8 | CyCAT_MAB-1_2_VL | 64 | MAB-1_2_VL | 17 | MAB-8 |
| | CyCAT_MAB-1_24_VH | 88 | MAB-1_24_VH | 15 | |
| CYCAT-9 | CyCAT_MAB-1_3_VL | 65 | MAB-1_3_VL | 18 | MAB-9 |
| | CyCAT_MAB-1_24_VH | 88 | MAB-1_24_VH | 15 | |
| CYCAT-10 | CyCAT_MAB-1_4_VL | 66 | MAB-1_4_VL | 19 | MAB-10 |
| | CyCAT_MAB-1_24_VH | 88 | MAB-1_24_VH | 15 | |
| CYCAT-11 | CyCAT_MAB-1_8_VL | 70 | MAB-1_8_VL | 23 | MAB-11 |
| | CyCAT_MAB-1_24_VH | 88 | MAB-1_24_VH | 15 | |
| CYCAT-12 | CyCAT_MAB-1_9_VL | 71 | MAB-1_9_VL | 24 | MAB-12 |
| | CyCAT_MAB-1_24_VH | 88 | MAB-1_24_VH | 15 | |
| CYCAT-13 | CyCAT_MAB-1_10_VL | 72 | MAB-1_10_VL | 25 | MAB-13 |
| | CyCAT_MAB-1_24_VH | 88 | MAB-1_24_VH | 15 | |
| CYCAT-14 | CyCAT_MAB-1_11_VL | 73 | MAB-1_11_VL | 26 | MAB-14 |
| | CyCAT_MAB-1_24_VH | 88 | MAB-1_24_VH | 15 | |
| CYCAT-15 | CyCAT_MAB-1_12_VL | 74 | MAB-1_12_VL | 27 | MAB-15 |
| | CyCAT_MAB-1_24_VH | 88 | MAB-1_24_VH | 15 | |
| CYCAT-16 | CyCAT_MAB-1_14_VL | 76 | MAB-1_14_VL | 29 | MAB-16 |
| | CyCAT_MAB-1_24_VH | 88 | MAB-1_24_VH | 15 | |
| CYCAT-17 | CyCAT_MAB-1_3_VL | 65 | MAB-1_3_VL | 18 | MAB-17 |
| | CyCAT_MAB-1_25_VH | 89 | MAB-1_25_VH | 14 | |
| CYCAT-18 | CyCAT_MAB-1_12_VL | 74 | MAB-1_12_VL | 27 | MAB-18 |
| | CyCAT_MAB-1_25_VH | 89 | MAB-1_25_VH | 14 | |
| CYCAT-19 | CyCAT_MAB-1_14_VL | 76 | MAB-1_14_VL | 29 | MAB-19 |
| | CyCAT_MAB-1_25_VH | 89 | MAB-1_25_VH | 14 | |

TABLE 9
Additional combinations of further improved CD3 specific VH
and VL domains domains incorporated into CyCAT polypeptides

| | Paired CyCAT polypeptides | | | | |
|---|---|---|---|---|---|
| | [Fab_Trastuzumab_HC]- | | | | |
| | Linker-[CD3-VL] × | | New anti-CD3 antibody | | |
| | [Fab_Trastuzumab_HC]- | SEQ ID | CD3_VL | SEQ ID | |
| CyCAT | Linker-[CD3-VH] | NO: | CD3_VH | NO: | anti-CD3 |
| CYCAT-20 | CyCAT_MAB-1_16_VL | 78 | MAB-1_16_VL | 31 | MAB-20 |
| | CyCAT_MAB-1_24_VH | 88 | MAB-1_24_VH | 15 | |
| CYCAT-21 | CyCAT_MAB-1_17_VL | 79 | MAB-1_17_VL | 32 | MAB-21 |
| | CyCAT_MAB-1_24_VH | 88 | MAB-1_24_VH | 15 | |
| CYCAT-22 | CyCAT_MAB-1_18_VL | 80 | MAB-1_18_VL | 33 | MAB-22 |
| | CyCAT_MAB-1_24_VH | 88 | MAB-1_24_VH | 15 | |
| CYCAT-23 | CyCAT_MAB-1_19_VL | 81 | MAB-1_19_VL | 34 | MAB-23 |
| | CyCAT_MAB-1_24_VH | 88 | MAB-1_24_VH | 15 | |
| CYCAT-24 | CyCAT_MAB-1_20_VL | 82 | MAB-1_20_VL | 35 | MAB-24 |
| | CyCAT_MAB-1_24_VH | 88 | MAB-1_24_VH | 15 | |
| CYCAT-25 | CyCAT_MAB-1_21_VL | 83 | MAB-1_21_VL | 36 | MAB-25 |
| | CyCAT_MAB-1_24_VH | 88 | MAB-1_24_VH | 15 | |
| CYCAT-26 | CyCAT_MAB-1_22_VL | 84 | MAB-1_22_VL | 37 | MAB-26 |
| | CyCAT_MAB-1_24_VH | 88 | MAB-1_24_VH | 15 | |
| CYCAT-27 | CyCAT_MAB-1_23_VL | 85 | MAB-1_23_VL | 38 | MAB-27 |
| | CyCAT_MAB-1_24_VH | 88 | MAB-1_24_VH | 15 | |

TABLE 10
New improved human CD3 specific human
antibodies according to the present
invention

| | VH/ | SEQ | |
|---|---|---|---|
| Anti-CD3 | VL | ID | |
| Antibody | domains | NO: | [aa] |
| MAB-7 | MAB-1_ | 17 | QSVLTQPPSASGTPGQRVTI |
| | 2_VL | | SCSGSSSNIGSNYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | MAB1_ | 14 | EVQLVESGGGLVQPGGSLRL |
| | 25_VH | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-8 | MAB-1_ | 17 | QSVLTQPPSASGTPGQRVTI |
| | 2_VL | | SCSGSSSNIGSNYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-9 | MAB-1_ | 18 | QSVLTQPPSASGTPGQRVTI |
| | 3_VL | | SCSGSSSNIGINYVYWYQQL |
| | | | PGTAPKLLIYRNNIRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-10 | MAB-1_ | 19 | QSVLTQPPSASGTPGQRVTI |
| | 4_VL | | SCSGSSSNIGINYVYWYQQL |
| | | | PGTAPKLLIYRNNKRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-11 | MAB-1_ | 23 | QSVLTQPPSASGTPGQRVTI |
| | 8_VL | | SCSGSSSNIGINYVYWYQQL |
| | | | PGTAPKLLIYRNHIRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-12 | MAB-1_ | 24 | QSVLTQPPSASGTPGQRVTI |
| | 9_VL | | SCSGSSSNIGANYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-13 | MAB-1_ | 25 | QSVLTQPPSASGTPGQRVTI |
| | 10_VL | | SCSGSSSNIGINYVYWYQQL |
| | | | PGTAPKLLIYRNNQRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-14 | MAB-1_ | 26 | QSVLTQPPSASGTPGQRVTI |
| | 11_VL | | SCSGSSSNIGANYVYWYQQL |
| | | | PGTAPKLLIYRNKQRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-15 | MAB-1_ | 27 | QSVLTQPPSASGTPGQRVTI |
| | 12_VL | | SCSGSSSNIGINYVYWYQQL |
| | | | PGTAPKLLIYRNTQRPSGVP |
| | | | DRFSGSKSGTSASLAISGLR |
| | | | SEDEADYYCAAWDRHSHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-16 | MAB-1_ | 29 | QSVLTQPPSASGTPGQRVTI |
| | 14_VL | | SCSGSSSNIGSNYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-17 | MAB-1_ | 18 | QSVLTQPPSASGTPGQRVTI |
| | 3_VL | | SCSGSSSNIGINYVYWYQQL |
| | | | PGTAPKLLIYRNNIRPSGVP |
| | | | DRFSGSKSGTSASLAISGLR |
| | | | SEDEADYYCAAWDRHSHGAV |
| | | | FGGGTKLTVLGQ |
| | MAB-1_ | 14 | EVQLVESGGGLVQPGGSLRL |
| | 25_VH | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-18 | MAB-1_ | 27 | QSVLTQPPSSGTPGQRVTIS |
| | 12_VL | | CSGSSSNIGINYVYWYQQLP |
| | | | GTAPKLLIYRNTQRPSGVPD |
| | | | RFSGSKSGTSASLAISGLRS |
| | | | EDEADYYCAAWDRHSHGAVF |
| | | | GGGTKLTVLGQ |
| | MAB-1_ | 14 | EVQLVESGGGLVQPGGSLRL |
| | 25_VH | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-19 | MAB-1_ | 29 | QSVLTQPPSASGTPGQRVTI |
| | 14_VL | | SCSGSSSNIGSNYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | MAB-1_ | 14 | EVQLVESGGGLVQPGGSLRL |
| | 25_VH | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-20 | MAB-1_ | 31 | QSVLTQPPSASGTPGQRVTI |
| | 16_VL | | SCSGSSSNIGINYVYWYQQL |
| | | | PGTAPKLLIYRNNKRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-21 | MAB-1_ | 32 | QSVLTQPPSASGTPGQRVTI |
| | 17_VL | | SCSGSSSNIGINYVYWYQQL |
| | | | PGTAPKLLIYRNHIRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-22 | MAB-1_ | 33 | QSVLTQPPSASGTPGQRVTI |
| | 18_VL | | SCSGSSSNIGANYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-23 | MAB-1_ | 34 | QSVLTQPPSASGTPGQRVTI |
| | 19_VL | | SCSGSSSNIGINYVYWYQQL |
| | | | PGTAPKLLIYRNNQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-24 | MAB-1_ | 35 | QSVLTQPPSASGTPGQRVTI |
| | 20_VL | | SCSGSSSNIGANYVYWYQQL |
| | | | PGTAPKLLIYRNKQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-25 | MAB-1_ | 36 | QSVLTQPPSASGTPGQRVTI |
| | 21_ | | SCSGSSSNIGINYVYWYQQL |
| | VL | | PGTAPKLLIYRNTQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-26 | MAB-1_ | 37 | QSVLTQPPSASGTPGQRVTI |
| | 22_VL | | SCSGSSSNIGQTYVYWYQQL |
| | | | PGTAPKLLIYRNTQRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |
| MAB-27 | MAB-1_ | 38 | QSVLTQPPSASGTPGQRVTI |
| | 23_VL | | SCSGSSSNIGINYVYWYQQL |
| | | | PGTAPKLLIYRNNIRPSGVP |
| | | | DRFSGSKSGTSASLAISGLR |
| | | | SEDEADYYCAGWDRRLHGAV |
| | | | FGGGTKLTVLGQ |
| | MAB-1_ | 15 | EVQLVESGGGLVQPGGSLRL |
| | 24_VH | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSS |

### WORKING EXAMPLES

### Example 1: Antigen Generation and Quality Control

Amino acid sequences of human CD3 epsilon and cynomolgus monkey CD3 epsilon were retrieved from publicly available sources (e.g. Uniprot), verified and produced in-house or by external service providers.

Antigens

As antigens for the initial pannings and screenings and later binding studies, linear peptides covering the N-terminal extracellular region of human and cynomolgus CD3 epsilon as well as fusion proteins of human CD3 epsilon and human CD3 delta were used. Peptides were either chemically synthesized or recombinantly produced. Chemically synthesized peptides were synthesized with a biotin tag, RP-HPLC purified and delivered as lyophilized material. The lyophilized peptides were stored at −80° C.

Recombinant Antigens

Cell Lines

The HEK293-6E cell line was developed by the National Research Council of Canada (NRC). HEK293-6E cells were maintained in Freestyle F17 medium (Thermo Scientific) in a humidified CO2-incubator at 37° C. and 6% CO2. HKB11 (Parental clone: U.S. Pat. No. 6,136,599. J. Biomed. Sci. 2002; 9:631-638) is a human hybrid cell line resulting from a fusion of HEK293 human embryonic kidney and 2B8 Burkitt lymphoma cells. HKB11 #52 cells were maintained in MAC1.0 medium containing 1% FCS in a humidified CO2 incubator at 37° C. and 6% CO2.

Recombinant Antigen Production

HKB11 #52 or HEK293-6E cells were transiently transfected one day post seeding with a commercially available transfection reagent according to the manufacturer's instructions. The cells were cultured for 3 days and the conditioned cell culture supernatant was harvested by centrifugation followed by sterile filtration (0.22 μm filter). Stably transfected HKB11 #52 pools were generated by transfection of cells followed by selection with 800 μg/mL G418 (Thermo Scientific). Expression of antigens from stable pools was done for 4 days post seeding.

CD3 FLAG-chLys-Avi-Tagged Antigens

DNA encoding the N-terminal extracellular region (ECD) of human or cynomolgus monkey CD3 epsilon or the fusion proteins of CD3 epsilon ECD with CD3 delta ECD including the natural leader sequence was cloned in frame with a C-terminal FLAG-chLys-Avi-tag into a pMAX expression vector, which is a modified expression vector based on pcDNA3.1 (Thermo Fisher). The antigens were purified by antibody affinity chromatography using self-made MOR003207 columns. After binding and washing the proteins were eluted with 0.1 M Glycine, pH 3.0. All affinity chromatography steps were performed using AKTA Express (GE Healthcare) chromatography systems. The samples were subsequently neutralized (with 3 M Tris pH 8), buffer-exchanged to PBS using PD10 columns (GE Healthcare) and sterile filtered (0.2 μm pore size).

CD3 Fc-Tagged Antigens

DNA encoding the N-terminal sequence of human or cynomolgus monkey CD3 epsilon including the natural leader sequence was cloned in frame with a C-terminal human IgG Fc-tag into a pMAX expression vector, which is a modified expression vector based on pcDNA3.1 (Thermo Fisher). The antigens were purified by Protein A chromatography using HiTrap MabSelect SuRe columns (GE Healthcare). After binding and washing the proteins were eluted with 100 mM Glycin pH 3.0. All affinity chromatography steps were performed using AKTA Express (GE Healthcare) chromatography systems. The samples were subsequently neutralized (with 3 M Tris pH 8), buffer-exchanged to PBS using PD10 columns (GE Healthcare) and sterile filtered (0.2 μm pore size). Biotinylation of CD3 antigens was performed by biotinylation using the BirA Kit (Avidity) followed by a preparative SEC using a Superdex 200 column (GE Healthcare). Protein concentrations were determined by UV-spectrophotometry. The quality of the samples was analyzed by denaturing, reducing or non-reducing SDS-PAGE, Streptavidin-Shift Assay, HP-SEC and DLS.

TABLE 11
Sequences of produced or synthesized CD3
antigens (without signal sequences).

| | SEQ | |
|---|---|---|
| | ID | |
| CD3 antigens | NO: | [aa] |
| Mature human CD3e | 3 | Pyr-DGNEEMGGITQ |
| (22-41)-bio | | TPYKVSIS-Biotin |
| (synthetic peptide) | | |
| Mature cynomolgus | 4 | Pyr-DGNEEMGSITQ |
| CD3e(22-41)-bio | | TPYQVSIS-Biotin |
| (synthetic peptide) | | |
| Mature human | 5 | QDGNEEMGGITQTPYK |
| CD3e(1-49)_ | | VSISGTTVILTCDIKT |
| Fc2(K105-K330) | | HTCPPCPAPELLGGPS |
| | | VFLFPPKPKDTLMISR |
| | | TPEVTCVVVDVSHEDP |
| | | EVKFNWYVDGVEVHNA |
| | | KTKPREEQYNSTYRVV |
| | | SVLTVLHQDWLNGKEY |
| | | KCKVSNKALPAPIEKT |
| | | ISKAKGQPREPQVYTL |
| | | PPSREEMTKNQVSLTC |
| | | LVKGFYPSDIAVEWES |
| | | NGQPENNYKTTPPVLD |
| | | SDGSFFLYSKLTVDKS |
| | | RWQQGNVFSCSVMHEA |
| | | LHNHYTQKSLSLSPGK |
| Mature cyno | 6 | QDGNEEMGSITQTPYQ |
| CD3e(1-49)_ | | VSISGTTVILTCDIKT |
| Fc2(K105-K330) | | HTCPPCPAPELLGGPS |
| | | VFLFPPKPKDTLMISR |
| | | TPEVTCVVVDVSHEDP |
| | | EVKFNWYVDGVEVHNA |
| | | KTKPREEQYNSTYRVV |
| | | SVLTVLHQDWLNGKEY |
| | | KCKVSNKALPAPIEKT |
| | | ISKAKGQPREPQVYTL |
| | | PPSREEMTKNQVSLTC |
| | | LVKGFYPSDIAVEWES |
| | | NGQPENNYKTTPPVLD |
| | | SDGSFFLYSKLTVDKS |
| | | RWQQGNVFSCSVMHEA |
| | | LHNHYTQKSLSLSPGK |
| Mature human | 7 | QDGNEEMGGITQTPYK |
| CD3e(1- | | VSISGTTVILTCPQYP |
| 118)_FLAG- | | GSEILWQHNDKNIGGD |
| chLys_Avi | | EDDKNIGSDEDHLSLK |
| | | EFSELEQSGYYVCYPR |
| | | GSKPEDANFYLYLRAR |
| | | VDIDYKDDDDKIEGRM |
| | | DKVFGRCELAAAMKRH |
| | | GLDNYRGYSLGNWVCA |
| | | AKFESNFNTQATNRNT |
| | | DGSTDYGILQINSRWW |
| | | CNDGRTPGSRNLCNIP |
| | | CSALLSSDITASVNCA |
| | | KKIVSDGNGMNAWVAW |
| | | RNRCKGTDVQAWIRGC |
| | | RLVNSRGLNDIFEAQK |
| | | IEWHE |
| Mature human | 8 | QDGNEEMGGITQTPYK |
| CD3e(1-118)_ | | VSISGTTVILTCPQYP |
| FLAG-chLys_ | | GSEILWQHNDKNIGGD |
| Avi-bio | | EDDKNIGSDEDHLSLK |
| | | EFSELEQSGYYVCYPR |
| | | GSKPEDANFYLYLRAR |
| | | VDIDYKDDDDKIEGRM |
| | | DKVFGRCELAAAMKRH |
| | | GLDNYRGYSLGNWVCA |
| | | AKFESNFNTQATNRNT |
| | | DGSTDYGILQINSRWW |
| | | CNDGRTPGSRNLCNIP |
| | | CSALLSSDITASVNCA |
| | | KKIVSDGNGMNAWVAW |
| | | RNRCKGTDVQAWIRGC |
| | | RLVNSRGLNDIFEAQK |
| | | IEWHE-BIOTIN |
| Mature human | 9 | QDGNEEMGGITQTPYK |
| CD3e(1-118)- | | VSISGTTVILTCPQYP |
| GS-PAS-CD3d | | GSEILWQHNDKNIGGD |
| (22-92)_FLAG- | | EDDKNIGSDEDHLSLK |
| chLys_Avi-bio | | EFSELEQSGYYVCYPR |
| | | GSKPEDANFYLYLRAR |
| | | VGGGGSGGGGSGGGGS |
| | | ASPAAFKIPIEELEDR |
| | | VFVNCNTSITWVEGTV |
| | | GTLLSDITRLDLGKRI |
| | | LDPRGIYRCNGTDIYK |
| | | DKESTVQVHYRMDIDY |
| | | KDDDDKIEGRMDKVFG |
| | | RCELAAAMKRHGLDNY |
| | | RGYSLGNWVCAAKFES |
| | | NFNTQATNRNTDGSTD |
| | | YGILQINSRWWCNDGR |
| | | TPGSRNLCNIPCSALL |
| | | SSDITASVNCAKKIVS |
| | | DGNGMNAWVAWRNRCK |
| | | GTDVQAWIRGCRLVNS |
| | | RGLNDIFEAQKIEWHE |
| | | -BIOTIN |
| Mature cyno | 10 | QDGNEEMGSITQTPYQ |
| CD3e(1-109)- | | VSISGTTVILTCSQHL |
| GS-PAS- | | GSEAQWQHNGKNKEDS |
| CD3d(22- | | GDRLFLPEFSEMEQSG |
| 92)_FLAG_ | | YYVCYPRGSNPEDASH |
| chLys_Avi | | HLYLKARVIDYKGGGG |
| | | SGGGGSGGGGSASPAA |
| | | FKIPVEELEDRVFVKC |
| | | NTSVTWVEGTVGTLLT |
| | | NNTRLDLGKRILDPRG |
| | | IYRCNGTDIYKDKESA |
| | | VQVHYRMDIDYKDDDD |
| | | KIEGRMDKVFGRCELA |
| | | AAMKRHGLDNYRGYSL |
| | | GNWVCAAKFESNFNTQ |
| | | ATNRNTDGSTDYGILQ |
| | | INSRWWCNDGRTPGSR |
| | | NLCNIPCSALLSSDIT |
| | | ASVNCAKKIVSDGNGM |
| | | NAWVAWRNRCKGTDVQ |
| | | AWIRGCRLVNSRGLND |
| | | IFEAQKIEWHE |
| Mature cyno | 11 | QDGNEEMGSITQTPYQ |
| CD3e(1-109)- | | VSISGTTVILTCSQHL |
| GS-PAS- | | GSEAQWQHNGKNKEDS |
| CD3d(22-92)_ | | GDRLFLPEFSEMEQSG |
| FLAG_chLys_ | | YYVCYPRGSNPEDASH |
| Avi-bio | | HLYLKARVIDYKGGGG |
| | | SGGGGSGGGGSASPAA |
| | | FKIPVEELEDRVFVKC |
| | | NTSVTWVEGTVGTLLT |
| | | NNTRLDLGKRILDPRG |
| | | IYRCNGTDIYKDKESA |
| | | VQVHYRMDIDYKDDDD |
| | | KIEGRMDKVFGRCELA |
| | | AAMKRHGLDNYRGYSL |
| | | GNWVCAAKFESNFNTQ |
| | | ATNRNTDGSTDYGILQ |
| | | INSRWWCNDGRTPGSR |
| | | NLCNIPCSALLSSDIT |
| | | ASVNCAKKIVSDGNGM |
| | | NAWVAWRNRCKGTDVQ |
| | | AWIRGCRLVNSRGLND |
| | | IFEAQKIEWHE- |
| | | BIOTIN |
| Mature cyno | 104 | QDGNEEMGSITQTPYQ |
| CD3e(1- | | VSISGTTVILTCSQHL |
| 108)_FLAG- | | GSEAQWQHNGKNKEDS |
| chLys_Avi | | GDRLFLPEFSEMEQSG |
| | | YYVCYPRGSNPEDASH |
| | | HLYLKARVVIDYKDDD |
| | | DKIEGRMDKVFGRCEL |
| | | AAAMKRHGLDNYRGYS |
| | | LGNWVCAAKFESNFNT |
| | | QATNRNTDGSTDYGIL |
| | | QINSRWWCNDGRTPGS |
| | | RNLCNIPCSALLSSDI |
| | | TASVNCAKKIVSDGNG |
| | | MNAWVAWRNRCKGTDV |
| | | QAWIRGCRLVNSRGLN |
| | | DIFEAQKIEWHE |

### Example 2: Generation of Fully Human Antibodies Binding to Human and Cynomolgus CD3 Epsilon from the MorphoSys Ylanthia® Library

For antibody generation, the MorphoSys Ylanthia® library was used to select Fab fragments against human and cynomolgus CD3. The MorphoSys Ylanthia® library (Tiller et al. mAbs 5:3, 1-26; May/June (2013) and U.S. Pat. No. 8,728,981) is a commercially available phagemid library and employs the CysDisplay® technology for displaying the Fab on the phage surface (Lohning et al., WO2001/05950).

To identify human/cynomolgus CD3 cross-reactive antibodies various panning strategies were performed. Thereby different synthetic and recombinant human and cynomolgus CD3 epsilon antigens representing the N-terminal extracellular region of CD3 epsilon were used. This region aligns to the binding region recognized by the human/cynomolgus CD3 epsilon cross-reactive prior-art antibody SP34. To identify CD3 specific antibodies, solution and semi-solution based panning strategies were performed using human and cynomolgus CD3 antigen material as indicated above to select species cross-reactive antibodies. Each conducted panning comprised of at least 3 individual rounds of selection.

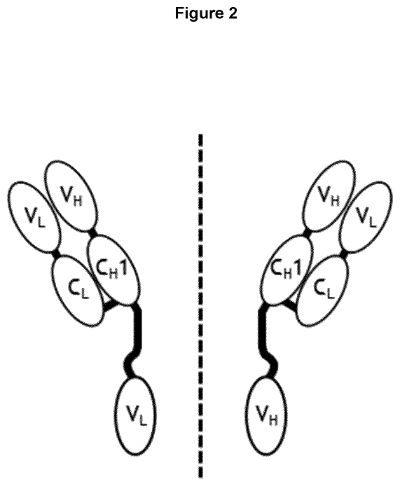

The identification of human/cynomolgus CD3 cross-reactive antibodies turned out to be very challenging, with the parental antibody MAB-1 being the only clone revealing specific binding to recombinant human and cynomolgus CD3 epsilon as well as cell binding to human and cynomolgus PBMCs (see FIG. 2). Surprisingly, MAB-1 was found suited to be used in the multispecific CyCAT format described herein.

MAB-1 was subjected to HCDR1+2 affinity maturation of the variable heavy chain (VH) and LCDR3 affinity maturation of the variable light chain (VL) using soluble CD3 epsilon antigens as described above and/or Jurkat cells with endogenous expression of human CD3 to further increase affinity and specificity for human and cynomolgus CD3 and to improve functional activity.

Accordingly, several hundred clones were screened for affinity. Functionality was rigorously tested in in vitro assays comprising e.g.
Binding to human T-cells and Jurkat cells with endogenous expression of CD3 and J.RT-T3.5 cells without CD3 expression in a HER2×CD3 bispecific antibody construct.
Functional NFAT Reporter Gene Assay in a HER2×CD3 bispecific antibody constructs
Functional cytotoxicity assay as HER2×CD3 bispecific constructs

Out of these studies, 7 most promising affinity matured variants of MAB-1 were further characterized for their suitably in the CyCAT format.

### Example 3: Identification of Improved Fully Human CD3 Specific Antibodies Suited for the CyCAT Format

In order to determine the suitability of the newly identified affinity matured CD3 specific human antibodies to be used in the CyCAT format, VH and VL domains of parental MAB-1, or of its affinity matured variants, as well as certain CD3-specific reference prior art antibodies were cloned into the set of polypeptides of the CyCAT format.

As exemplified herein, each polypeptide in the set of two polypeptides of the CyCAT format is composed of an antibody Fab fragment with specificity for the cancer target HER2 and either the VL or the VH domain of an anti-CD3 antibody of interest. The fusion between the Fab portion and the VL or VH domain is achieved by short peptide linkers between the C-terminus of the Fab heavy chain and the N-terminus of the CD3 specific VH or VL domain, respectively.

For HER2 binding, nucleotide sequences encoding the VH and VL domains from “Trastuzumab” (HERCEPTIN®) as described by Baselga et al. 1998, Cancer Res 58(13): 2825-2831) were used. Trastuzumab and its method of preparation are described in U.S. Pat. No. 5,821,337.

A summary of the protein sequences of the produced CyCAT polypeptides carrying the improved CD3 specific VH or VL domains made in accordance with the examples described herein are set forth in Tables: 6-7.

Gene Synthesis

All nucleic acid sequences or desired gene segments either were generated by PCR using appropriate templates or were gene synthesized as linear DNA fragments with appropriate flanking regions (e.g. suitable restriction enzyme recognition sites, linker sequences) in-house or by an external provider. The nucleic acid sequences or gene segments flanked by singular restriction endonuclease cleavage sites were cloned into respective mammalian expression vectors using standard molecular biology methods. When intended for use in mammalian expression vectors, all constructs were designed with a 5′-end DNA sequence coding for a leader peptide which targets proteins for secretion in eukaryotic cells. The DNA sequence of the subcloned gene fragments was confirmed by DNA by double strand sequencing

### Example 4: Recombinant Production of CyCAT Polypeptides

For expression of CyCAT polypeptides, exponentially growing eukaryotic HEK293-6E cells were transfected with a mammalian expression vector encoding all components of a CyCAT polypeptide, resulting in a 1:1 heterodimer of the polypeptide comprising the Fab heavy chain of Trastuzumab fused to the VH or VL domain of the CD3 antibody of interest and the Fab light chain of Trastuzumab, respectively.

Cell culture supernatants were harvested on day 6 post transfection and subjected to anti-CH1 affinity chromatography (Capture Select IgG-CH1 or CH1-XL|ThermoFisher Scientific). Buffer exchange was performed to 1× Dulbcecco's PBS (pH 7.2|Invitrogen) and samples were sterile filtered (0.2 μm pore size). Protein concentrations were determined by UV-spectrophotometry and purities of the constructs were analyzed under denaturing, reducing and non-reducing conditions using CE-SDS (LabChip GX Touch|Perkin Elmer|USA). UHP-SEC was performed to analyze individual unpaired CyCAT polypeptide preparations in native state.

Results:

VH and VL domains of 3 CD3 specific prior art antibodies (Ref-MAB-1, Ref-MAB-2, Ref-MAB-3) were produced as CyCAT polypeptides. Ref-MAB-2, Ref-MAB-3 are prior-art antibodies based on SP34 and are cross-reactive to cynomolgus CD3. Ref-MAB-2 was first described in WO2008/119567, Ref-MAB-2 was first described in WO2019/034580. Ref-MAB-1 binds a distinct epitope in CD3 and is not cross-reactive to cynomolgus CD3.

In addition, the VH and VL domain of the newly identified parental fully human antibody MAB-1 as well as 7 affinity matured variable domains of MAB-1 were produced as CyCAT polypeptides.

Results of the quality control of the mammalian produced CyCAT polypeptides are summarized in Table 12. This data reveal a strong CD3 specific antibody variable domain dependent producibility of the CyCAT polypeptides. In general, polypeptides carrying an unpaired CD3 specific VH domain appeared prone for aggregation when compared to polypeptides carrying an (unpaired) CD3 specific VL domain.

Overall, CyCAT polypeptides carrying the CD3 specific VH domains of the prior art reference antibodies (Ref-MAB-1, Ref-MAB-2, Ref-MAB-3) revealed poor producibility in terms of monomer content and purity. Surprisingly, both variable domains of the newly identified parental CD3 specific human antibody MAB-1 could be produced as unpaired CyCAT polypeptides with acceptable yield (data not shown), monomer content and purity (see Table 12). However, two produced polypeptides each carrying an affinity matured VH domain of MAB-1 (VH of MAB-2 and VH of MAB-3) failed in production, but 5 polypeptides carrying other affinity matured VH domains of MAB-1 (VH of MAB-4, MAB-5, MAB-6, and MAB-1_25) or the affinity matured VL domain of MAB-1 (VL of MAB-1_2) could be produced in an acceptable manner.

These results indicate the difficulty in finding suitable pairs of CD3 specific variable domains which can be actually produced in the CyCAT format.

TABLE 12
Summary quality control after production: Monomer content
and impurities of produced CyCAT polypeptides

| | | | | | SEC | |
|---|---|---|---|---|---|---|
| | | CyCAT polypeptides | Un- | | High | SEC Low |
| | | [Fab_Trastuzumab- | paired | SEC | molecular | molecular |
| | | CD3-VL] or | CD3 | Monomer | weight | weight |
| | | [Fab_Trastuzumab- | VH or | Content | impurities | impurities |
| Antibody | Source | CD3-VH] | VL | [%] | [%] | [%] |
| Ref-MAB-1 | Prior art | CyCAT_Ref-MAB-1_VL | VL | 89.3 | 10.6 | 0.2 |
| | | CyCAT_Ref-MAB-1_VH | VH | 78.9 | 10.4 | 10.7 |
| Ref-MAB-2 | Prior art | CyCAT_Ref-MAB-2_VL | VL | 87.9 | 12.1 | 0.0 |
| | | CyCAT_Ref-MAB-2_VH | VH | 81.9 | 11.2 | 7.0 |
| Ref-MAB-3 | Prior art | CyCAT_Ref-MAB-3_VL | VL | 92.4 | 4.7 | 3.1 |
| | | CyCAT_Ref-MAB-3_VH | VH | 88.0 | 11.4 | 0.6 |
| MAB-1 | parental | CyCAT_MAB-1_VL | VL | 95.1 | 4.4 | 0.4 |
| | | CyCAT_MAB-1_VH | VH | 82.9 | 16.4 | 0.7 |
| MAB-2 | LCDR3 | CyCAT_MAB-2_VL | VL | 81.6 | 10.7 | 10.7 |
| | maturation | | | | | |
| | HCDR1/2 | CyCAT_MAB-2_VH | VH | nep | nep | nep |
| | maturation | | | | | |
| MAB-3* | HCDR1/2 | CyCAT_MAB-3_VH | VH | nep | nep | nep |
| | maturation | | | | | |
| MAB-4* | HCDR1/2 | CyCAT_MAB-4_VH | VH | 85.9 | 9.1 | 5.1 |
| | maturation | | | | | |
| MAB-5* | HCDR1/2 | CyCAT_MAB-5_VH | VH | 80.7 | 10.3 | 9.1 |
| | maturation | | | | | |
| MAB-1_25* | HCDR1/2 | CyCAT_MAB-1_25_VH | VH | 78.6 | 19.1 | 2.3 |
| | maturation | | | | | |
| MAB-6* | HCDR1/2 | CyCAT_MAB-6_VH | VH | 78.1 | 15.2 | 6.8 |
| | maturation | | | | | |
| MAB-1_2** | LCDR3 | CyCAT_MAB-1_2_VL | VL | 96.3 | 3.3 | 0.4 |
| | maturation | | | | | |

*VH affinity matured descendants share the same VL of parental antibody MAB-1
**VL affinity matured descendant shares the same VH of parental antibody MAB-1
nep = no evaluation possible.

Produced CyCAT polypeptides carrying optimized CD3 specific variable domains derived from MAB-1 were then further characterized for affinity and functional activity in cytotoxicity assays as described herein in Example 10 and Example 11 by combining a CyCAT polypeptide carrying the VH of an CD3 specific antibody with a CyCAT polypeptide carrying the VL of an CD3 specific antibody.

Out of these studies, a new improved human CD3 specific antibody named MAB-7 was identified comprising the affinity matured VH domain of MAB-1_25 (MAB-1_25_VH (SEQ ID NO: 14)) and the affinity matured VL domain of MAB-1_2 (MAB-1_2_VL (SEQ ID NO: 17)).

An overview of the functional characteristics for the MAB-7 antibody (CyCAT-7) is shown in Table 15 (first row) and reveal that MAB-7 binds to human CD3 epsilon with an K_{D} of 5 nM and mediates efficient T-cell killing of HER-2 expressing SW-480 colon cancer cells when tested in the bispecific CyCAT format with IC50 values in the single digit nM range.

### Example 5: Identification of Further Improved Human Antibodies Specific for CD3

To identify further improved human CD3 specific variable domains of MAB-1 and MAB-7 in terms of affinity, potency and producibility in the CyCAT format, various strategies were pursued:
1. Additional LCDR1+2 affinity maturation of the variable light chain of MAB-7 (MAB-1_2_VL) using human and cynomolgus CD3 antigens, as well as Jurkat cells expressing native CD3.
2. Identification of new LCDR3 variants of parental antibody MAB-1 based on Next Generation Sequencing (NGS) via analysis of complete LCDR3 affinity maturation outputs from the first affinity maturation campaign described in Example 2.
3. Identification of new HCDR1+2 variants of parental MAB-1 based on NGS analysis of complete affinity maturation outputs from the first affinity maturation campaign described in Example 2.

Out of these studies, 81 most promising clones in terms of their e.g. affinity were produced as unpaired CyCAT polypeptides as described in Example 4.

### Example 6: Production of Unpaired CyCAT Polypeptides Carrying Improved CD3 Specific Variable Domains of Example 5

Out of the 81 clones of Example 5, variable domains of 57 affinity matured antibodies could be successfully produced as unpaired CyCAT polypeptides at least as good as the unpaired CyCAT polypeptides carrying either the VH domain of MAB-7 (MAB-1_25_VH; (SEQ ID NO: 14) or the VL domain of MAB-7 (MAB-1_2_VL; (SEQ ID NO: 17)). This included 6 of only 8 newly identified affinity matured VH domains.

Results:

Overall, production data revealed that a larger number of acidic amino acids in the CDRs of the CD3 specific variable domains were associated with better producibility of the CyCAT polypeptides. A summary of the quality control of produced CyCAT polypeptides carrying 13 improved CD3 specific VL domains (MAB-1_3_VL to MAB-1_15_VL) and 1 improved VH domain (MAB-1_24_VH) of parental MAB-1 is shown in Table 13.

These polypeptides could be produced in high yield (data not shown) and revealed excellent monomer content and purity.

In particular, the CyCAT polypeptides carrying the affinity matured VH domain of MAB-1_24 (MAB-1_24_VH; SEQ ID NO: 15) could be produced with a high monomer content of ˜90% which is significantly increased when compared to the formerly determined monomer content of its parental antibody MAB-1, which was only in the range of 80% (see Table 12).

MAB-1_24_VH was independently identified by a novel rational design approach and was based on the sequences of 100 unique affinity screening hits after H-CDR1+2 maturation. Amino acid frequency for each position of the affinity matured HCDR1+2 sequences was analyzed via Next Generation Sequencing. For each position the amino acid residue which showed best enrichment compared to the maturation module design was selected. If two or more amino acids were similarly enriched, acidic amino acids were preferred.

TABLE 13
Summary quality control of the produced CyCAT polypeptides carrying
further improved CD3 specific variable domains. Monomer content
and impurities of produced CyCAT polypeptides are shown.

| | | | | | SEC | SEC |
|---|---|---|---|---|---|---|
| | SEQ | | | | High | Low |
| Improved | ID | | SEQ ID | SEC | molecular | molecular |
| Anti-CD3 | NO: | | NO: | Monomer | weight | weight |
| Variable | CD3 | CyCAT | CyCAT | Content | impurities | impurities |
| Domain | VH/VL | polypeptide | polypept. | [%] | [%] | [%] |
| MAB-1_3_VL | 18 | CyCAT_MAB- | 65 | 94.1 | 5.4 | 0.6 |
| | | 1_3_VL | | | | |
| MAB-1_4_VL | 19 | CyCAT_MAB- | 66 | 96.0 | 3.7 | 0.3 |
| | | 1_4_VL | | | | |
| MAB-1_5_VL | 20 | CyCAT_MAB- | 67 | 90.0 | 9.7 | 0.3 |
| | | 1_5_VL | | | | |
| MAB-1_6_VL | 21 | CyCAT_MAB- | 68 | 90.9 | 8.4 | 0.7 |
| | | 1_6_VL | | | | |
| MAB-1_7_VL | 22 | CyCAT_MAB- | 69 | 97.1 | 2.5 | 0.4 |
| | | 1_7_VL | | | | |
| MAB-1_8_VL | 23 | CyCAT_MAB- | 70 | 95.3 | 4.2 | 0.5 |
| | | 1_8_VL | | | | |
| MAB-1_9_VL | 24 | CyCAT_MAB- | 71 | 97.2 | 2.8 | 0.0 |
| | | 1_9_VL | | | | |
| MAB-1_10_VL | 25 | CyCAT_MAB- | 72 | 96.9 | 3.0 | 0.1 |
| | | 1_10_VL | | | | |
| MAB-1_11_VL | 26 | CyCAT_MAB- | 73 | 95.3 | 4.0 | 0.8 |
| | | 1_11_VL | | | | |
| MAB-1_12_VL | 27 | CyCAT_MAB- | 74 | 94.4 | 5.6 | 0.0 |
| | | 1_12_VL | | | | |
| MAB-1_13_VL | 28 | CyCAT_MAB- | 75 | 92.2 | 7.3 | 0.5 |
| | | 1_13_VL | | | | |
| MAB-1_14_VL | 29 | CyCAT_MAB- | 76 | 94.1 | 5.9 | 0.0 |
| | | 1_14_VL | | | | |
| MAB-1_15_VL | 30 | CyCAT_MAB- | 77 | 96.1 | 3.1 | 0.8 |
| | | 1_15_VL | | | | |
| MAB-1_24_VH | 15 | CyCAT_MAB- | 88 | 90.2 | 9.5 | 0.3 |
| | | 1_24_VH | | | | |

### Example 7: Characterization of Paired CyCAT Polypeptides Carrying Improved CD3 Specific Variable Domains of Examples 5 and 6

52 successfully produced CyCAT polypeptides of Example 6 were subjected to affinity determination as described in Example 10 and functional testing as described in Example 11 by combing them with CyCAT polypeptides carrying either the VH of MAB-7 (MAB-1_25_VH (SEQ ID NO: 14)) or the VL of MAB-7 (MAB-1_2_VL (SEQ ID NO: 17)).

These studies resulted in the identification of 13 improved and most preferred CD3 specific VL domains and 1 CD3 specific VH domain derived from of MAB-1 or MAB-7 of Example 6 (as summarized in Table 13).

An overview of the functional characteristics of the preferred combinations of the CD3 specific VL and VH domains are summarized in Table 15 and reveal that the newly identified CD3 specific antibodies according to the present disclosure bind to human CD3 epsilon with an K_{D} of 5 nM or lower and mediate efficient T cell killing of SW-480 colon cancer cells in the CyCAT format with IC₅₀ values mostly in the subnanomolar range.

Out of these VH/VL combinations, MAB-7, MAB-8, MAB-9, MAB-12, MAB-13 and MAB-16 were selected as the CD3 specific lead antibodies for use in T-cell engaging multispecific antibody formats.

### Example 8: Production of Unpaired CyCAT Polypeptides Carrying Further Improved CD3 Specific Variable Domains of MAB-1 or MAB-7

As a final step of optimization, 8 cross-clones were generated by combining the LCDR1+2 sequences of the best LCDR1+2 affinity matured variants of MAB-7 (see Table 13) with the LCDR3 sequence of MAB-1_14_VL (SEQ ID NO: 29). MAB-1_14_VL was identified as described in Example 5 (option 2 of the numbered list) as a consensus sequence based on the enrichment of particular amino acid residues at certain positions in the LCDR3 when compared to the design of the affinity maturation module used. CyCAT polypeptides where then produced as described in Example 4.

Results

A summary of the quality control of the produced CyCAT polypeptides carrying the further improved CD3 specific VL domains of the 8 cross-clones is shown in Table 14. These produced polypeptides revealed an overall less favorable monomer content and a higher degree of high molecular weight impurities after production when compared the polypeptides carrying the improved CD3 specific variable domains of Table 15. Therefore, the polypeptides were subjected to a preparatory size exclusion chromatography (Prep-SEC). Prep-SEC preparations resulted in a very high monomer content for the CyCAT polypeptides as shown in Table 14.

TABLE 14
Summary quality control of produced CyCAT polypeptides carrying further
improved cross-cloned CD3 specific variable light chain domains before
and after preparatory size exclusion chromatography. Monomer content
and impurities of produced CyCAT polypeptides are shown.

| Further | | | | | SEC | SEC | |
|---|---|---|---|---|---|---|---|
| improved | | | | | High | Low | Polished |
| Anti-CD3 | | | | SEC | molecular | molecular | SEC |
| Variable | SEQ | | SEQ | Monomer | weight | weight | Monomer |
| Domain | ID | CyCAT | ID | Content | impurities | impurities | Content |
| (X-clones) | NO: | polypeptide | NO: | [%]* | [%] | [%] | [%]* |
| MAB- | 31 | CyCAT_MAB- | 78 | 89.8 | 10.2 | 0.0 | 97.0 |
| 1_16_VL | | 1_16_VL | | | | | |
| MAB- | 32 | CyCAT_MAB- | 79 | nep* | nep* | nep* | 99.0 |
| 1_17_VL | | 1_17_VL | | | | | |
| MAB- | 33 | CyCAT_MAB- | 80 | 83.7 | 14.8 | 1.5 | 99.7 |
| 1_18_VL | | 1_18_VL | | | | | |
| MAB- | 34 | CyCAT_MAB- | 81 | 92.4 | 7.6 | 0.0 | 99.0 |
| 1_19_VL | | 1_19_VL | | | | | |
| MAB- | 35 | CyCAT_MAB- | 82 | nep* | nep* | nep* | 99.2 |
| 1_20_VL | | 1_20_VL | | | | | |
| MAB- | 36 | CyCAT_MAB- | 83 | 84.9 | 13.7 | 1.4 | 99.4 |
| 1_21_VL | | 1_21_VL | | | | | |
| MAB- | 37 | CyCAT_MAB- | 84 | 81.3 | 17.1 | 1.5 | 92.9 |
| 1_22_VL | | 1_22_VL | | | | | |
| MAB- | 38 | CyCAT_MAB- | 85 | 90.1 | 9.9 | 0.0 | 99.2 |
| 1_23_VL | | 1_23_VL | | | | | |

nep* = no evaluation possible

### Example 9: Characterization of Paired CyCAT Polypeptides Carrying Improved Cross-Cloned CD3 Specific Variable Light Chain Domains of Examples 8

The produced CyCAT polypeptides of Example 8 were subjected to affinity determination as described in Example 10 and functional testing as described in Example 11 by combing them with the CyCAT polypeptides carrying the VH domain of MAB-7 (MAB-1_25_VH (SEQ ID NO: 14)) or the newly identified optimized VH domain of MAB-1_24 (MAB-1_24_VH (SEQ ID NO: 15)).

An overview of the functional characteristics of the further improved CD3 specific VL and VH domains are summarized in Table 16. This data reveal that the paired CyCAT polypeptides comprising the further improved CD3 specific VL domains bind to human CD3 epsilon with an K_{D} of below 1 nM. However, no further improved T-cell mediated killing of SW-480 colon cancer cells in the CyCAT format could be observed (data not shown).

### Example 10: Affinity Determination for Paired CD3 VH/VL Domains in the CyCAT Format on Human CD3 Epsilon

Produced unpaired CyCAT polypeptides carrying either a VH or VL domain of an CD3 specific antibody according to the present disclosure were combined to allow for the formation of paired and functional CD3 specific Fv domains. Functional complementation of the CD3-specific VH and VL domains occurs once each of the two CyCAT polypeptides is bound to its target antigen via the Fab portion and the two polypeptides come in close proximity.

Method

K_{D} determination for paired VH and VL domains was performed on an Octet HTX (FortéBIO, Sartorius AG) instrument at 27° C.

Streptavidin-coated biosensors (FortéBIO, Sartorius AG) were loaded with purified and biotinylated human HER2_his_Avi_bio (ACROBiosystems, HE2-H82E2) in immobilization buffer (DPBS, 0.05% (v/v) Tween 20) resulting in a high density human HER2 sensor surface. Afterwards, CyCAT polypeptides were diluted in Octet buffer (DPBS, 0.05% (v/v) Tween-20, 0.1% (w/v) BSA) and sequentially injected until a loading threshold value of approx. 0.5 nm was reached for CyCAT_VH_CD3 and CyCAT_VL_CD3, respectively. Kinetic measurements were done using 3 different human CD3 epsilon (CD3e (1-118)_FLAG_chLys_AVI-bio (MorphoSys AG) (SEQ ID NO: 8) concentrations (4-fold serial dilution, 120 to 7.5 nM in Octet buffer) with 300 s association time and 720 s dissociation time. After each dissociation step the sensors were regenerated to remove bound CyCAT molecules (2×30 s Gly/HCl, pH 1.5).

All data were referenced with Streptavidin-coated biosensors loaded with human HER2_his_Avi_bio incubated in Octet buffer instead of analyte containing buffer. The sensorgrams were fitted using Octet Data Analysis Software 10.0 (FortéBio, Sartorius AG) to determine kon and koff rate constants (using a 1:1 binding model), which were used to calculate KD.

Results

A summary of the determined affinities on human CD3 epsilon for preferred VH and VL pairs is provided in Table 15 and Table 16 and reveal monovalent affinities for the newly identified CD3 specific antibodies according to the present disclosure on human CD3 epsilon in the single digit nanomolar to triple digit picomolar range.

As expected, testing of unpaired not combined CyCAT polypeptides of CyCAT_MAB-1_24_VH, CyCAT_MAB-1_3_VL, CyCAT_MAB-1_14_VL and CyCAT_MAB-1_16_VL to CyCAT_MAB-1_23_VL up to a concentration of 500 nM revealed no binding to CD3 epsilon due to lack of formation of a functional CD3 specific Fv binding fragment.

### Example 11: T Cell Mediated Killing of Cancer Cells Induced by Paired CyCAT Polypeptides

The produced unpaired CyCAT polypeptides were combined to allow for the formation of a CyCAT molecule comprising a newly formed CD3 specific Fv binding domain. Functional complementation of the CD3 specific VH and VL domains occurs once each of the two CyCAT polypeptides is bound via its Fab portion to its target antigen on the cancer cell and the two polypeptides are in close proximity. Redirected killing of cancer cells via immune cells is mediated by binding of the newly formed CD3 specific antibody Fv domain to T cells.

Methods

Isolation of Human T-Cells

Human whole blood from healthy donors was collected e.g. in Li-Heparin containing S-Monovette containers (Sarstedt). 20 mL blood were transferred to 50 mL conical tubes, mixed with 1 mL of RosetteSep Human CD8+ Enrichment Cocktail (Stemcell Technologies, #15063) and incubated for 20 min at room temperature. Blood containing RosetteSep human CD8+ enrichment cocktail was diluted with an equal volume of PBS containing 2% fetal bovine serum (Sigma, #F7524) and 2 mM EDTA. Diluted blood was transferred to SepMate-50 tubes (Stemcell Technologies, #85450) containing 15 mL of Lymphoprep density gradient medium (Stemcell Technologies, #07811) and centrifuged for 20 min at 1200×g at room temperature. Supernatant was transferred into a 50 mL conical tube, diluted to 45 mL with PBS containing 2% fetal bovine serum and 2 mM EDTA and centrifuged for 5 min at 800×g. Supernatant was discarded, cell pellet resuspended in 1 mL PBS containing 2% fetal bovine serum. Cell suspensions were pooled and transferred to a 50 mL tube and diluted to 30 mL PBS containing 2% fetal bovine serum. Cells were pelleted by centrifugation for 5 min at 800×g. The cell pellet was resuspended in 2 mL of 1×Pharm Lyse Red Blood Cell lysing buffer (BD, #555899) and incubated at 4° C. for 10 min. PBS containing 2% fetal bovine serum was added to a final volume of 15 mL. Cells were pelleted for 10 min at 120×g and the supernatant decanted. The cells were washed twice with PBS containing 2% fetal bovine serum and counted (CASY TT device, Beckmann Coulter).

Assay Method

7,5000 HER-2 expressing SW-480 colon cancer cells (ATCC® CCL-228™) were suspended in culture medium supplemented with 10% FCS, seeded in black 96 well assay plates (Corning) and incubated over night at 37° C. and 5% CO₂ and humidity.

CellToxGreen dye (Promega, #G8731), serially diluted CyCAT polypeptides (final concentration: 0.00001-100 nM) and purified human T cells (E:T ratio 1:10) or human PBMCs (E:T ratio of 1:30), all diluted in assay medium comprising RPMI 1640 w/o Phenol red (Gibco, #32404-014), GlutaMAX (Gibco 35050-038) and 10% fetal bovine serum, were added to the cells and incubated for 48 or 72 hrs at 37° C. and 5% CO₂ and humidity.

Cytotoxic activity was assessed by measuring incorporated CellToxGreen fluorescence at 485 nm excitation and 535 nm emission using a Tecan Infinite F500 device.

Results

The results of the experiments for selected CyCAT polypeptides carrying the newly identified variable domains of the CD3 specific antibodies according of the present disclosure are summarized in Table 15.

Co-cultivation of T-cells with a combination of CyCAT polypeptides with specificity for HER-2 and CD3 induced killing of HER-2 positive SW-480 target cells in a dose dependent manner. As expected, a negative control combination of CyCAT polypeptides induced no killing activity (data not shown). Co-cultivation of T-cells with unpaired and not combined CyCAT polypeptide with specificity for HER-2 and CD3 did not induced killing of HER-2 positive SW480 target cells. In the presence of HER-2 negative cells, none of the tested HER-2 specific combinations of CyCAT polypeptides induced cytotoxic activity (data not shown).

These results clearly demonstrate that the CD3 specific VH and VL domains comprised in each of the unpaired CyCAT polypeptides are complemented to a functional CD3 specific antibody Fv domain once both CyCAT polypeptides are bound via their Fab portion to the target antigen on the cancer cell and the two polypeptides get in close proximity. Accordingly, the newly identified CD3 specific human antibodies according to the present disclosure can be used in various approaches to efficiently re-direct T-cells to kill tumor expressing cells.

Summary Affinities and Functional Activity of the New CD3 Specific Antibodies According to the Present Disclosure.

TABLE 15
Summary affinities of CD3 specific antibodies (according Examples 5 to 7) and re-directed T-cell
killing of cancer cells by corresponding CyCAT molecules with specificity for HER2 and CD3.

| | | | | | | | T cell | T cell |
|---|---|---|---|---|---|---|---|---|
| | | | New | | | | Killing | Killing |
| | | | anti-CD3 antibody | | | Affinity | 48 h | 72 h |
| | | | | | Anti- | CD3 | IC₅₀ | IC₅₀ |
| | | SEQ | | SEQ | CD3 | K_{D} [nM] | [nM] | [nM] |
| | Paired CyCAT | ID | CD3_VL | ID | Anti- | hCD3e | SW- | SW- |
| CyCAT | polypeptides | NO: | CD3_VH | NO: | body | (1-118) | 480 | 480 |
| CYCAT-7 | CyCAT_MAB- | 64 | MAB- | 17 | MAB-7 | 5 | 2.72 | 0.54 |
| | 1_2_VL | | 1_2_VL | | | | 5.70 | 0.43 |
| | CyCAT_MAB- | 89 | MAB- | 14 | | | N/A | N/A |
| | 1_25_VH | | 1_25_VH | | | | 17.46 | 9.03 |
| CYCAT-8 | CyCAT_MAB- | 64 | MAB- | 17 | MAB-8 | 1.4 | 0.80 | 0.35 |
| | 1_2_VL | | 1_2_VL | | | | 0.93 | 0.61 |
| | CyCAT_MAB- | 88 | MAB- | 15 | | | | |
| | 1_24_VH | | 1_24_VH | | | | | |
| CYCAT-9 | CyCAT_MAB- | 65 | MAB- | 18 | MAB-9 | 0.2 | 0.54 | 0.32 |
| | 1_3_VL | | 1_3_VL | | | 0.7 | 1.70 | 1.07 |
| | CyCAT_MAB- | 88 | MAB- | 15 | | | | |
| | 1_24_VH | | 1_24_VH | | | | | |
| CYCAT-10 | CyCAT_MAB- | 66 | MAB- | 19 | MAB-10 | 1.2 | 0.36 | 0.28 |
| | 1_4_VL | | 1_4_VL | | | | 3.19 | 1.37 |
| | CyCAT_MAB- | 88 | MAB- | 15 | | | | |
| | 1_24_VH | | 1_24_VH | | | | | |
| CYCAT-11 | CyCAT_MAB- | 70 | MAB- | 23 | MAB-11 | 1.1 | 1.09 | 0.67 |
| | 1_8_VL | | 1_8_VL | | | | 1.59 | 0.85 |
| | CyCAT_MAB- | 88 | MAB- | 15 | | | | |
| | 1_24_VH | | 1_24_VH | | | | | |
| CYCAT-12 | CyCAT_MAB- | 71 | MAB- | 24 | MAB-12 | 0.8 | 0.30 | 0.23 |
| | 1_9_VL | | 1_9_VL | | | | 1.40 | 0.98 |
| | CyCAT_MAB- | 88 | MAB- | 15 | | | | |
| | 1_24_VH | | 1_24_VH | | | | | |
| CYCAT-13 | CyCAT_MAB- | 72 | MAB- | 25 | MAB-13 | 0.9 | 0.46 | 0.28 |
| | 1_10_VL | | 1_10_VL | | | | 1.36 | 0.84 |
| | CyCAT_MAB- | 88 | MAB- | 15 | | | | |
| | 1_24_VH | | 1_24_VH | | | | | |
| CYCAT-14 | CyCAT_MAB- | 73 | MAB- | 26 | MAB-14 | 1.2 | 1.43 | 0.57 |
| | 1_11_VL | | 1_11_VL | | | | 2.95 | 1.28 |
| | CyCAT_MAB- | 88 | MAB- | 15 | | | | |
| | 1_24_VH | | 1_24_VH | | | | | |
| CYCAT-15 | CyCAT_MAB- | 74 | MAB- | 27 | MAB-15 | 0.3 | 0.58 | 0.42 |
| | 1_12_VL | | 1_12_VL | | | | 1.29 | 0.91 |
| | CyCAT_MAB- | 88 | MAB- | 15 | | | | |
| | 1_24_VH | | 1_24_VH | | | | | |
| CYCAT-16 | CyCAT_MAB- | 76 | MAB- | 29 | MAB-16 | 0.9 | 0.33 | 0.22 |
| | 1_14_VL | | 1_14_VL | | | 0.9 | 0.35 | 0.23 |
| | CyCAT_MAB- | 88 | MAB- | 15 | | | 0.56 | 0.33 |
| | 1_24_VH | | 1_24_VH | | | | | |
| CYCAT-17 | CyCAT_MAB- | 65 | MAB- | 18 | MAB-17 | 1.1 | 1.91 | 0.59 |
| | 1_3_VL | | 1_3_VL | | | | 20.53 | 2.40 |
| | CyCAT_MAB- | 89 | MAB- | 14 | | | | |
| | 1_25_VH | | 1_25_VH | | | | | |
| CYCAT-18 | CyCAT_MAB- | 74 | MAB- | 27 | MAB-18 | 1.6 | 1.56 | 0.45 |
| | 1_12_VL | | 1_12_VL | | | | | |
| | CyCAT_MAB- | 89 | MAB- | 14 | | | | |
| | 1_25_VH | | 1_25_VH | | | | | |
| CYCAT-19 | CyCAT_MAB- | 76 | MAB- | 29 | MAB-19 | 2.6 | 15.36 | 0.37 |
| | 1_14_VL | | 1_14_VL | | | | | |
| | CyCAT_MAB- | 89 | MAB- | 14 | | | | |
| | 1_25_VH | | 1_25_VH | | | | | |

TABLE 16
Summary affinities of CD3 specific antibodies with cross-
cloned variable light chain domains according Example 8.

| | Paired | | | | | |
|---|---|---|---|---|---|---|
| | CyCAT | | | | | |
| | polypeptides | | New | | | Affinity |
| | [Fab_Trastuzumab]- | | anti-CD3 antibody | | | CD3 |
| | Linker-[CD3-VL] x | SEQ | | SEQ | CD3 | K_{D} [nM] |
| | [Fab_Trastuzumab]- | ID | CD3_VL | ID | body | hCD3e |
| CyCAT | Linker-[CD3-VH] | NO: | CD3_VH | NO: | anti- | (1-118) |
| CYCAT-20 | CyCAT_MAB-1_16_VL | 78 | MAB-1_16_VL | 31 | MAB-20 | 0.5 |
| | CyCAT_MAB-1_24_VH | 88 | MAB-1_24_VH | 15 | | |
| CYCAT-21 | CyCAT_MAB-1_17_VL | 79 | MAB-1_17_VL | 32 | MAB-21 | 0.4 |
| | CyCAT_MAB-1_24_VH | 88 | MAB-1_24_VH | 15 | | |
| CYCAT-22 | CyCAT_MAB-1_18_VL | 80 | MAB-1_18_VL | 33 | MAB-22 | 0.6 |
| | CyCAT_MAB-1_24_VH | 88 | MAB-1_24_VH | 15 | | |
| CYCAT-23 | CyCAT_MAB-1_19_VL | 81 | MAB-1_19_VL | 34 | MAB-23 | 0.4 |
| | CyCAT_MAB-1_24_VH | 88 | MAB-1_24_VH | 15 | | |
| CYCAT-24 | CyCAT_MAB-1_20_VL | 82 | MAB-1_20_VL | 35 | MAB-24 | 0.6 |
| | CyCAT_MAB-1_24_VH | 88 | MAB-1_24_VH | 15 | | |
| CYCAT-25 | CyCAT_MAB-1_21_VL | 83 | MAB-1_21_VL | 36 | MAB-25 | 0.3 |
| | CyCAT_MAB-1_24_VH | 88 | MAB-1_24_VH | 15 | | |
| CYCAT-26 | CyCAT_MAB-1_22_VL | 84 | MAB-1_22_VL | 37 | MAB-26 | 0.1* |
| | CyCAT_MAB-1_24_VH | 88 | MAB-1_24_VH | 15 | | |
| CYCAT-27 | CyCAT_MAB-1_23_VL | 85 | MAB-1_23_VL | 38 | MAB-27 | 0.5 |
| | CyCAT_MAB-1_24_VH | 88 | MAB-1_24_VH | 15 | | |

*KD restriction; dissociation reached assay limit

### Example 12: Reconversion and Production of Newly Identified and Optimized CD3 Specific VH/VL Pairs into Conventional Fab and IgG Antibody Backbones

In order to determine the suitability of the newly identified and optimized CD3 specific VH/VL pairs to be used in conventional antibody formats, most preferred VH/VL pairs (see Table 17) were cloned into the mammalian IgG1f-AEASS (effector silent) or FabCys expression vectors for IgG or Fab production, respectively.

All nucleic acid sequences or desired gene segments were generated by PCR using appropriate templates or were gene synthesized as linear DNA fragments with appropriate flanking regions for Gibson Assembly, (e.g. suitable restriction enzyme recognition sites and 21 bp overlap sequence) in-house or by an external provider. The nucleic acid sequences were cloned into respective Ylanthia® FabCys mammalian expression vectors.

For IgG expression, antibody variable domain encoding FabCys vectors were enzymatically digested and the resulting inserts were ligated with the Ylanthia® mammalian expression cassette and further sub-cloned into the respective mammalian human silent IgG vectors.

TABLE 17
Selected optimized CD3 specific human antibodies re-converted from
the CyCAT into the Fab-Cys and the human IgG1-AEASS format.

| Anti-CD3 | | |
|---|---|---|
| Antibody | VH/VL domains | SEQ ID NO: |
| MAB-7 | MAB-1_2_VL | 17 |
| | MAB1_25_VH | 14 |
| MAB-8 | MAB-1_2_VL | 17 |
| | MAB-1_24_VH | 15 |
| MAB-9 | MAB-1_3_VL | 18 |
| | MAB-1_24_VH | 15 |
| MAB-12 | MAB-1_9_VL | 24 |
| | MAB-1_24_VH | 15 |
| MAB-13 | MAB-1_10_VL | 25 |
| | MAB-1_24_VH | 15 |
| MAB-16 | MAB-1_14_VL | 29 |
| | MAB-1_24_VH | 15 |

TABLE 18
Human FabCys and human IgG1f-AEASS
amino acid sequences of produced
CD3 specific antibodies according
to the present disclosure.

| | | SEQ | |
|---|---|---|---|
| | | ID | |
| Antibody# | | NO: | [aa] |
| MAB-7 | h_IgG1-AEASS | 95 | EVQLVESGGGLVQPGGSLRL |
| | Heavy chain | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSCDKTHTCPPCPAPEA |
| | | | EGAPSVFLFPPKPKDTLMIS |
| | | | RTPEVTCVVVDVSHEDPEVK |
| | | | FNWYVDGVEVHNAKTKPREE |
| | | | QYNSTYRVVSVLTVLHQDWL |
| | | | NGKEYKCKVSNKALPSSIEK |
| | | | TISKAKGQPREPQVYTLPPS |
| | | | REEMTKNQVSLTCLVKGFYP |
| | | | SDIAVEWESNGQPENNYKTT |
| | | | PPVLDSDGSFFLYSKLTVDK |
| | | | SRWQQGNVFSCSVMHEALHN |
| | | | HYTQKSLSLSPGK |
| | h_IgG1f Light | 96 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys | 97 | EVQLVESGGGLVQPGGSLRL |
| | Heavy chain | | SCAASGFTFKSYYMSWVRQA |
| | | | PGKGLEWVANIDYQSQHAYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 96 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| MAB-8 | h_IgG1-AEASS | 98 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSCDKTHTCPPCPAPEA |
| | | | EGAPSVFLFPPKPKDTLMIS |
| | | | RTPEVTCVVVDVSHEDPEVK |
| | | | FNWYVDGVEVHNAKTKPREE |
| | | | QYNSTYRVVSVLTVLHQDWL |
| | | | NGKEYKCKVSNKALPSSIEK |
| | | | TISKAKGQPREPQVYTLPPS |
| | | | REEMTKNQVSLTCLVKGFYP |
| | | | SDIAVEWESNGQPENNYKTT |
| | | | PPVLDSDGSFFLYSKLTVDK |
| | | | SRWQQGNVFSCSVMHEALHN |
| | | | HYTQKSLSLSPGK |
| | h_IgG1f Light | 96 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys Heavy | 99 | EVQLVESGGGLVQPGGSLRL |
| | Chain | | SCAASGFTFRSHYMTWVRQA |
| | | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 96 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| MAB-9 | h_IgG1-AEASS | 98 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSCDKTHTCPPCPAPEA |
| | | | EGAPSVFLFPPKPKDTLMIS |
| | | | RTPEVTCVVVDVSHEDPEVK |
| | | | FNWYVDGVEVHNAKTKPREE |
| | | | QYNSTYRVVSVLTVLHQDWL |
| | | | NGKEYKCKVSNKALPSSIEK |
| | | | TISKAKGQPREPQVYTLPPS |
| | | | REEMTKNQVSLTCLVKGFYP |
| | | | SDIAVEWESNGQPENNYKTT |
| | | | PPVLDSDGSFFLYSKLTVDK |
| | | | SRWQQGNVFSCSVMHEALHN |
| | | | HYTQKSLSLSPGK |
| | h_IgG1f Light | 100 | QSVLTQPPSASGTPGQRVTI |
| | Chain | | SCSGSSSNIGINYVYWYQQL |
| | (VL + CL) | | PGTAPKLLIYRNNIRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys | 99 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 100 | QSVLTQPPSASGTPGQRVTI |
| | Chain | | SCSGSSSNIGINYVYWYQQL |
| | (VL + CL) | | PGTAPKLLIYRNNIRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| MAB-12 | h_IgG1-AEASS | 98 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSS |
| | | | LGTQTYICNVNHKPSNTKVD |
| | | | KRVEPKSCDKTHTCPPCPAP |
| | | | EAEGAPSVFLFPPKPKDTLM |
| | | | ISRTPEVTCVVVDVSHEDPE |
| | | | VKFNWYVDGVEVHNAKTKPR |
| | | | EEQYNSTYRVVSVLTVLHQD |
| | | | WLNGKEYKCKVSNKALPSSI |
| | | | EKTISKAKGQPREPQVYTLP |
| | | | PSREEMTKNQVSLTCLVKGF |
| | | | YPSDIAVEWESNGQPENNYK |
| | | | TTPPVLDSDGSFFLYSKLTV |
| | | | DKSRWQQGNVFSCSVMHEAL |
| | | | HNHYTQKSLSLSPGK |
| | h_IgG1f Light | 101 | QSVLTQPPSASGTPGQRVTI |
| | Chain | | SCSGSSSNIGANYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys | 99 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 101 | QSVLTQPPSASGTPGQRVTI |
| | Chain | | SCSGSSSNIGANYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| MAB-13 | h_IgG1-AEASS | 98 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSCDKTHTCPPCPAPEA |
| | | | EGAPSVFLFPPKPKDTLMIS |
| | | | RTPEVTCVVVDVSHEDPEVK |
| | | | FNWYVDGVEVHNAKTKPREE |
| | | | QYNSTYRVVSVLTVLHQDWL |
| | | | NGKEYKCKVSNKALPSSIEK |
| | | | TISKAKGQPREPQVYTLPPS |
| | | | REEMTKNQVSLTCLVKGFYP |
| | | | SDIAVEWESNGQPENNYKTT |
| | | | PPVLDSDGSFFLYSKLTVDK |
| | | | SRWQQGNVFSCSVMHEALHN |
| | | | HYTQKSLSLSPGK |
| | h_IgG1f Light | 102 | QSVLTQPPSASGTPGQRVTI |
| | Chain | | SCSGSSSNIGINYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys | 99 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 102 | QSVLTQPPSASGTPGQRVTI |
| | Chain | | SCSGSSSNIGINYVYWYQQL |
| | (VL + CL) | | PGTAPKLLIYRNNQRPSGVP |
| | | | DRFSGSKSGTSASLAISGLR |
| | | | SEDEADYYCAAWDRHSHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| MAB-16 | h_IgG1-AEASS | 98 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSCDKTHTCPPCPAPEA |
| | | | EGAPSVFLFPPKPKDTLMIS |
| | | | RTPEVTCVVVDVSHEDPEVK |
| | | | FNWYVDGVEVHNAKTKPREE |
| | | | QYNSTYRVVSVLTVLHQDWL |
| | | | NGKEYKCKVSNKALPSSIEK |
| | | | TISKAKGQPREPQVYTLPPS |
| | | | REEMTKNQVSLTCLVKGFYP |
| | | | SDIAVEWESNGQPENNYKTT |
| | | | PPVLDSDGSFFLYSKLTVDK |
| | | | SRWQQGNVFSCSVMHEALHN |
| | | | HYTQKSLSLSPGK |
| | h_IgG1f Light | 103 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAGWDRRLHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |
| | FabCys | 99 | EVQLVESGGGLVQPGGSLRL |
| | Heavy | | SCAASGFTFRSHYMTWVRQA |
| | Chain | | PGKGLEWVANIDYEGTRTYY |
| | | | AESVKGRFTISRDNAKNSLY |
| | | | LQMNSLRAEDTAVYYCARGY |
| | | | SAEFAHRSGLDVWGQGTLVT |
| | | | VSSASTKGPSVFPLAPSSKS |
| | | | TSGGTAALGCLVKDYFPEPV |
| | | | TVSWNSGALTSGVHTFPAVL |
| | | | QSSGLYSLSSVVTVPSSSLG |
| | | | TQTYICNVNHKPSNTKVDKR |
| | | | VEPKSC |
| | FabCys Light | 103 | QSVLTQPPSASGTPGQRVTI |
| | Chain | | SCSGSSSNIGSNYVYWYQQL |
| | (VL + CL) | | PGTAPKLLIYRNNQRPSGVP |
| | | | DRFSGSKSGTSASLAISGLR |
| | | | SEDEADYYCAGWDRRLHGAV |
| | | | FGGGTKLTVLGQPKAAPSVT |
| | | | LFPPSSEELQANKATLVCLI |
| | | | SDFYPGAVTVAWKADSSPVK |
| | | | AGVETTTPSKQSNNKYAASS |
| | | | YLSLTPEQWKSHRSYSCQVT |
| | | | HEGSTVEKTVAPTECS |

Exploratory Scale Production of IgG

Eukaryotic HEK293 cells were transfected with mammalian expression vector DNA encoding both heavy and light chains of IgG. Cell culture supernatants were harvested on day 3 post transfection and subjected to standard Protein A affinity chromatography (MabSelect SURE|GE Healthcare). If not stated otherwise, buffer exchange was performed to 1× Dulbcecco's PBS (pH 7.2|Invitrogen) and samples were sterile filtered (0.2 μm pore size).

Protein concentrations were determined by UV-spectrophotometry and purity of IgG was analyzed under denaturing, reducing and non-reducing conditions using CE-SDS (LabChip GXII|Perkin Elmer|USA). HP-SEC was performed to analyze IgG preparations in native state.

Exploratory Scale Production of Tagless FabCys

Eukaryotic HEK293 cells were transfected with mammalian expression vector DNA encoding both heavy and light chains of disulfide-bridged FabCys. Cell culture supernatants were harvested on day 3 post transfection and subjected to CH1 affinity chromatography (Capture Select CH1-XL|Thermo Scientific). If not stated otherwise, buffer exchange was performed to 1×Dulbecco's PBS (pH 7.2|Invitrogen) and samples were sterile filtered (0.2 μm pore size).

Protein concentrations were determined by UV-spectrophotometry and purity of FabCys was analyzed under denaturing, reducing and non-reducing conditions using CE-SDS (LabChip GXII|Perkin Elmer|USA). HP-SEC was performed to analyze FabCys preparations in native state.

Production Results:

A summary of the quality control of the mammalian produced FabCys and human IgG1s are summarized in Tables 19-21.

This data reveal that the antibodies could be produced in acceptable yields, monomer content and purity. For 3 IgGs, the monomer content was determined in the range of 85% to 90%. All produced IgGs were subjected to preparatory size exclusion chromatography (Prep-SEC). Prep-SEC preparations resulted in monomer content of greater 93% for all produced IgGs as shown in Table 21.

TABLE 19
Production results of selected CD3 specific antibodies of the present
disclosure produced in the monovalent human FabCys format.

| | CE-SDS | | UHP-SEC |
|---|---|---|---|
| | Purity red. | Purity non-red. | Monomer |
| Antibody | [%] | [%] | [%] |
| MAB-7_h_FabCys | >95 | >80 | 99.3 |
| MAB-8_h_FabCys | >95 | >80 | 97.7 |
| MAB-9_h_FabCys | >95 | >80 | 97.8 |
| MAB-12_h_FabCys | >95 | >80 | 98.2 |
| MAB-13_h_FabCys | >95 | >80 | 97.1 |
| MAB-16_h_FabCys | >95 | >80 | 98.3 |

TABLE 20
Production results of selected CD3 specific antibodies of the present
disclosure produced in the silent bivalent human IgG1_AEASS
format before preparatory size-exclusion chromatography

| | Volu- | CE-SDS | | UHP- |
|---|---|---|---|---|
| | metric | Purity | Purity | SEC |
| | Yield | red. | non-red. | Monomer |
| Antibody | [mg/L] | [%] | [%] | [%] |
| MAB-7_h_IgG1f_AEASS | 83.8 | >95 | >80 | 96.5 |
| MAB-8_h_IgG1f_AEASS | 57.4 | >95 | >80 | 93.5 |
| MAB-9_h_IgG1f_AEASS | 23.9 | >95 | >80 | 84.5 |
| MAB-12_h_IgG1f_AEASS | 46.0 | >95 | >80 | 89.9 |
| MAB-13_h_IgG1f_AEASS | 32.0 | >95 | >80 | 86.5 |
| MAB-16_h_IgG1f_AEASS | 50.0 | >95 | >80 | 90.4 |

TABLE 21
Production results of selected CD3 specific antibodies of
the present disclosure produced in the silent human IgG1-
AEASS format after preparatory size-exclusion chromatography

| | CE-SDS | | UHP-SEC |
|---|---|---|---|
| | Purity red. | Purity non-red. | Monomer |
| Antibody | [%] | [%] | [%] |
| MAB-7_h_IgG1f_AEASS | >95 | >80 | 99.5 |
| MAB-8_h_IgG1f_AEASS | >95 | >80 | 98.2 |
| MAB-9_h_IgG1f_AEASS | >95 | >80 | 93.3 |
| MAB-12_h_IgG1f_AEASS | >95 | >80 | 95.3 |
| MAB-13_h_IgG1f_AEASS | >95 | >80 | 94.3 |
| MAB-16_h_IgG1f_AEASS | >95 | >80 | 95.2 |

### Example 13: K_{D} Determination on Human and Cynomolgus CD3 Epsilon by Biolayer Interferometry (BLI)

For K_{D} determinations, monomer fractions of antibody protein (Fab-Cys) were used (at least 90% monomer content, as analyzed by analytical SEC). Affinity determination by determining kinetic rate constants was performed on an Octet HTX (FortéBIO, Sartorius AG) instrument as described below.

The different Fab-Cys samples diluted in assay buffer (D-PBS, 0.05% (v/v) PS 20, 0.1% (w/v) BSA) were captured onto Fab-specific BLI sensors with a loading level of approx. 0.5 nm. For analysis, human CD3 epsilon antigen hCD3e(1-118)_F-chLys_avi (SEQ ID NO: 7) and cynomolgus monkey CD3 epsilon antigen cyCD3e(1-109)_F-chLys_avi (SEQ ID NO: 104) were diluted with assay buffer to concentrations ranging from 1.56 nM to 100 nM. A blank sample with assay buffer was included for referencing, i.e. correcting for dissociation of captured antibody. The association phase was recorded for 300 s, followed by a dissociation phase of 900 s. After each cycle, the biosensors were regenerated two times with 10 mM Glycine HCl pH 1.7 to remove bound ligand/antibody complex, while maintaining the integrity of the capture surface. Between regeneration steps, biosensors were washed with assay buffer for 20 s.

The sensorgrams were fitted using Octet Data Analysis Software 10.0 (FortéBio, Sartorius AG) to determine kₒₙ and k_{off} rate constants (using a 1:1 binding model), which were used to calculate K_{D}.

The results of the K_{D} determination are summarized in Table 22. The newly identified CD3 specific antibodies exhibited K_{D} values in a comparable range for the binding to recombinant human and cynomolgus monkey CD3 epsilon antigens.

TABLE 22
KD values of mammalian produced FabCys determined
on human and cynomolgus CD3 epsilon antigens.

| | hCD3e(1-118)_F-chLys_avi | | | cyCD3e(1-109)_F-chLys_avi | | |
|---|---|---|---|---|---|---|
| Fab | kₒₙ [1/Ms] | k_{off} [1/s] | K_{D} [nM] | kₒₙ [1/Ms] | k_{off} [1/s] | K_{D} [nM] |
| MAB-7 | 4.99E+05 | 6.27E−04 | 1.3 | 3.70E+05 | 1.58E−04 | 0.4 |
| MAB-8 | 8.02E+05 | 2.58E−04 | 0.3 | 4.64E+05 | 5.07E−05* | 0.1 |
| MAB-9 | 7.24E+05 | 1.22E−04 | 0.2 | 4.46E+05 | 5.00E−05* | 0.1 |
| MAB-12 | 6.50E+05 | 1.88E−04 | 0.3 | 4.45E+05 | 5.00E−05* | 0.1 |
| MAB-13 | 7.05E+05 | 1.34E−04 | 0.2 | 4.13E+05 | 5.00E−05* | 0.1 |
| MAB-16 | 8.52E+05 | 2.72E−04 | 0.3 | 4.78E+05 | 7.92E−05 | 0.2 |

*Dissociation approaching assay limit of the capture setup. Rate constants below 5E−05 1/s were set to this value (i.e. for these samples, the dissociation rate is maximally 5E−05 1/s). Therefore, K_{D} values were formatted with grey italic and should be considered as less precise, but may still serve as good estimation.

### Example 14: Cell Binding of CD3 Specific Antibodies to Human and Cynomolgus Derived PBMCs

Isolation of Human and Cynomolgus PBMCs

Human whole blood from healthy donors (in house) and whole blood from cynomolgus monkey (retrieved from LPT Laboratory of Pharmacology and Toxicology, Hamburg, Germany) was collected in Li-Heparin containing S-Monovette containers (Sarstedt). Blood was transferred to 50 ml conical tubes and mixed with an equal volume of PBS containing 2% fetal bovine serum (Sigma, #F7524) and 2 mM EDTA. Diluted blood was transferred to SepMate-50 tubes (StemCell Technologies, #86450) containing 15 ml Biocoll solution (Biochrom, #L6115) and centrifuged for 10 min at 1200×g. Supernatant was transferred into a 50 ml conical tube, diluted to 45 ml with PBS and centrifuged for 8 min at 300×g. Supernatant was discarded, cell pellet resuspended in 1 ml PBS and cells counted using a Neubauer chamber.

Cell Binding Assays

CD3 specific FabCys molecules according the present disclosure and an in-house prepared Fab-Cys of the prior art anti-CD3 antibody UCHT-1 (known to be not cross-reactive to cynomolgus monkey CD3) were tested by FACS for their ability to bind to human and cynomolgus derived PBMCs.

200,000 purified human or cynomolgus PBMCs (pre-blocked with in house produced recombinant human Fc2-H and goat anti-human IgG (Jackson Immuno Research, #109-005-097) or recombinant cynomolgus monkey IgG1 (R&D Systems, #9315-HG) and mouse anti-monkey IgG (Southern Biotech, SB108a), respectively) were mixed with antibodies serially diluted (final concentration: 0.012 nM-200 nM) in D-PBS (Gibco) containing 3% fetal bovine serum (Sigma, #F7524) and incubated for 1 h on ice. Bound antibodies were detected using goat anti human IgG-AF647 (Jackson Immuno Research, #109-606-097).

Antibody staining was measured using a multiformat Flow Cytometer (NovoCyte, Agilent Technologies) and analyzed using NovoExpress software (Version 1.5.0). Lymphocytes were identified by morphological gating of forward and side scatters. EC₅₀ values were calculated using 4-parameter non-linear regression analysis in Prism software (GraphPad Software Inc., version 8.4.3).

The results of the experiments are shown in Table 23 and reveal that the newly identified CD3 specific antibodies exhibited comparable binding to human and cynomolgus PBMCs. As expected, control Fab of UCHT-1 showed binding to human but no binding to cynomolgus monkey PBMCs at a concentration of 200 nM.

TABLE 23
Cell binding of CD3 specific FabCys antibodies to
human and cynomolgus monkey PBMCs expressing CD3. Shown
are EC₅₀ values. Control FabCys UCHT-1 was tested
at a single concentration of 200 nM.

EC₅₀ [nM]

| | human PBMCs | cyno PBMCs |
|---|---|---|
| MAB-7_h_FabCys | 6.1 | 3.7 |
| MAB-8_h_FabCys | 5.5 | 3.2 |
| MAB-9_h_FabCys | 7.7 | 4.7 |
| MAB-12_h_FabCys | 5.7 | 4.1 |
| MAB-13_h_FabCys | 8.3 | 5.2 |
| MAB-16_h_FabCys | 3.3 | 2.0 |
| UCHT-1_FabCys | binding | no binding |

1: An isolated human antibody or antibody fragment specific for human CD3 epsilon, wherein said antibody or antibody fragment comprises:
a) an HCDR1 region comprising the amino acid sequence of GFTFX₁SX₂X₃MX₄, wherein X₁ is S, K or R; X₂ is Y or H; X₃ is W or Y; and X₄ is S or T (SEQ ID NO: 90);
b) an HCDR2 region comprising the amino acid sequence of NIX₁X₂X₃X₄X₅X₆X₇YYX_{B}X₉SVKG, wherein X₁ is K or D; X₂ is Q or Y; X₃ is D, Q or E; X₄ is S or G; X₅ is S, Q or T; X₆ is E, H or R; X₇ is K, A or T; X₈ is V or A; and X₉ is D or E (SEQ ID NO: 91);
c) an HCDR3 region comprising the amino acid sequence of GYSAEFAHRSGLDV (SEQ ID NO: 39);
d) a LCDR1 region comprising the amino acid sequence of SGSSSNIGX₁X₂YVY, wherein X₁ is S, I, A, K or Q; and X₂ is N or T (SEQ ID NO: 92);
e) a LCDR2 region comprising the amino acid sequence of RNX₁X₂RPS, wherein X₁ is N, K, S, H, T or Y; and X₂ is Q, I or K (SEQ ID NO: 93); and
f) a LCDR3 region comprising the amino acid sequence of AX₁WDX₂X₃X₄X₅GAV, wherein X₁ is A or G; X₂ is H or R; X₃ is H or R; X₄ is R, S or L; and X₅ is S or H (SEQ ID NO: 94).

2: The isolated human antibody or antibody fragment specific for human CD3 epsilon according to claim 1, wherein said antibody or antibody fragment comprises:
a) an HCDR1 region comprising the amino acid sequence of GFTFSSYWMS (SEQ ID NO: 40), GFTFKSYYMS (SEQ ID NO: 41) or GFTFRSHYMT (SEQ ID NO: 42);
b) an HCDR2 region comprising the amino acid sequence of NIKQDGSEKYYVDSVKG (SEQ ID NO: 43), NIDYQSQHAYYAESVKG (SEQ ID NO: 44) or NIDYEGTRTYYAESVKG (SEQ ID NO: 45);
c) an HCDR3 region comprising the amino acid sequence of GYSAEFAHRSGLDV (SEQ ID NO: 39);
d) a LCDR1 region comprising the amino acid sequence of SGSSSNIGSNYVY (SEQ ID NO: 46), SGSSSNIGINYVY (SEQ ID NO: 47), SGSSSNIGANYVY (SEQ ID NO: 48), SGSSSNIGQTYVY (SEQ ID NO: 49), or SGSSSNIGKNYVY (SEQ ID NO: 50);
e) a LCDR2 region comprising the amino acid sequence of RNNQRPS (SEQ ID NO: 51), RNNIRPS (SEQ ID NO: 52), RNNKRPS (SEQ ID NO: 53), RNKKRPS (SEQ ID NO: 54), RNKQRPS (SEQ ID NO: 55), RNSQRPS (SEQ ID NO: 56), RNHIRPS (SEQ ID NO: 57), RNTQRPS (SEQ ID NO: 58), or RNYQRPS (SEQ ID NO: 59); and
f) a LCDR3 region comprising the amino acid sequence of AAWDHHRSGAV (SEQ ID NO: 60), AAWDRHSHGAV (SEQ ID NO: 61) or AGWDRRLHGAV (SEQ ID NO: 62).

3: The isolated human antibody or antibody fragment specific for human CD3 epsilon according to claim 1, wherein said antibody or antibody fragment comprises a variable heavy chain (VH) comprising the amino acid sequence of SEQ ID NO: 13, SEQ ID NO: 14, or SEQ ID NO: 15.

4: The isolated human antibody or antibody fragment specific for human CD3 epsilon according to claim 1, wherein said antibody or antibody fragment comprises a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

5: The isolated human antibody or antibody fragment specific for human CD3 epsilon according to claim 1, wherein said antibody or antibody fragment comprises
a) a variable heavy chain (VH) comprising the amino acid sequence of SEQ ID NO: 13, SEQ ID NO: 14, or SEQ ID NO: 15; and
b) a variable light chain (VL) comprising an amino acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

6: The isolated human antibody or antibody fragment specific for human CD3 epsilon according to claim 1, wherein the variable heavy chain and variable light chain are selected from the group consisting of:
a) the VH comprising the amino acid sequence of SEQ ID NO: 13 and the VL comprising the amino acid sequence of SEQ ID NO: 16,
b) the VH comprising the amino acid sequence of SEQ ID NO: 14 and the VL comprising the amino acid sequence of SEQ ID NO: 16,
c) the VH comprising the amino acid sequence of SEQ ID NO: 13 and the VL comprising the amino acid sequence of SEQ ID NO: 17,
d) the VH comprising the amino acid sequence of SEQ ID NO: 14 and the VL comprising the amino acid sequence of SEQ ID NO: 17,
e) the VH comprising the amino acid sequence of SEQ ID NO: 14 and the VL comprising the amino acid sequence of SEQ ID NO: 18,
f) the VH comprising the amino acid sequence of SEQ ID NO: 14 and the VL comprising the amino acid sequence of SEQ ID NO: 19,
g) the VH comprising the amino acid sequence of SEQ ID NO: 14 and the VL comprising the amino acid sequence of SEQ ID NO: 20,
h) the VH comprising the amino acid sequence of SEQ ID NO: 14 and the VL comprising the amino acid sequence of SEQ ID NO: 21,
i) the VH comprising the amino acid sequence of SEQ ID NO: 14 and the VL comprising the amino acid sequence of SEQ ID NO: 22,
j) the VH comprising the amino acid sequence of SEQ ID NO: 14 and the VL comprising the amino acid sequence of SEQ ID NO: 23,
k) the VH comprising the amino acid sequence of SEQ ID NO: 14 and the VL comprising the amino acid sequence of SEQ ID NO: 24,
l) the VH comprising the amino acid sequence of SEQ ID NO: 14 and the VL comprising the amino acid sequence of SEQ ID NO: 25,
m) the VH comprising the amino acid sequence of SEQ ID NO: 14 and the VL comprising the amino acid sequence of SEQ ID NO: 26,
n) the VH comprising the amino acid sequence of SEQ ID NO: 14 and the VL comprising the amino acid sequence of SEQ ID NO: 27,
o) the VH comprising the amino acid sequence of SEQ ID NO: 14 and the VL comprising the amino acid sequence of SEQ ID NO: 28,
p) the VH comprising the amino acid sequence of SEQ ID NO: 14 and the VL comprising the amino acid sequence of SEQ ID NO: 29,
q) the VH comprising the amino acid sequence of SEQ ID NO: 14 and the VL comprising the amino acid sequence of SEQ ID NO: 30,
r) the VH comprising the amino acid sequence of SEQ ID NO: 14 and the VL comprising the amino acid sequence of SEQ ID NO: 31,
s) the VH comprising the amino acid sequence of SEQ ID NO: 14 and the VL comprising the amino acid sequence of SEQ ID NO: 32,
t) the VH comprising the amino acid sequence of SEQ ID NO: 14 and the VL comprising the amino acid sequence of SEQ ID NO: 33,
u) the VH comprising the amino acid sequence of SEQ ID NO: 14 and the VL comprising the amino acid sequence of SEQ ID NO: 34,
v) the VH comprising the amino acid sequence of SEQ ID NO: 14 and the VL comprising the amino acid sequence of SEQ ID NO: 35,
w) the VH comprising the amino acid sequence of SEQ ID NO: 14 and the VL comprising the amino acid sequence of SEQ ID NO: 36,
x) the VH comprising the amino acid sequence of SEQ ID NO: 14 and the VL comprising the amino acid sequence of SEQ ID NO: 37,
y) the VH comprising the amino acid sequence of SEQ ID NO: 14 and the VL comprising the amino acid sequence of SEQ ID NO: 38,
z) the VH comprising the amino acid sequence of SEQ 1D NO: 15 and the VL comprising the amino acid sequence of SEQ 1D NO: 17,
aa) the VH comprising the amino acid sequence of SEQ ID NO: 15 and the VL comprising the amino acid sequence of SEQ ID NO: 18,
bb) the VH comprising the amino acid sequence of SEQ ID NO: 15 and the VL comprising the amino acid sequence of SEQ ID NO: 19,
cc) the VH comprising the amino acid sequence of SEQ ID NO: 15 and the VL comprising the amino acid sequence of SEQ ID NO: 20,
dd) the VH comprising the amino acid sequence of SEQ ID NO: 15 and the VL comprising the amino acid sequence of SEQ ID NO: 21,
ee) the VH comprising the amino acid sequence of SEQ ID NO: 15 and the VL comprising the amino acid sequence of SEQ ID NO: 22,
ff) the VH comprising the amino acid sequence of SEQ ID NO: 15 and the VL comprising the amino acid sequence of SEQ ID NO: 23,
gg) the VH comprising the amino acid sequence of SEQ ID NO: 15 and the VL comprising the amino acid sequence of SEQ ID NO: 24,
hh) the VH comprising the amino acid sequence of SEQ ID NO: 15 and the VL comprising the amino acid sequence of SEQ ID NO: 25,
ii) the VH comprising the amino acid sequence of SEQ ID NO: 15 and the VL comprising the amino acid sequence of SEQ ID NO: 26,
jj) the VH comprising the amino acid sequence of SEQ ID NO: 15 and the VL comprising the amino acid sequence of SEQ ID NO: 27,
kk) the VH comprising the amino acid sequence of SEQ ID NO: 15 and the VL comprising the amino acid sequence of SEQ ID NO: 28,
ll) the VH comprising the amino acid sequence of SEQ ID NO: 15 and the VL comprising the amino acid sequence of SEQ ID NO: 29,
mm) the VH comprising the amino acid sequence of SEQ ID NO: 15 and the VL comprising the amino acid sequence of SEQ ID NO: 30,
nn) the VH comprising the amino acid sequence of SEQ ID NO: 15 and the VL comprising the amino acid sequence of SEQ ID NO: 31,
oo) the VH comprising the amino acid sequence of SEQ ID NO: 15 and the VL comprising the amino acid sequence of SEQ ID NO: 32,
pp) the VH comprising the amino acid sequence of SEQ ID NO: 15 and the VL comprising the amino acid sequence of SEQ ID NO: 33,
qq) the VH comprising the amino acid sequence of SEQ ID NO: 15 and the VL comprising the amino acid sequence of SEQ 1D NO: 34,
rr) the VH comprising the amino acid sequence of SEQ ID NO: 15 and the VL comprising the amino acid sequence of SEQ ID NO: 35,
ss) the VH comprising the amino acid sequence of SEQ ID NO: 15 and the VL comprising the amino acid sequence of SEQ ID NO: 36,
tt) the VH comprising the amino acid sequence of SEQ ID NO: 15 and the VL comprising the amino acid sequence of SEQ ID NO: 37, and
uu) the VH comprising the amino acid sequence of SEQ ID NO: 15 and the VL comprising the amino acid sequence of SEQ ID NO: 38.

7: The isolated human antibody or antibody fragment specific for human CD3 epsilon according to claim 1, wherein said antibody or antibody fragment cross-reactively binds to cynomolgus CD3 epsilon.

8: The isolated human antibody or antibody fragment specific for human CD3 epsilon according to claim 1, wherein said antibody or antibody fragment is a recombinant antibody or antibody fragment.

9: The isolated human antibody or antibody fragment specific for human CD3 epsilon according to claim 1, wherein said antibody or antibody fragment is a monoclonal antibody or antibody fragment.

10: The isolated human antibody or antibody fragment specific for human CD3 epsilon according to claim 1, wherein the antibody fragment is selected from the group consisting of a Fab, Fv, and scFv.

11: The isolated human antibody or antibody fragment specific for human CD3 epsilon according to claim 1, wherein the antibody is an IgG.

12: A multispecific antibody comprising the isolated human antibody or antibody fragment specific for human CD3 epsilon according to claim 1.

13: The multispecific antibody according to claim 12, wherein the multispecific antibody also specifically binds to a cancer associated antigen.

14: A pharmaceutical composition comprising the isolated human antibody or antibody fragment specific for human CD3 epsilon according to claim 1 and a pharmaceutically acceptable carrier or excipient.

15. (canceled)

16: A pharmaceutical composition comprising the multispecific antibody according to claim 12 and a pharmaceutically acceptable carrier or excipient.

17: A method of treating or delaying the progression of a cell proliferative disease in a subject in need thereof, said method comprising administering the isolated human antibody or antibody fragment specific for human CD3 epsilon according to claim 1 to the subject.

18: A method of treating or delaying the progression of a cell proliferative disease in a subject in need thereof, said method comprising administering the multispecific antibody according to claim 12 to the subject.
